SEQUENCE LISTING (119 sequences)

SEQ ID NO: 1 - protein, 925 aa, synthetic construct
  REGION 1..925: note = ENPP1 Amino Acid Sequence - Wild Type
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 827 aa, synthetic construct
  REGION 1..827: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 3 - protein, 1063 aa, synthetic construct
  REGION 1..1063: note = ENPP1 Polypeptide
FTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 1060 aa, synthetic construct
  REGION 1..1060: note = ENPP1 Polypeptide
GLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 1057 aa, synthetic construct
  REGION 1..1057: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
  REGION 1..227: note = Human IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 906 aa, Mus musculus
MERDGDQAGHGPRHGSAGNGRELESPAAASLLAPMDLGEEPLEKAERARPAKDPNTYKVLSLVLSVCVLTTILGCIFGLKPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 8 - protein, 925 aa, Oryctolagus cuniculus
MERDGCAGGGSRGGEGGRAPREGPAGNSRDPGRSHAAEAPGNPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDQGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDNMVGMLMDGLKELNLHRCLNLILVSDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNIFSNMQALFVGYGPGFKHGIEVDTFENIEVYNLMCDLLNLTPAPNNGTHGSLNHLLKNPVYTPKHPKEVHPLIQCPFTRNPRDNLGCSCNPSILPIEDFQTQFNLTVAEEKNIKHETLPYGRPRVLQKKNTICLLSQHQFMSGYSQDILMPLWTSYTVDRNDSFSTEDFSNCLYQDFRISLSPVHKCSFYKNNTKVSYGFLSPPQLNKNSRGIYSEALLTTNIVPMYQSFQVIWRYFHDTLLRKYAEERNGVNVVSGPVFDFDYDGRYDSLEILRQKRRVIRNQEILIPTHFFIVLTSCKDASQTPLHCENLDTLAFILPHRTDNSESCLHGKHESSWVEELLMLHRARITDVEHITGLSFYQQRKEPVSDILKLKTHLPTFSQED

SEQ ID NO: 9 - protein, 924 aa, Bos taurus
MERDSCAGGGSRGGEGGRGPREGLAGNGRDPGPGRAAEASGEPQAAASLLAPMDLGEEPLERAARARPAKDPNTYKVLSLVLSVCVLTTILGCIFGLKPSCAKEIKSCKGRCFERTFGNCRCDAACVDLGNCCLDYQETCIEPERIWTCTKFRCGEKRLSRSLCSCSDDCKDKGDCCINHGSVCRGEKSWAEEECDSIDEPQCPAGFETPPTLLFSLDGFRAEYLHTWGGLLPVISKLKTCGTYTKNMRPVYPTKTFPNHYSIVTGLYPESHGIIDNNIYDPQMNANFALKNKEKFNPEWYKGEPIWLTAKYQGLKTGTFFWPGSDVKINGIFPDIYKIYNVSVPFEERILAILKWLQLPKDERPHFYTLYLEEPDSSGHSYGPVSSEVIRALQRVDNMVGMLMDGLKELNLHRCLNLILISDHGMEQGSCKKYVYLNKYLGDTKDYKVVYGPAARLRPSDVPDKYYSFDYEGIAKNLSCQEPNQHFKPYLKHFLPKRLHFAKNDRIERLTFYLDPQWQLALNPSERKYCGGGFHGSDNTFLNMQALFIGYGPGFKHSTEVDSFENIEVYNLMCDLLNLTPAPNNGTHGSLNHLLSNPVYTPKHPKEVRPLVQCPFTRAPRESLDCSCDPSILPIVDFQTQLNLTMAEEKTIKRGALPYGRPRVLQNSTVCLLYQHQFVSGYSRDILMPLWTSYTIGRNDSFSTEDFSNCLYQDLRIPLSPVHKCSFYKNNAKLSYGLLSPPQLHKGSSQVYSEALLTTNIVPMYQSFQVIWHYLHGTLLQRYAEERNGLNVVSGPVFDSDYDGRYDSLETLKQNSKIIRNLEVLIPTHFFLVLTSCKNTSQTPLQCENLDAMAFILPHKTDNSESCAHGKHESLWVEELLKLHTARITDVEHITGLSFYQERKEPISDILKLKTHLPTFNQED

SEQ ID NO: 10 - protein, 926 aa, Papio hamadryas
MERDGCAGGGSQGGGKGGRGPREGLAGNGRDPSHGQASEAPGDPQAAASLLAPMDLGEEPLEKAAGARPAKDPNTYKVLSLVLSVCVLTTILGCIFGLKPSCAKEVKSCKGRCFERTFGNCRCDVACVDLGNCCLDYQETCIEPERIWTCNKFRCGEKRLSRSLCACSDDCKERGDCCINYSAVCQGEKSWVEETCENINEPQCPEGFEMPPTLLFSLDGFRAEYLHTWGGLLPVISKLKKCGTYAKNMRPVYPTKTFPNHYSIVTGLYPESHGIIDNKMYDPKMNASFSLKSKEKFNPEWYKGEPIWLTAKYQGLRSGTFFWPGSDVKINGIFPDIYKIYNGSVPFEERILAILKWLRLPKDERPHFYTLYLEEPDSSGHSYGPVSSEVIKALQRVDNMVGMLMDGLKELNLHQCLNLILISDHGMEQGSCKKYIYLNKYLGDTKNIKVIYGPAARLRPSDVPEKYYSFNYENIARNLSCREPNQHFKPYLKHFLPKRLHFAKSDRIEPLTFYLDPQWQLALSPSERKYCGSGFHGSDNVFSNMQALFVGYGPGFQHGIEVDSFENIEVYNLMCDLLNLTPAPNNGTHGSLNHLLKNPIYTPKHPKEVQPSVQCPLAGSPRDSLGCSCNPSILPIVDFQTQFNLTTAEEKNINRASLPYGRPRLLQKKSSVCLLYQHQFVSGYSHDVLMPLWTSYTVNRNDSFSTEDFSNCLYQDLRISFSPIHNCSFYKNNAKLSYGFLSPPQLSKDSSQIYSEALLTSNIVPMYQSFQVIWRYFHDTLLQRYAEERNSINVVSGPVFDSDYDGRYDSSEALKRNRRVIRNQEILIPTHFFIVITSCKNTSQTPLQCDNLDPLAFILPHRSDNSESCVHEKRESSWIEELLMMHRARIMDVEHITGLSFYQERKEPVSDILKLKTHLPTVSQED

SEQ ID NO: 11 - protein, 15 aa, synthetic construct
  REGION 1..15: note = ENPP1 Polypeptide
  REGION 2..15: note = MISC_FEATURE - Features section of the listing that any one or all of aminoacids 2-15 can either be present or absent
DDDDDDDDDDDDDDD

SEQ ID NO: 12 - protein, 30 aa, synthetic construct
  REGION 1..30: note = ENPP1 Polypeptide
  REGION 4..30: note = MISC_FEATURE - Amino acids 4-30 can either be present or absent in groups of3(DSS) starting at position 4
DSSDSSDSSDSSDSSDSSDSSDSSDSSDSS

SEQ ID NO: 13 - protein, 30 aa, synthetic construct
  REGION 1..30: note = ENPP1 Polypeptide
  REGION 4..30: note = misc_feature - Amino acids 4-30 can either be present or absent in groups of3(ESS) starting at position 4
ESSESSESSESSESSESSESSESSESSESS

SEQ ID NO: 14 - protein, 30 aa, synthetic construct
  REGION 1..30: note = ENPP1 Polypeptide
  REGION 4..30: note = MISC_FEATURE - Amino acids 4-30 can either be present or absent in groups of3(RQQ) starting at position 4
RQQRQQRQQRQQRQQRQQRQQRQQRQQRQQ

SEQ ID NO: 15 - protein, 20 aa, synthetic construct
  REGION 1..20: note = ENPP1 Polypeptide
  REGION 3..20: note = MISC_FEATURE - Amino acids 3-20 can either be present or absent in groups of2(KR) starting at position 3
KRKRKRKRKRKRKRKRKRKR

SEQ ID NO: 16 - protein, 15 aa, synthetic construct
  REGION 1..15: note = ENPP1 Polypeptide
  REGION 2..15: note = MISC_FEATURE - Features section of the listing that any one or all of aminoacids 2-15 can either be present or absent
RRRRRRRRRRRRRRR

SEQ ID NO: 17 - protein, 16 aa, synthetic construct
  REGION 1..16: note = ENPP1 Polypeptide
DSSSEEKFLRRIGRFG

SEQ ID NO: 18 - protein, 12 aa, synthetic construct
  REGION 1..12: note = ENPP1 Polypeptide
EEEEEEEPRGDT

SEQ ID NO: 19 - protein, 12 aa, synthetic construct
  REGION 1..12: note = ENPP1 Polypeptide
APWHLSSQYSRT

SEQ ID NO: 20 - protein, 12 aa, synthetic construct
  REGION 1..12: note = ENPP1 Polypeptide
STLPIPHEFSRE

SEQ ID NO: 21-26 - skipped (6 entries)

SEQ ID NO: 27 - protein, 4 aa, synthetic construct
  REGION 1..4: note = Linker
GGGA

SEQ ID NO: 28 - protein, 4 aa, synthetic construct
  REGION 1..4: note = Linker
GGGS

SEQ ID NO: 29 - protein, 4 aa, synthetic construct
  REGION 1..4: note = Linker
GGGG

SEQ ID NO: 30 - protein, 5 aa, synthetic construct
  REGION 1..5: note = Linker
GGGGA

SEQ ID NO: 31 - protein, 5 aa, synthetic construct
  REGION 1..5: note = Linker
GGGGS

SEQ ID NO: 32 - protein, 5 aa, synthetic construct
  REGION 1..5: note = Linker
GGGGG

SEQ ID NO: 33 - protein, 4 aa, synthetic construct
  REGION 1..4: note = Linker
GGAG

SEQ ID NO: 34 - protein, 4 aa, synthetic construct
  REGION 1..4: note = Linker
GGSG

SEQ ID NO: 35 - protein, 4 aa, synthetic construct
  REGION 1..4: note = Linker
AGGG

SEQ ID NO: 36 - protein, 5 aa, synthetic construct
  REGION 1..5: note = Linker
SGGGG

SEQ ID NO: 37 - protein, 4 aa, synthetic construct
  REGION 1..4: note = Linker
SGGG

SEQ ID NO: 38 - protein, 4 aa, synthetic construct
  REGION 1..4: note = Linker
GAGA

SEQ ID NO: 39 - protein, 4 aa, synthetic construct
  REGION 1..4: note = Linker
GSGS

SEQ ID NO: 40 - protein, 6 aa, synthetic construct
  REGION 1..6: note = Linker
GAGAGA

SEQ ID NO: 41 - protein, 6 aa, synthetic construct
  REGION 1..6: note = Linker
GSGSGS

SEQ ID NO: 42 - protein, 8 aa, synthetic construct
  REGION 1..8: note = Linker
GAGAGAGA

SEQ ID NO: 43 - protein, 8 aa, synthetic construct
  REGION 1..8: note = Linker
GSGSGSGS

SEQ ID NO: 44 - protein, 10 aa, synthetic construct
  REGION 1..10: note = Linker
GAGAGAGAGA

SEQ ID NO: 45 - protein, 10 aa, synthetic construct
  REGION 1..10: note = Linker
GSGSGSGSGS

SEQ ID NO: 46 - protein, 12 aa, synthetic construct
  REGION 1..12: note = Linker
GAGAGAGAGAGA

SEQ ID NO: 47 - protein, 12 aa, synthetic construct
  REGION 1..12: note = Linker
GSGSGSGSGSGS

SEQ ID NO: 48 - protein, 6 aa, synthetic construct
  REGION 1..6: note = Linker
GGAGGA

SEQ ID NO: 49 - protein, 6 aa, synthetic construct
  REGION 1..6: note = Linker
GGSGGS

SEQ ID NO: 50 - protein, 9 aa, synthetic construct
  REGION 1..9: note = Linker
GGAGGAGGA

SEQ ID NO: 51 - protein, 9 aa, synthetic construct
  REGION 1..9: note = Linker
GGSGGSGGS

SEQ ID NO: 52 - protein, 12 aa, synthetic construct
  REGION 1..12: note = Linker
GGAGGAGGAGGA

SEQ ID NO: 53 - protein, 12 aa, synthetic construct
  REGION 1..12: note = Linker
GGSGGSGGSGGS

SEQ ID NO: 54 - protein, 4 aa, synthetic construct
  REGION 1..4: note = Linker
GGAG

SEQ ID NO: 55 - protein, 4 aa, synthetic construct
  REGION 1..4: note = Linker
GGSG

SEQ ID NO: 56 - protein, 4 aa, synthetic construct
  REGION 1..4: note = Linker
GGAG

SEQ ID NO: 57 - protein, 4 aa, synthetic construct
  REGION 1..4: note = Linker
GGSG

SEQ ID NO: 58 - protein, 8 aa, synthetic construct
  REGION 1..8: note = Linker
GGAGGGAG

SEQ ID NO: 59 - protein, 8 aa, synthetic construct
  REGION 1..8: note = Linker
GGSGGGSG

SEQ ID NO: 60 - protein, 12 aa, synthetic construct
  REGION 1..12: note = Linker
GGAGGGAGGGAG

SEQ ID NO: 61 - protein, 12 aa, synthetic construct
  REGION 1..12: note = Linker
GGSGGGSGGGSG

SEQ ID NO: 62 - protein, 5 aa, synthetic construct
  REGION 1..5: note = Linker
GGGGA

SEQ ID NO: 63 - protein, 5 aa, synthetic construct
  REGION 1..5: note = Linker
GGGGS

SEQ ID NO: 64 - protein, 15 aa, synthetic construct
  REGION 1..15: note = Linker
GGGGAGGGGAGGGGA

SEQ ID NO: 65 - protein, 15 aa, synthetic construct
  REGION 1..15: note = Linker
GGGGSGGGGSGGGGS

SEQ ID NO: 66 - protein, 5 aa, synthetic construct
  REGION 1..5: note = Linker
GGGAG

SEQ ID NO: 67 - protein, 6 aa, synthetic construct
  REGION 1..6: note = Linker
GGGAGG

SEQ ID NO: 68 - protein, 7 aa, synthetic construct
  REGION 1..7: note = Linker
GGGAGGG

SEQ ID NO: 69 - skipped

SEQ ID NO: 70 - protein, 5 aa, synthetic construct
  REGION 1..5: note = Linker
TGGGG

SEQ ID NO: 71 - protein, 4 aa, synthetic construct
  REGION 1..4: note = Linker
AAAL

SEQ ID NO: 72 - protein, 4 aa, synthetic construct
  REGION 1..4: note = Linker
AAAK

SEQ ID NO: 73 - protein, 4 aa, synthetic construct
  REGION 1..4: note = Linker
AAAR

SEQ ID NO: 74 - protein, 14 aa, synthetic construct
  REGION 1..14: note = Linker
EGKSSGSGSESKST

SEQ ID NO: 75 - protein, 12 aa, synthetic construct
  REGION 1..12: note = Linker
GSAGSAAGSGEF

SEQ ID NO: 76 - protein, 12 aa, synthetic construct
  REGION 1..12: note = Linker
AEAAAKEAAAKA

SEQ ID NO: 77 - protein, 18 aa, synthetic construct
  REGION 1..18: note = Linker
KESGSVSSEQLAQFRSLD

SEQ ID NO: 78 - protein, 10 aa, synthetic construct
  REGION 1..10: note = Linker
GENLYFQSGG

SEQ ID NO: 79 - protein, 8 aa, synthetic construct
  REGION 1..8: note = Linker
SACYCELS

SEQ ID NO: 80 - protein, 5 aa, synthetic construct
  REGION 1..5: note = Linker
RSIAT

SEQ ID NO: 81 - protein, 17 aa, synthetic construct
  REGION 1..17: note = Linker
RPACKIPNDLKQKVMNH

SEQ ID NO: 82 - protein, 36 aa, synthetic construct
  REGION 1..36: note = Linker
GGSAGGSGSGSSGGSSGASGTGTAGGTGSGSGTGSG

SEQ ID NO: 83 - protein, 17 aa, synthetic construct
  REGION 1..17: note = Linker
AAANSSIDLISVPVDSR

SEQ ID NO: 84 - protein, 36 aa, synthetic construct
  REGION 1..36: note = Linker
GGSGGGSEGGGSEGGGSEGGGSEGGGSEGGGSGGGS

SEQ ID NO: 85 - protein, 5 aa, synthetic construct
  REGION 1..5: note = Linker
EAAAK

SEQ ID NO: 86 - protein, 5 aa, synthetic construct
  REGION 1..5: note = Linker
PAPAP

SEQ ID NO: 87-90 - skipped (4 entries)

SEQ ID NO: 91 - protein, 15 aa, synthetic construct
  REGION 1..15: note = Linker
  REGION 1..15: note = MISC_FEATURE - Features section of the listing that any one or all of aminoacids 1-15 can either be present or absent
RRRRRRRRRRRRRRR

SEQ ID NO: 92 - protein, 16 aa, synthetic construct
  REGION 1..16: note = Linker
DSSSEEKFLRRIGRFG

SEQ ID NO: 93 - protein, 12 aa, synthetic construct
  REGION 1..12: note = Linker
EEEEEEEPRGDT

SEQ ID NO: 94 - protein, 12 aa, synthetic construct
  REGION 1..12: note = Linker
APWHLSSQYSRT

SEQ ID NO: 95 - protein, 12 aa, synthetic construct
  REGION 1..12: note = Linker
STLPIPHEFSRE

SEQ ID NO: 96 - protein, 12 aa, synthetic construct
  REGION 1..12: note = Linker
VTKHLNQISQSY

SEQ ID NO: 97 - protein, 15 aa, synthetic construct
  REGION 1..15: note = Linker
  REGION 2..15: note = MISC_FEATURE - Features section of the listing that any one or all of aminoacids 2-15 can either be present or absent
EEEEEEEEEEEEEEE

SEQ ID NO: 98 - protein, 7 aa, synthetic construct
  REGION 1..7: note = Linker
RSGSGGS

SEQ ID NO: 99 - protein, 15 aa, synthetic construct
  REGION 1..15: note = Linker
  REGION 2..15: note = MISC_FEATURE - Features section of the listing that any one or all of aminoacids 1-15 can either be present or absent
DDDDDDDDDDDDDDD

SEQ ID NO: 100 - protein, 16 aa, synthetic construct
  REGION 1..16: note = Linker
LVIMSLGLGLGLGLRK

SEQ ID NO: 101 - protein, 15 aa, synthetic construct
  REGION 1..15: note = Linker
VIMSLGLGLGLGLRK

SEQ ID NO: 102 - protein, 14 aa, synthetic construct
  REGION 1..14: note = Linker
IMSLGLGLGLGLRK

SEQ ID NO: 103 - protein, 13 aa, synthetic construct
  REGION 1..13: note = Liniker
MSLGLGLGLGLRK

SEQ ID NO: 104 - protein, 12 aa, synthetic construct
  REGION 1..12: note = Linker
SLGLGLGLGLRK

SEQ ID NO: 105 - protein, 11 aa, synthetic construct
  REGION 1..11: note = Linker
LGLGLGLGLRK

SEQ ID NO: 106 - protein, 10 aa, synthetic construct
  REGION 1..10: note = Linker
GLGLGLGLRK

SEQ ID NO: 107 - protein, 9 aa, synthetic construct
  REGION 1..9: note = Linker
LGLGLGLRK

SEQ ID NO: 108 - protein, 8 aa, synthetic construct
  REGION 1..8: note = Linker
GLGLGLRK

SEQ ID NO: 109 - protein, 7 aa, synthetic construct
  REGION 1..7: note = Linker
LGLGLRK

SEQ ID NO: 110 - protein, 6 aa, synthetic construct
  REGION 1..6: note = Linker
GLGLRK

SEQ ID NO: 111 - protein, 5 aa, synthetic construct
  REGION 1..5: note = Linker
LGLRK

SEQ ID NO: 112 - protein, 4 aa, synthetic construct
  REGION 1..4: note = Linker
GLRK

SEQ ID NO: 113-114 - skipped (2 entries)

SEQ ID NO: 115 - protein, 15 aa, synthetic construct
  REGION 1..15: note = Linker
  REGION 2..15: note = MISC_FEATURE - Features section of the listing that any one or all of aminoacids 2-15 can either be present or absent
KKKKKKKKKKKKKKK

SEQ ID NO: 116 - protein, 12 aa, synthetic construct
  REGION 1..12: note = ENPP1 Polypeptide
VTKHLNQISQSY

SEQ ID NO: 117 - protein, 14 aa, synthetic construct
  REGION 1..14: note = ENPP1 Polypeptide
  REGION 2..14: note = MISC_FEATURE - Features section of the listing that any one or all of aminoacids 2-15 can either be present or absent
EEEEEEEEEEEEEE

SEQ ID NO: 118 - protein, 1078 aa, synthetic construct
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGTFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 1086 aa, synthetic construct
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGTFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDD

(19) United States
(12) Patent Application Publication
(10) Pub. No.: US 2026/0041742 A1
(43) Pub. Date: Feb. 12, 2026

(54) METHOD AND COMPOSITIONS FOR TREATMENT, AMELIORATION, AND/OR PREVENTION OF DIFFUSE IDIOPATHIC SKELETAL HYPEROSTOSIS (DISH)

(71) Applicant: Yale University, New Haven, CT (US)

(72) Inventor: Demetrios Braddock, New Haven, CT (US)

(21) Appl. No.: 18/851,844

(22) PCT Filed: Sep. 30, 2022
(86) PCT No.: PCT/US2022/077407

§ 371 Date: Sep. 27, 2024

Related U.S. Application Data
(60) Provisional application No. 63/325,554, filed on Mar. 30, 2022.

(51) Int. Cl. A61K 38/46; A61K 47/64; A61K 47/66; A61K 47/68; A61P 19/02

(52) U.S. Cl. CPC A61K 38/465; A61K 47/643; A61K 47/66; A61K 47/6815; A61P 19/02; C12Y 301/04001; C12Y 306/01009

(57) ABSTRACT

The present disclosure provides, in one aspect, specific doses of an ENPP1 agent for in vivo treatment of Diffuse idiopathic skeletal hyperostosis (DISH), Ankylosing Spondylitis, and/or Spondylarthritis.

### CROSS-REFERENCE TO RELATED APPLICATIONS

This application is a 35 U.S.C. § 371 national phase application from, and claims priority to, PCT International Patent Application No. PCT/US2022/077407 filed Sep. 30, 2022, which claims priority to U.S. Provisional Patent Application No. 63/325,554 filed Mar. 30, 2022, the contents of all of which are incorporated hereby by reference in its their entireties herein.

### STATEMENT REGARDING FEDERALLY SPONSORED RESEARCH AND DEVELOPMENT

This invention was made with government support under DK121326-0 and AR080416-01 awarded by National Institutes of Health. The government has certain rights in the invention.

### FIELD

The field of the invention relates, in one aspect, to treatment and/or amelioration of DISH, Ankylosing Spondylitis, and/or Spondylarthritis by enzyme therapy.

### SEQUENCE LISTING

This XML filed named “047162-7377WO1(01806) Sequence Listing.xml”, created Sep. 29, 2022, comprising 149 Kbytes in size, is hereby incorporated by reference in its entirety.

### BACKGROUND

Ectonucleotide pyrophosphatase/phosphodiesterase 1 (ENPP1) is a type-2 transmembrane protein whose extracellular activity hydrolyzes phosphodiester bonds of extracellular nucleotides, such as adenosine triphosphate (ATP), to generate adenosine monophosphate (AMP) and inorganic pyrophosphate (PPi). Because PPi is the main physiologic inhibitor of hydroxyapatite deposition, biallelic ENPP1 deficiency leads to ectopic mineralization in early life, with high mortality in infancy resulting from arterial calcification and luminal narrowing (Ziegler et al., Generalized arterial calcification of infancy. In: Adam M P, Ardinger H H, Pagon R A, et al., (eds). Gene Reviews 2014). Patients who survive, as well as ENPP1-deficient individuals who do not exhibit neonatal calcifications, can develop fibroblast growth factor 23 (FGF23)-mediated hypophosphatemic rickets in later life (Ferreira et al., Prospective phenotyping of long-term survivors of generalized arterial calcification of infancy (GACI), Genet Med. 2020; 23:396-407).

WO2014126965 relates to treatment of pathological calcification and pathological ossification in diseases involving ENPP1 deficiency. Three proteins—ENPP1, tissue-nonspecific alkaline phosphatase (TNAP), and progressive ankylosis protein or ankyrin (ANK)—play a role in regulating the extracellular balance of inorganic pyrophosphate (PPi) and phosphate (Pi). Inorganic pyrophosphate (PPi) is generated by the cleavage of extracellular nucleotide triphosphates (NTPs) by ENPP1 or the transfer of PPi from the intracellular to extracellular space by Ank. TNAP degrades PPi to generate Pi.

“ENPP1 Deficiency” is characterized by a reduced level of ENPP1 enzymatic activity in serum and/or plasma of a subject. ENPP1 Deficiency is a rare genetic disorder caused by inactivating mutations in the ENPP1 gene that encodes the ENPP1 enzyme. As described elsewhere herein, ENPP1 is an integral transmembrane protein whose extracellular domains carry pyrophosphatase and phosphodiesterase activities. As such, ENPP1 converts extracellular ATP to inorganic pyrophosphate (PPi) and AMP.

Calcification in biological systems is a complex process by which calcium salts are maintained at higher concentrations in noncirculating matrices than in regional circulating humoral or other mobile fluids. The principal result of normal calcification is the accumulation of calcium and associated inorganic salts in crystalline patterns of similar arrangement and chemical composition in specialized intercellular matrices, all of which might vary among species. On the other hand, the net effect of pathological calcification is the accumulation of calcium and associated inorganic salts with a greater-than-normal range in chemical composition or diversity of pattern, not only in these specialized matrices, but also in other intercellular, extracellular, and cellular materials thereby leading to several disease states.

Diffuse Idiopathic Skeletal Hyperostosis (DISH) is a skeletal disorder characterized by unusual, new bone formation (Resnick et al., Diffuse idiopathic skeletal hyperostosis (DISH): Forestier's disease with extraspinal manifestations, Radiology. 1975; 115:513-524). The new bone forms most often where ligaments and tendons join bone (entheseal area), but there is also a generalized hardening of bones and bone overgrowth (hyperostosis) (Pillai & Littlejohn, Metabolic Factors in Diffuse Idiopathic Skeletal Hyperostosis—A Review of Clinical Data. The Open Rheumatology Journal. 2014; 8:116-128). The respective roles of ENPP1, TNAP, and ANK in relation to DISH are not understood. Further, it is also not known whether a deficiency in one or more of these enzymes is part of a DISH phenotype.

### BRIEF SUMMARY

The disclosure provides a method of treating, ameliorating, and/or preventing diffuse idiopathic skeletal hyperostosis (DISH), Ankylosing Spondylitis, and/or Spondylarthritis in a patient in need thereof. In certain embodiments, the method comprises administering to the patient a therapeutically effective amount of a compound of formula (I), or a salt or solvate thereof:

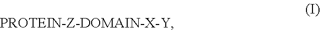

wherein in (I):
PROTEIN comprises the catalytic region of ENPP1;
DOMAIN is absent or at least one selected from the group consisting of a human IgG Fc domain (Fc), human serum albumin protein (ALB), and a fragment thereof;
X and Z are independently absent or a polypeptide comprising 1-20 amino acids,
Y is absent or a negatively charged bone-targeting sequence, thereby treating, ameliorating, and/or preventing DISH, Ankylosing Spondylitis, and/or Spondylarthritis in the patient.

In certain embodiments, the compound lacks a negatively charged bone-targeting sequence.

In certain embodiments, Y is absent.

In certain embodiments, the compound comprises a negatively charged bone-targeting sequence.

In certain embodiments, the patient has ENPP1 haploinsufficiency.

In certain embodiments, the patient does not have ENPP1 haploinsufficiency.

In certain embodiments, the patient is not ENPP1 deficient.

In certain embodiments, the patient is ENPP1 deficient.

In certain embodiments, the patient is administered the compound by at least one route selected from the group consisting of oral, aerosol, inhalational, rectal, vaginal, transdermal, subcutaneous, intranasal, buccal, sublingual, parenteral, intrathecal, intragastrical, ophthalmic, pulmonary, and topical.

In certain embodiments, the compound is intravenously or subcutaneously administered to the patient.

In certain embodiments, administering the compound to the patient increases, or prevents further decrease of, the patient's extracellular pyrophosphate concentrations.

In certain embodiments, administering the compound to the patient decreases, or prevents further increase of, one or more of calcification of Achilles tendon, spinal calcification, hip joint calcification, and bilateral calcification in the patient.

In certain embodiments, the DOMAIN comprises Albumin.

In certain embodiments, the DOMAIN comprises an IgG Fc domain.

In certain embodiments, the PROTEIN lacks the ENPP1 transmembrane domain.

In certain embodiments, the compound is administered to the patient as a pharmaceutical composition further comprising at least one pharmaceutically acceptable carrier.

In certain embodiments, the patient is a mammal.

In certain embodiments, the mammal is a human.

In certain embodiments, the PROTEIN comprises amino acid residues 99 (PSCAKE . . . ) to 925 ( . . . QED) of SEQ ID NO: 1.

In certain embodiments, the PROTEIN comprises amino acid residues 1 to 833 of SEQ ID NO: 3.

In certain embodiments, the PROTEIN comprises the amino acid sequence depicted in SEQ ID NO: 2.

In certain embodiments, the PROTEIN comprises the amino acid sequence depicted in SEQ ID NO: 3 or 4 or 5.

In certain embodiments, the DOMAIN increases the circulating half-life of the compound relative to the circulating half-life of the compound lacking the DOMAIN.

In certain embodiments, the patient has also been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

In certain embodiments, the patient has not been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

### DESCRIPTION

In certain embodiments, the disease or disorder contemplated herein is diffuse idiopathic skeletal hyperostosis (DISH). In certain embodiments, the disease or disorder contemplated herein is Ankylosing Spondylitis. In certain embodiments, the disease or disorder contemplated herein is Spondylarthritis. In certain embodiments, the disease or disorder contemplated herein is diffuse idiopathic skeletal hyperostosis (DISH) and/or Ankylosing Spondylitis. In certain embodiments, the disease or disorder contemplated herein is DISH and/or Spondylarthritis. In certain embodiments, the disease or disorder contemplated herein is Ankylosing Spondylitis and/or Spondylarthritis.

The invention provides a method of treating, ameliorating, preventing further development and/or progression of, and/or preventing diffuse idiopathic skeletal hyperostosis (DISH), Ankylosing Spondylitis, and/or Spondylarthritis in a patient in need thereof. The invention further provides a method of treating and/or ameliorating DISH, Ankylosing Spondylitis, and/or Spondylarthritis in a patient in need thereof.

In one aspect, the disclosure relates to treating, ameliorating, preventing further development and/or progression of, and/or preventing DISH, Ankylosing Spondylitis, and/or Spondylarthritis by administering to a subject having DISH, Ankylosing Spondylitis, and/or Spondylarthritis a therapeutically effective amount of an ENPP1 agent.

In one aspect, the disclosure relates to treating, ameliorating, preventing further development and/or progression of, and/or preventing ENPP1 Deficiency in a subject by administering to the subject a therapeutically effective amount of an ENPP1 agent.

In one aspect, the disclosure relates to treating, ameliorating, preventing further development and/or progression of, and/or preventing one or more symptoms of ENPP1 haploinsufficiency in a subject by administering to the subject a therapeutically effective amount of an ENPP1 agent.

Treatment of DISH, Ankylosing Spondylitis, and/or Spondylarthritis comprises administering an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, thereby treating, reducing, and/or ameliorating one or more symptoms of DISH, Ankylosing Spondylitis, and/or Spondylarthritis disease, wherein the subject does not have ENPP1 Deficiency.

In one aspect, the disclosure relates to administering an ENPP1 agent to a subject having DISH, Ankylosing Spondylitis, and/or Spondylarthritis at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, thereby treating, reducing, and/or ameliorating one or more symptoms of DISH, Ankylosing Spondylitis, and/or Spondylarthritis disease, wherein the subject has ENPP1 Deficiency.

In one aspect, the disclosure relates to administering to a subject having DISH, Ankylosing Spondylitis, and/or Spondylarthritis an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, thereby treating, reducing, and/or ameliorating one or more symptoms of DISH, Ankylosing Spondylitis, and/or Spondylarthritis disease, wherein the subject does not have ENPP1 haploinsufficiency.

In one aspect, the disclosure relates to administering to a subject having DISH, Ankylosing Spondylitis, and/or Spondylarthritis an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, thereby treating, reducing, and/or ameliorating one or more symptoms of DISH, Ankylosing Spondylitis, and/or Spondylarthritis disease, wherein the subject has ENPP1 haploinsufficiency.

In one aspect, the disclosure relates to administering to a subject having DISH, Ankylosing Spondylitis, and/or Spondylarthritis an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, in order to restore a physiological level of ENPP1 protein and/or activity in the plasma and/or tissues of the subject. A physiological level of ENPP1 protein and/or activity in the plasma and/or tissues, as used herein, is an amount or concentration of ENPP1 agent sufficient to achieve and maintain a physiological level of PPi in human serum. In certain embodiments, the ENPP1 agent is ENPP1 and/or an ENPP1 construct comprising ENPP1 activity.

In one aspect, the disclosure relates to administering to a subject having an ENPP1 haploinsufficiency an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, in order to restore a physiological level of ENPP1 protein and/or activity in the plasma and/or tissues of the subject. A physiological level of ENPP1 protein and/or activity in the plasma and/or tissues, as used herein, is an amount or concentration of ENPP1 agent sufficient to achieve and maintain a physiological level of PPi in human serum. In certain embodiments, the ENPP1 agent is ENPP1 and/or an ENPP1 construct comprising ENPP1 activity.

In once aspect, the disclosure relates to a method for increasing circulating pyrophosphate (PPi) in a subject with DISH, Ankylosing Spondylitis, and/or Spondylarthritis, the method comprising administering to the subject an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, to thereby increase circulating PPi in the subject.

In one aspect, the disclosure relates to a method for increasing circulating pyrophosphate (PPi) in a subject with ENPP1 haploinsufficiency, the method comprising administering to the subject an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, to thereby increase circulating PPi in the subject.

In one aspect, the disclosure relates to a method for ameliorating one or more symptoms of ENPP1 haploinsufficiency in a subject, the method comprising administering to the subject an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, to thereby ameliorate one or more symptoms of ENPP1 Deficiency or one or more symptoms of ENPP1 haploinsufficiency in the subject.

In an aspect, the disclosure relates to a method for preventing and/or reversing progression of, or minimizing and/or reducing, pathological calcification and/or the ossified masses present in a subject with DISH, Ankylosing Spondylitis, and/or Spondylarthritis, the method comprising administering to the subject an ENPP1 agent at a dose of about 0.1 mg per kilogram, about 0.2 mg per kilogram of the subject, about 0.3 mg per kilogram of the subject, about 0.4 mg per kilogram of the subject, about 0.5 mg per kilogram of the subject, about 0.6 mg per kilogram of the subject, about 0.7 mg per kilogram of the subject, about 0.8 mg per kilogram of the subject, about 0.9 mg per kilogram of the subject, about 1.0 mg per kilogram of the subject, about 1.1 mg per kilogram of the subject, about 1.2 mg per kilogram of the subject, about 1.3 mg per kilogram of the subject, about 1.4 mg per kilogram of the subject, about 1.5 mg per kilogram of the subject, about 1.6 mg per kilogram of the subject, about 1.7 mg per kilogram of the subject, about 1.8 mg per kilogram of the subject, about 1.9 mg per kilogram of the subject, or about 2.0 mg per kilogram of the subject, to thereby prevent and/or reverse progression of, or minimize and/or reduce, pathological calcification and/or the ossified skeletal masses in the subject.

In some embodiments of any of the aforesaid methods, the method comprises administering to the patient a therapeutically effective amount of an ENPP1 agent.

In some embodiments, the ENPP1 agent is a compound of formula (I), or a salt or solvate thereof:

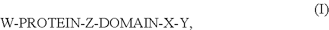

wherein in (I):
W is absent or comprises a signal sequence which allows for export of the compound into extracellular space;
PROTEIN comprises the catalytic region of ENPP1;
DOMAIN is absent or at least one selected from the group consisting of a human IgG Fc domain (Fc), human serum albumin protein (ALB), and a biologically active fragment thereof;
X and Z are independently absent or a polypeptide comprising 1-20 amino acids; and
Y is absent or a “bone targeting” sequence group
   wherein m is independently an integer ranging from 1 to 15, and
   wherein n is independently an integer ranging from 1 to 10.

In some embodiments, W is absent. In some embodiments, W comprises a signal sequence which allows for export of the compound into extracellular space.

In some embodiments, Y is a “bone targeting” sequence group selected from the group consisting of: Dₘ (SEQ ID NO:11), (DSS)ₙ (SEQ ID NO:12), (ESS)ₙ (SEQ ID NO:13), (RQQ)ₙ (SEQ ID NO:14), (KR)ₙ (SEQ ID NO:15), Rₘ (SEQ ID NO:16), DSSSEEKFLRRIGRFG (SEQ ID NO:17), EEEEEEEPRGDT (SEQ ID NO: 18), APWHLSSQYSRT (SEQ ID NO: 19), STLPIPHEFSRE (SEQ ID NO: 20), VTKHLNQISQSY (SEQ ID NO: 116), and Eₘ (SEQ ID NO: 117),

### BRIEF DESCRIPTION OF THE DRAWINGS

FIGS. 1A-1B show spine CT and bone scintigraphy of patient 1. FIG. 1A indicates the presence of multiple compression fractures in the spine detected by spine CT shown as white arrows. FIG. 1B shows bone scintigraphy that revealed multiple accumulations in the ribs.

FIGS. 1C-1H show X-rays of the hip joints, knee joints and Achilles tendon in patient 1. FIG. 1C: Right hip joint, FIG. 1D: Left hip joint, FIG. 1E: Right knee joint, FIG. 1F: Left knee joint, FIG. 1G: Right Achilles tendon, FIG. 1H: Left Achilles tendon. There was no sign of ectopic ossification.

FIGS. 2A-2C show the ENPP1 mutation family pedigree and correspond to patient 1, 2, and 3, respectively. The arrows indicate the probands.

FIGS. 3A-3G show the spine CT, X-rays of the hip joints, the knee joints and Achilles tendon in patient 2. FIG. 3A: Spine CT showed the paraspinal ligament ossification (white arrowheads) and multiple compression fractures (white arrows). FIGS. 3B-3C correspond to X-rays of the right hip joint (FIG. 3B) and left hip joint (FIG. 3C) respectively. Figures indicate that no sign of ectopic ossification was observed. FIGS. 3D-3E correspond to X-rays of the right knee joint (FIG. 3D) and left knee joint (FIG. 3E) respectively. Figures indicate that no sign of ectopic ossification was observed. FIG. 3F and FIG. 3G correspond to X-rays of the right Achilles tendon (FIG. 3F) and left Achilles tendon (FIG. 3G) respectively. Tibial sign of enthesopathy was detected in the right Achilles tendon (shown as white arrowhead).

FIGS. 4A-4I show the spine CT, X-rays of the hip joints, the knee joints and Achilles tendon in patient 3. FIGS. 4A-4C correspond to spine CT which showed multiple paraspinal ossifications in the cervical spine (FIG. 4A), thoracic spine (FIG. 4B) and lumbar spine (FIG. 4C) (white arrowheads). FIGS. 4D-4E show prominent ossifications around the right (FIG. 4D) and left (FIG. 4E) hip joints which were detected in X-rays (white arrowheads). FIGS. 4F-4G show X-rays of the right (FIG. 4F) and left (FIG. 4G) knee joints which present no sign of ectopic ossification. FIGS. 4H-4I indicate that prominent enthesopathy was detected in the x-rays of right (FIG. 4H) and left (FIG. 4I) Achilles tendon (white arrowheads).

FIGS. 5A-5G show spine CT and X-rays of the hip joints, knee joints and Achilles tendon in son of patient 1. (FIG. 5A) Spine CT, (FIG. 5B) Right hip joint, (FIG. 5C) Left hip joint, (FIG. 5D) Right knee joint, (FIG. 5E) Left knee joint, (FIG. 5F) Right Achilles tendon, (FIG. 5G) Left Achilles tendon. There was no sign of ectopic ossification.

FIGS. 6A-6G show spine CT and X-rays of the hip joints, knee joints and Achilles tendon in one son of patient 3. (FIG. 6A) Spine CT, (FIG. 6B) Right hip joint, (FIG. 6C) Left hip joint, (FIG. 6D) Right knee joint, (FIG. 6E) Left knee joint, (FIG. 6F) Right Achilles tendon, (FIG. 6G) Left Achilles tendon. There was slight enthespathy in the left Achilles tendon (white arrowhead).

FIGS. 7A-7G show spine CT and X-rays of the hip joints, knee joints and Achilles tendon in another son of case 3. (FIG. 7A) Spine CT, (FIG. 7B) Right hip joint, (FIG. 7C) Left hip joint, (FIG. 7D) Right knee joint, (FIG. 7E) Left knee joint, (FIG. 7F) Right Achilles tendon, (FIG. 7G) Left Achilles tendon. There was slight enthespathy in the left Achilles tendon (white arrowhead).

FIG. 8 shows Sanger sequencing of the complementary DNA confirmed compound heterozygosity for ENPP1 variants in patient 3. While clone type 1 contained ENPP1 variant c.536A>G, another ENPP1 variant c. 1352A>G was located in clone type 2, suggesting compound heterozygosity.

FIG. 9 shows a non-limiting schematic of ENPP1 structure and the location of the present variants. ENPP1 consists of cytoplasmic domain (CD), transmembrane domain (TM), and an extracellular domain composed of two somatomedin domains (SMB1 and SMB2), catalytic domain, and nuclease-like domain. N179S and Y451C located in SMB2 and catalytic domain, respectively.

FIG. 10 shows the comparison of the enzymatic activity of WT, N179S, and Y451C. When compared with WT ENPP1, the N179S and Y451C variants showed 55% and 70% reduced velocity of the enzymatic reaction, respectively. Bars indicate median and interquartile values. ****: p<0.0001.

FIG. 11 shows the full, unprocessed amino acid sequence of wild-type ENPP1 precursor protein (SEQ ID NO: 1). The cytosolic and transmembrane regions are underlined. Potential N-glycosylation sites are in bold. PSCAKE (residues 99-104; boxed) is the start of soluble ENPP1 protein portion which includes SMB1 (residues 104-144) and SMB2 (residues 145-189).

FIG. 12 shows response of plasma PPi (FIG. 12) in WT and Enpp1^{asj} mice dosed with weekly indicated subcutaneous doses of vehicle or ENPP1. *p<0.05, **p<0.01; (ANOVA, Kruskal-Wallis test).

FIG. 13 shows response of paraspinal osteophytes and ankylosis in 17-week old WT and 17-week old Enpp1^{asj} male mice dosed with weekly indicated subcutaneous doses of vehicle or ENPP1 constructs #1118 and #2000. Construct #1118 was dosed once a week beginning week 3, while Construct #2000 was dosed once a week beginning week 5. Micro-CT images demonstrate attenuation of paraspinal osteophytes and ankylosis preferentially in Construct #2000 and is apparent in both the Enpp1^{asj} male and female mice dosed at 1 mg/Kg weekly doses.

FIG. 14 shows response of paraspinal osteophytes and ankylosis in 17-week old WT and 17-week old Enpp1^{asj} female mice dosed with weekly indicated subcutaneous doses of vehicle or ENPP1 constructs #1118 and #2000. Construct #1118 was dosed once a week beginning week 3, while Construct #2000 was dosed once a week beginning week 5. Micro-CT images demonstrate attenuation of paraspinal osteophytes and ankylosis preferentially in Construct #2000 and is apparent in both the Enpp1^{asj} male and female mice dosed at 1 mg/Kg weekly doses.

FIGS. 15A-15B shows auditory brain stem response in 17-week old WT and 17-week old Enpp1^{asj} mice dosed with weekly indicated subcutaneous doses of vehicle or ENPP1 constructs #1118 and #2000. Construct #1118 was dosed once a week beginning week 3, while Construct #2000 was dosed once a week beginning week 5. Stimulus frequency measurements demonstrate prevention of hearing loss in the low frequency (8 kHz) range by weekly doses of 2 mg/Kg ENPP1 Construct #1118, and at weekly doses of 0.5 and 1 mg/Kg of ENPP1 Construct #2000. Improvement in hearing in ENPP1 deficient animals is also noted in high frequency range (32 kHz) preferentially in ENPP1 Construct #2000 dosed at weekly doses of 1 mg/Kg.

FIG. 16 illustrates intact FGF23 levels of WT and 17-week old ENPP1^{asj} mice with the weekly indicated subcutaneous doses of vehicle or ENPP1 Constructs #1118 and #2000. Construct #1118 was dosed once a week beginning week 3, while Construct #2000 was dosed once a week beginning week 5. The data demonstrates suppression of intact FGF23 preferentially in ENPP1 construct #2000 when dosed at 1 mg/Kg and 4 mg/Kg per week. Statistical significance was assessed by an ANOVA Kruskal-Wallis test followed by Dunn's post hoc analysis to evaluate differences with WT levels (one-way ANOVA). Statistical significance is denoted by p values with the notation: *p<0.05, **p<0.01, ***p<0.001, ****p<0.0001.

### DEFINITIONS

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

As used herein, the term “DISH” refers to Diffuse Idiopathic Skeletal Hyperostosis, also known as Forestier's disease, and is a skeletal disorder characterized by unusual, new bone formation (Resnick et al., Diffuse idiopathic skeletal hyperostosis (DISH): Forestier's disease with extraspinal manifestations, Radiology. 1975; 115:513-524). DISH causes ligaments and tendons in the body to calcify (harden) and/or appearance of new bone growth in peri-spinal and skeletal regions (ossified masses). These calcified areas can in some instances also form bone spurs (abnormal new bone growth) that can cause pain, stiffness, and decreased mobility. The new bone forms most often where ligaments and tendons join bone (entheseal area), but the relationship between entheseal calcifications and peri-spinal ossified masses is not understood. There is also a generalized hardening of bones and bone overgrowth (hyperostosis) (Pillai & Littlejohn, Metabolic Factors in Diffuse Idiopathic Skeletal Hyperostosis—A Review of Clinical Data, The Open Rheumatology Journal. 2014; 8:116-128). Another aspect of the disease is the formation of large, flowing osteophytes due to abnormal bone growth. These ossifications are mostly seen in the axial skeleton, of which the thoracic region is the main location. Also, peripheral entheses such as peripatellar ligaments, Achilles tendon insertion, plantar fascia, shoulders, olecranon and metacarpophalangeal joints can have calcifications as well. In some instances calcification is seen in hips, knees, ankles, feet, shoulders, hands, and ribs (Helfgott, Diffuse idiopathic skeletal hyperostosis (DISH). UpToDate. Jun. 7, 2017). The common characteristics and/or symptoms of DISH include focal and diffuse calcification and ossification of the anterior longitudinal ligament, calcification of paraspinal connective tissue and annulus fibrosus, degeneration of the peripheral annulus fibrosus fibers, anterolateral extensions of fibrous tissue, hypervascularity, chronic inflammatory cellular infiltration, periosteal new bone formation on the anterior surface of the vertebral bodies, ossification of metacarpophalangeal joints, pain in thoracic, lumbar, and/or cervical areas, radiculopathy, polyarticular pain, monoarticular synovitis, and/or dysphagia.

DISH manifests as buildup of calcium salts in the tendons and ligaments (calcification) and abnormal new bone growth (ossification) but the reason is unknown (Mader et al., Diffuse idiopathic skeletal hyperostosis: clinical features and pathogenic mechanisms, Nat Rev Rheumatol. December 2013; 9(12):741-50 & Nascimento et al. Diffuse idiopathic skeletal hyperostosis: A review, Surgical Neurology International. 2014; 5(Suppl 3):S122-S125). Common risk factors for developing DISH include but not limited to large waist circumference, BMI/Obesity, hyperinsulinemia, diabetes mellitus, hyperuricemia, dyslipidemia, hypertension, coronary artery disease and gout. DISH can be asymptomatic and in those cases, diagnosis is usually made on the basis of the radiographic images.

DISH is a progressive musculoskeletal disease associated with aging. The prevalence of DISH was 25% of males and 15% of females over the age of 50 in two large Midwestern hospital populations. In addition, up to 6.3% of the Japanese population, and 25% of patients with degenerative cervical myelopathy in North American develop progressive calcifications in the spinal posterior longitudinal ligament in a condition known as OPLL. Progressive calcium deposition in entheses results in canal stenosis and spinal cord compression from growing paraspinal masses. The resultant myelopathy is often severely painful and debilitating. Treatment usually consists of conservative chronic pain management with NSAIDs as there are no effective therapies preventing the progressive ossification responsible for the symptomatic course, and there is very little understanding of factors responsible for initiating and promoting the heterotopic mineralization. Decompression with surgery is used to provide short term relief in acute cases, but progression of entheses in OPLL occurs more rapidly after surgery (Laminoplasty) than in conservatively managed patients (70% vs. 24%, respectively), discouraging surgical intervention in all but severely symptomatic cases

A subject having DISH, as used herein, refers to a subject diagnosed with DISH. The diagnosis of DISH is based on radiological and/or clinical findings and defined by Resnick and Niwayana. Radiography of the thoracic and lumbar spine is the single most useful imaging modality in the diagnosis of DISH. Computed tomography (CT) scanning can be used to evaluate complications, such as fracture, or symptoms that can be caused by pressure effects on the trachea, oesophagus, and veins. This allows a differentiation of the entity towards ankylosing spondylitis or OPLL (Artner et al., Diffuse idiopathic skeletal hyperostosis: current aspects of diagnostics and therapy, 2012, Orthopade. 2012 November; 41(11):916-22; Olivieri et al., Diffuse idiopathic skeletal hyperostosis may give the typical postural abnormalities of advanced ankylosing spondylitis, Rheumatology 2007 Nov. 1; 46(11):1709-11). The presence of flowing calcifications and ossifications mainly along the anterolateral aspect (anterior longitudinal ligament) of at least 4 contiguous vertebrae (across 3 intervertebral disc spaces) with preserved disc height is indicative of the DISH. Spinal and extraspinal features (Radswiki & Baba, Diffuse idiopathic skeletal hyperostosis. Reference article, Radiopaedia.org) visible on radiograph and CT images of DISH patient is as follows:

### Non-Limiting Spinal Features:

flowing ossifications: florid, flowing ossification along the anterior or right anterolateral aspects of at least four contiguous vertebrae
disc spaces are usually well preserved
ankylosis is more common in the thoracic than cervical or lumbar spine
   frequently incomplete
   can have interdigitating areas of protruding disc material in the flowing ossifications
no sacroiliitis or facet joint ankylosis although sacroiliac joint anterior bridging, posterior bridging, entheseal bridging may be present 10

### Non-Limiting Extraspinal Features:

enthesopathy of the iliac crest, ischial tuberosities, and greater trochanters
spur formation in the appendicular skeleton (olecranon, calcaneum, patellar ligament) frequently present
‘whiskering’ enthesophytes

### Non-Limiting Clinical Features:

pain
a reduced range of motion
an increased risk of spinal fractures in some patients

In some instances, DISH becomes symptomatic, and the main clinical features include one or more of pain, stiffness and decreased mobility (range of motion), dysphagia (caused by compression of osteophytes), oesophagal obstruction, hoarseness, cervical myelopathy, atlantoaxial subluxation, spinal stenosis, ossification of the posterior, longitudinal ligament, spinal cord injury, dyspnea, foreign body sensation, neurologic manifestations due to compression of the spinal cord, hypercholesterinemia (resulting in cardiovascular comorbidities), and/or peripheral joint affection.

The “enthesis” is the site of attachment of tendons or ligaments to bone, and is structured in four zones: the dense fibrous connective tissue zone, populated by fibroblast-type cells (tenocytes) and composed of collagen types I and III and decorin; the unmineralized fibrocartilage, populated by fibrochondrocytes and composed of collagen types I and II and aggrecan; the mineralized fibrocartilage, populated by hypertrophic chondrocytes and composed of collagen types II and X and aggrecan; and the bone, populated by osteoblasts, osteocytes, and osteoclasts, and composed of collagen type I (Calejo et al., Enthesis tissue engineering: biological requirements meet at the interface, Tissue Eng Part B Rev. 2019; 25(4):330-356). The entheses thus represent a musculoskeletal structure that allows a smooth transition between two widely different tissues, the tendons or ligaments (compliant soft tissues) and bone (a stiff hard tissue) (Calejo et al., Enthesis tissue engineering: biological requirements meet at the interface, Tissue Eng Part B Rev. 2019; 25(4):330-356). An abrupt transition at this interface would lead to stress concentration between zones and increased risk of failure; conversely, a gradual transition in composition and structure over the enthesis alleviates stress concentrations. (Genin et al., Functional grading of mineral and collagen in the attachment of tendon to bone, Biophys J. 2009; 97(4):976-985). A gradual decrease in collagen fiber alignment and increase in mineral content is seen from the tendon toward the bone, creating a gradient of tissue stiffness (Genin et al., Functional grading of mineral and collagen in the attachment of tendon to bone, Biophys J. 2009; 97(4):976-985). A decrease in enthesis mineralization leads to decreased strength of this structure (Deymier et al., Micro-mechanical properties of the tendon-to-bone attachment, Acta Biomater. 2017; 1(56):25-35) whereas an animal model with expansion of the mineralized fibrocartilage also manifests decreased enthesis strength (Marinovich et al., The role of bone sialoprotein in the tendon-bone insertion, Matrix Biol. 2016; 52-54:325-338). Thus, mineralization must be appropriately regulated to achieve optimal mechanical properties of the entheses.

As used herein, “Ankylosing Spondylitis” refers to a type of arthritis characterized by long-term inflammation of the joints of the spine typically where the spine joins the pelvis. Areas affected may include other joints such as the shoulders or hips, eye and bowel problems may occur as well as back pain. Joint mobility in the affected areas generally worsens over time.

Although the cause of Ankylosing Spondylitis is unknown, it is believed to involve a combination of genetic and environmental factors. Many affected have a specific human leukocyte antigen known as the HLA-B27 antigen. The underlying mechanism is believed to be autoimmune or autoinflammatory. Diagnosis is typically based on the symptoms with support from medical imaging and blood tests. Ankylosing Spondylitis is a type of seronegative spondyloarthropathy, meaning that tests show no presence of rheumatoid factor (RF) antibodies. There is no known cure for Ankylosing Spondylitis. Treatments may include medication, exercise, physical therapy, and in rare cases surgery. Medications used include NSAIDs, steroids, DMARDs such as sulfasalazine, and biologic agents such as TNF inhibitors. Approximately 0.1% and 0.8% of all humans are affected with onset typically occurring in young adults. Males and females are equally affected; however, women are more likely than men to experience inflammation rather than fusion.

As used herein, “Spondyloarthritis” or “SpA” is characterized by inflammation in the axial skeleton (sacroiliitis, spondylitis), peripheral joints, and entheses. Extraskeletal manifestations can occur such as anterior uveitis, psoriasis, and inflammatory bowel disease. HLA-B27 is the major genetic risk factor. The entire group of SpA has a global prevalence of 0.1% to 1.9%, with variations between countries and ethnicities. Non-steroidal anti-inflammatory drugs (commonly called NSAIDs) offer symptom relief for most patients by reducing pain and swelling. Other medicines called biologics including anti-TNF drugs (TNF blockers) and anti-IL-17 drugs (IL-17 blockers) are effective in patients who do not respond well enough to NSAIDs.

“ENPP1 Deficiency” is characterized by a reduced level of ENPP1 enzymatic activity in serum and/or plasma of a subject. ENPP1 Deficiency is a rare, genetic disorder caused by inactivating mutations in the ENPP1 gene that encodes the ENPP1 enzyme. ENPP1 is an integral transmembrane protein whose extracellular domains carry pyrophosphatase and phosphodiesterase activities. ENPP1 converts extracellular ATP to inorganic pyrophosphate (PPi) and AMP.

“Enzymatically active” with respect to an ENPP1 polypeptide, or, as used herein, “enzymatic activity” with respect to an ENPP1 polypeptide, is defined as possessing ATP hydrolytic activity into AMP and PPi and/or AP3A hydrolysis to ATP. ENPP1 readily hydrolyze ATP into AMP and PPi. The steady-state Michaelis-Menten enzymatic constants of ENPP1 are determined using ATP as a substrate. ENPP1 can be demonstrated to cleave ATP by HPLC analysis of the enzymatic reaction, and the identity of the substrates and products of the reaction are confirmed by using ATP, AMP, and ADP standards. The ATP substrate degrades over time in the presence of ENPP1, with the accumulation of the enzymatic product AMP. Using varying concentrations of ATP substrate, the initial rate velocities for ENPP1 are derived in the presence of ATP, and the data is fit to a curve to derive the enzymatic rate constants. At physiologic pH, the kinetic rate constants of ENPP1 are Kₘ=2 μM and k_{cat}=3.4±0.4 s⁻¹.

As used herein the term “plasma pyrophosphate (PPi) levels” refers to the amount of pyrophosphate present in plasma of animals. In certain embodiments, animals include rat, mouse, cat, dog, human, cow and horse. It is necessary to measure PPi in the plasma rather than serum because of release from platelets. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications.

Typically, plasma PPi levels in healthy human subjects range from about 1 μM to about 3 μM, in some cases between 1-2 μM. A normal level of ENPP1 in plasma refers to the amount of ENPP1 protein required to maintain a normal level of plasma pyrophosphate (PPi) in a healthy subject. A normal level of PPi in healthy humans corresponds to 2-3 μM. Subjects who have a deficiency of ENPP1 exhibit low PPi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with GACI, the PPi levels are found to be less than 1 μM and in some cases are below a detectable level. In patients afflicted with PXE, the PPi levels are below 0.5 μM (Arterioscler Thromb Vasc Biol. 2014 September; 34(9):1985-9; Braddock et al., Nat Commun. 2015; 6: 10006).

As used herein, the term “pathological calcification” refers to the abnormal deposition of calcium salts in soft tissues, secretory and excretory passages of the body causing it to harden. There are two types: dystrophic calcification which occurs in dying and dead tissue, and metastatic calcification which is characterized by elevated extracellular levels of calcium (hypercalcemia), exceeding the homeostatic capacity of cells and tissues. Calcification can involve cells as well as extracellular matrix components such as collagen in basement membranes and elastic fibers in arterial walls. Some examples of tissues prone to calcification include: Gastric mucosa—the inner epithelial lining of the stomach, Kidneys and lungs, Cornea, Systemic arteries and Pulmonary veins.

As used herein, the term “pathological ossification” refers to a pathological condition in which bone arises in tissues not in the osseous system, or in connective tissues usually not manifesting osteogenic properties. Ossification is classified into three types depending on the nature of the tissue or organ being affected: endochondral ossification is ossification that occurs in and replaces cartilage; intramembranous ossification is ossification of bone that occurs in and replaces connective tissue; metaplastic ossification the development of bony substance in normally soft body structures; called also heterotrophic ossification.

A “deficiency” of ENPP1 refers to a condition in which the subject has less than or equal to 5%-10% of normal levels of ENPP1 in blood plasma. Normal levels of ENPP1 in healthy human subjects is approximately between 10 to 30 ng/ml (Am J Pathol. 2001 February; 158(2): 543-554).

“Ectopic calcification” refers to a condition characterized by a pathologic deposition of calcium salts in tissues or bone growth in soft tissues.

“Ectopic calcification of soft tissue” refers to inappropriate biomineralization, typically composed of calcium phosphate, hydroxyapatite, calcium oxalates, and octacalcium phosphates occurring in soft tissues leading to loss of hardening of soft tissues. “Arterial calcification” refers to ectopic calcification that occurs in arteries and heart valves leading to hardening and or narrowing of arteries. Calcification in arteries is correlated with atherosclerotic plaque burden and increased risk of myocardial infarction, increased ischemic episodes in peripheral vascular disease, and increased risk of dissection following angioplasty.

“Venous calcification” refers to ectopic calcification that occurs in veins that reduces the elasticity of the veins and restricts blood flow which can then lead to increase in blood pressure and coronary defects

“Vascular calcification” refers to the pathological deposition of mineral in the vascular system. It has a variety of forms, including intimal calcification and medial calcification, but can also be found in the valves of the heart. Vascular calcification is associated with atherosclerosis, diabetes, certain heredity conditions, and kidney disease, especially CKD. Patients with vascular calcification are at higher risk for adverse cardiovascular events. Vascular calcification affects a wide variety of patients. Idiopathic infantile arterial calcification is a rare form of vascular calcification where the arteries of neonates calcify.

“Brain calcification” (BC) refers to a nonspecific neuropathology wherein deposition of calcium and other mineral in blood vessel walls and tissue parenchyma occurs leading to neuronal death and gliosis. Brain calcification is often associated with various chronic and acute brain disorders including Down's syndrome, Lewy body disease, Alzheimer's disease, Parkinson's disease, vascular dementia, brain tumors, and/or various endocrinologic conditions.

Calcification of heart tissue refers to accumulation of deposits of calcium (possibly including other minerals) in tissues of the heart, such as aorta tissue and coronary tissue.

“Mineral bone disorders (MBD)” as used herein refers to a disorder characterized by abnormal hormone levels cause calcium and phosphorus levels in a person's blood to be out of balance. Mineral and bone disorder commonly occurs in people with CKD and affects most people with kidney failure receiving dialysis.

As used herein, the term “early onset osteoporosis” refers to beginning stages of osteoporosis commonly characterized by back pain, stooped posture, and/or slow loss of bone mass. Common causes include a low-calcium diet, smoking, age-related hormone changes.

“Osteopenia” is a bone condition characterized by decreased bone density, which leads to bone weakening and an increased risk of bone fracture. Osteomalacia is a bone disorder characterized by decreased mineralization of newly formed bone. Osteomalacia is caused by severe vitamin D deficiency (which can be nutritional or caused by a hereditary syndrome) and by conditions that cause very low blood phosphate levels. Both osteomalacia and osteopenia increase the risk of breaking a bone. Symptoms of osteomalacia include bone pain and muscle weakness, bone tenderness, difficulty walking, and muscle spasms.

“Age related osteopenia” as used herein refers to a condition in which bone mineral density is lower than normal. Generally, patients with osteopenia have a bone mineral density T-score of between −1.0 and −2.5. Osteopenia if left untreated progresses into Osteoporosis where bones become brittle and are extremely prone to fracture.

“Ossification of posterior longitudinal ligament (OPLL)” as used herein refers to a hyperostotic (excessive bone growth) condition that results in ectopic calcification of the posterior longitudinal ligament. The posterior longitudinal ligament connects and stabilizes the bones of the spinal column. The thickened or calcified ligament may compress the spinal cord, producing myelopathy. Symptoms of myelopathy include difficulty walking and difficulty with bowel and bladder control. OPLL may also cause radiculopathy, or compression of a nerve root. Symptoms of cervical radiculopathy include pain, tingling, or numbness in the neck, shoulder, arm, or hand. OPLL is distinct from DISH, as ossification occurs only in the posterior longitudinal ligament for OPLL, unlike DISH where ossification also occurs in the thoracic region and the anterior longitudinal ligament.

Clinical symptoms and signs caused by OPLL are categorized as: (1) myelopathy, or a spinal cord lesion with motor and sensory disturbance of the upper and lower limbs, spasticity, and bladder dysfunction; (2) cervical radiculopathy, with pain and sensory disturbance of the upper limbs; and (3) axial discomfort, with pain and stiffness around the neck. The most common symptoms in the early stages of OPLL include dysesthesia and tingling sensation in hands, and clumsiness. With the progression of neurologic deficits, lower extremity symptoms, such as gait disturbance may appear. OPLL is detected on lateral plain radiographs, and the diagnosis and morphological details of cervical OPLL have been clearly demonstrated by magnetic resonance imaging (MRI) and computed tomography (CT).

OPLL is prevalent in Americans with cervical myelopathy and even more extensively in the Asian population. Myelopathy and decreased mobility progressively worsen with age, and there are no effective measures which prevent the progression of the paraspinal ossifications responsible for myelopathy and stiffness. Current therapy is focused on symptomatic relief, and while surgery may be helpful in the short term, subsequent rapid progression of enthesopathy and recurrence of symptoms often complicates this approach.

Patients with rapidly progressive OPLL demonstrate elevations in circulating FGF23 (Kawaguchi, Y., et al., Serum biomarkers in patients with ossification of the posterior longitudinal ligament (OPLL): Inflammation in OPLL. PLoS One, 2017. 12(5): p. e0174881; Kawaguchi, Y., et al., Increase of the Serum FGF-23 in Ossification of the Posterior Longitudinal Ligament. Global Spine J, 2019. 9(5): p. 492-498), a finding central to the rare disorders X-linked hypophosphatemia (XLH) and autosomal recessive hypophosphatemic rickets (ARHR), both of which also exhibit enthesopathies similar to DISH and OPLL. These findings indicate a causative role for FGF23 (or resultant hypophosphatemia) in the pathogenesis of enthesopathy, however a mechanism by which elevated FGF23 and/or reduced phosphate may induce enthesopathy or spinal ossifications is unknown.

“Hereditary Hypophosphatemic Rickets” as used herein refers to a disorder related to low levels of phosphate in the blood (hypophosphatemia). Phosphate is a mineral that is essential for the normal formation of bones and teeth. Most commonly, it is caused by a mutation in the PHEX gene. Other genes that can be responsible for the condition include the CLCN5, DMP1, ENPP1, FGF23, and SLC34A3 genes. Other signs and symptoms of hereditary hypophosphatemic rickets can include premature fusion of the skull bones (craniosynostosis) and dental abnormalities. The disorder may also cause abnormal bone growth where ligaments and tendons attach to joints (enthesopathy). In adults, hypophosphatemia is characterized by a softening of the bones known as osteomalacia. Another rare type of the disorder is known as hereditary hypophosphatemic rickets with hypercalciuria (HHRH) wherein in addition to hypophosphatemia, this condition is characterized by the excretion of high levels of calcium in the urine (hypercalciuria).

“X-linked hypophosphatemia (XLH)”, as used herein, the term X-linked hypophosphatemia (XLH), also called X-linked dominant hypophosphatemic rickets, or X-linked Vitamin D-resistant rickets, is an X-linked dominant form of rickets (or osteomalacia) that differs from most cases of rickets in that vitamin D supplementation does not cure it. It can cause bone deformity including short stature and genu varum (bow leggedness). It is associated with a mutation in the PHEX gene sequence (Xp.22) and subsequent inactivity of the PHEX protein.

“Autosomal Recessive Hypophosphatemia Rickets type 2 (ARHR2)” as used herein refers to a hereditary renal phosphate-wasting disorder characterized by hypophosphatemia, rickets and/or osteomalacia and slow growth. Autosomal recessive hypophosphatemic rickets type 2 (ARHR2) is caused by homozygous loss-of-function mutation in the ENPP1 gene.

“Autosomal Dominant Hypophosphatemic Rickets (ADHR)” as used herein refers to a rare hereditary disease in which excessive loss of phosphate in the urine leads to poorly formed bones (rickets), bone pain, and tooth abscesses. ADHR is caused by a mutation in the fibroblast growth factor 23 (FGF23). ADHR is characterized by impaired mineralization of bone, rickets and/or osteomalacia, suppressed levels of calcitriol (1,25-dihydroxyvitamin D3), renal phosphate wasting, and low serum phosphate. Mutations in FGF23 render the protein more stable and uncleavable by proteases resulting in enhanced bioactivity of FGF23. The enhanced activity of FGF23 mutants reduce expression of sodium-phosphate co-transporters, NPT2a and NPT2c, on the apical surface of proximal renal tubule cells, resulting in renal phosphate wasting.

“Hypophosphatemic rickets” (previously called vitamin D-resistant rickets) is a disorder in which the bones become painfully soft and bend easily, due to low levels of phosphate in the blood. Symptoms may include bowing of the legs and other bone deformities; bone pain; joint pain; poor bone growth; and short stature. In some affected babies, the space between the skull bones closes too soon leading to craniosynostosis. Most patients display abnormality of calcium-phosphate metabolism, abnormality of dental enamel, delayed eruption of teeth and long, narrow head (dolichocephaly).

As used herein, the term “ENPP1 haploinsufficiency” refers to a genetic condition wherein one copy of ENPP1 gene is inactivated or deleted and the remaining functional copy of the ENPP1 gene is not adequate to produce the needed gene product to preserve normal function. As a result of ENPP1 haploinsufficiency can, but does not necessarily have to, manifest in the form of low PPi levels and pathological calcifications. Diseases like DISH or early onset of osteoporosis can in certain embodiments be associated with, and/or caused by, ENPP1 haploinsufficiency.

For most genes, a single copy is enough to support normal growth and development of diploid organisms, but a small subset of genes known as haploinsufficient (HI) genes exhibit extreme sensitivity to decreased gene dosage. Given the relatively high frequency of gene-inactivating mutations over the lifespan of an organism, and cell-to-cell variability in gene expression, haploinsufficiency represents a significant barrier to organismal fitness. Haploinsufficiency in genetics describes a model of dominant gene action in diploid organisms, in which a single copy of the wild-type allele at a locus in heterozygous combination with a variant allele is insufficient to produce the wild-type phenotype. Haploinsufficiency may arise from a de novo or inherited loss-of-function mutation in the variant allele, such that it produces little or no gene product.

For example, N179S mutation in ENPP1 has been found to result in the loss of function of ENPP1 protein. The presence of a single copy of the mutated ENPP1 gene having N179S mutation results in decreased PPi production. The invention discloses that the N179S mutation is found in certain patients with DISH or early onset of osteoporosis and can in certain embodiments serve as a genetic marker for the presence or future development and/or progression of DISH or osteoporosis.

Likewise, Y451C mutation in ENPP1 has been found to result in the loss of function of ENPP1 protein. The presence of a single copy of the mutated ENPP1 gene having Y451C mutation results in decreased PPi production. The invention discloses that the Y451C mutation is found in certain patients with DISH or early onset of osteoporosis and can in certain embodiments serve as a genetic marker for the presence or future development and/or progression of DISH or osteoporosis.

“Pre-treatment”, as used herein, means treatment prior to commencement of a treatment method described herein.

The term “subject”, as used herein, refers to an individual, such as a mammal, such as a human, a non-human primate (e.g. chimpanzees and other apes and monkey species), a farm animal (e.g. birds, fish, cattle, sheep, pigs, goats, and horses), a domestic mammal (e.g. dogs and cats), or a laboratory animal (e.g. rodents, such as mice, rats and guinea pigs). The term includes a subject of any age or sex. In another embodiment the subject is a mammal, preferably a human.

A disease or disorder is “alleviated” if the severity of a symptom of the disease or disorder, the frequency with which such a symptom is experienced by a patient, or both, is reduced.

As used herein, the terms “alteration,” “defect,” “variation,” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “ENPP” or “NPP” refers to ectonucleotide pyrophosphatase/phosphodiesterase.

As used herein, the term “ENPP1 protein” or “ENPP1 polypeptide” refers to ectonucleotide pyrophosphatase/phosphodiesterase-1 protein encoded by the ENPP1 gene. The encoded protein is a type II transmembrane glycoprotein and cleaves a variety of substrates, including phosphodiester bonds of nucleotides and nucleotide sugars and pyrophosphate bonds of nucleotides and nucleotide sugars. ENPP1 protein has a transmembrane domain and soluble extracellular domain. The extracellular domain is further subdivided into somatomedin B domain, catalytic domain (residues 186 to 586 of SEQ ID NO: 1) and the nuclease domain (residues 524 to 885 of SEQ ID NO: 1). The sequence and structure of wild-type ENPP1 is described in detail in PCT Application Publication No. WO 2014/126965 to Braddock et al., which is incorporated herein in its entirety by reference.

Mammal ENPP1 polypeptides, mutants, or mutant fragments thereof, have been previously disclosed in International PCT Application Publications No. WO/2014/126965 to Braddock et al., WO/2016/187408 to Braddock et al., WO/2017/087936 to Braddock et al., and WO2018/027024 to Braddock et al., all of which are incorporated by reference in their entireties herein.

As used herein, the term “ENPP1 precursor protein” refers to ENPP1 with its signal peptide sequence at the ENPP1 N-terminus. Upon proteolysis, the signal sequence (in certain non-limiting embodiments, denoted by W in the compound of formula (I)) is cleaved from ENPP1 to provide the ENPP1 protein. Signal peptide sequences useful within the invention include, but are not limited to, albumin signal sequence, azurocidin signal sequence, ENPP1 signal peptide sequence, ENPP2 signal peptide sequence, ENPP7 signal peptide sequence, and/or ENPP5 signal peptide sequence.

As used herein, the term “ENPP1-Fc construct” refers to ENPP1 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “Fc” refers to a human IgG (immunoglobulin) Fc domain. Subtypes of IgG such as IgG1, IgG2, IgG3, and IgG4 are contemplated for use as Fc domains.

As used herein, the “Fc region or Fc polypeptide” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, the term “patient,” “individual” or “subject” refers to a human.

As used herein, the term “pharmaceutical composition” or “composition” refers to a mixture of at least one compound useful within the invention with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained; for example, phosphate-buffered saline (PBS).

As used herein the term “plasma pyrophosphate (PPi) levels” refers to the amount of pyrophosphate present in plasma of animals. In certain embodiments, animals include rat, mouse, cat, dog, human, cow and horse. It is necessary to measure PPi in plasma rather than serum because of release from platelets. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications. Typically, normal PPi levels in healthy subjects range from about 1 μM to about 3 μM, in some cases between 1-2 μM. In some cases, a “low” level of PPi refers to a condition in which the subject has less than or equal to 2%-5% of normal levels of plasma pyrophosphate (PPi) (Arthritis and Rheumatism, Vol. 22, No. 8 (August 1979)).

Subjects who have defective ENPP1 expression tend to exhibit low PPi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with GACI, the PPi levels are found to be less than 1 μM and in some cases are below a detectable level. In patients afflicted with PXE, the PPi levels are below 0.5 μM (Arterioscler Thromb Vasc Biol. 2014 September; 34(9):1985-9; Braddock et al., Nat Commun. 2015; 6: 10006).

As used herein, the term “polypeptide” refers to a polymer composed of amino acid residues, related naturally occurring structural variants, and synthetic non-naturally occurring analogs thereof linked via peptide bonds.

As used herein, the term “PPi” refers to pyrophosphate.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein, the term “treatment” or “treating” is defined as the application or administration of a therapeutic agent, i.e., a compound useful within the invention (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve, affect, and/or prevent and/or minimize progression of the disease or disorder and/or the symptoms of the disease or disorder. Such treatments may be specifically tailored or modified, based on knowledge obtained from the field of pharmacogenomics.

The terms “prevent,” “preventing,” and “prevention”, as used herein, refer to inhibiting the inception or decreasing the occurrence of a disease in a subject. Prevention may be complete (e.g. the total absence of pathological cells in a subject) or partial. Prevention also refers to a reduced susceptibility to a clinical condition. In certain embodiments, “preventing” comprises preventing onset of a disease or disorder.

As used herein, the term “wild-type” refers to a gene or gene product isolated from a naturally occurring source. A wild-type gene is most frequently observed in a population and is thus arbitrarily designed the “normal” or “wild-type” form of the human ENPP1 genes. In contrast, the term “functionally equivalent” refers to a ENPP1 gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

The term “functional equivalent variant”, as used herein, relates to a polypeptide substantially homologous to the sequences of ENPP1 (defined above) and that preserves the enzymatic and biological activities of ENPP1. Methods for determining whether a variant preserves the biological activity of the native ENPP1 are widely known to the skilled person and include any of the assays used in the experimental part of the application. Particularly, functionally equivalent variants of ENPP1 delivered by viral vectors is encompassed by the present invention.

The functionally equivalent variants of ENPP1 are polypeptides substantially homologous to the native ENPP1. The expression “substantially homologous” relates to a protein sequence when the protein sequence has a degree of identity with respect to the ENPP1 sequences described above of at least 80%, at least 85%, at least 90%, at least 91%, at least 92%, at least 93%, at least 94%, at least 95%, at least 96%, at least 97%, at least 98% or at least 99% respectively.

The degree of identity between two polypeptides is determined using computer algorithms and methods that are widely known for the persons skilled in the art. The identity between two amino acid sequences is preferably determined by using the BLASTP algorithm (BLAST Manual, Altschul et al., NCBI NLM NIH Bethesda, Md. 20894, Altschul et al., J. Mol. Biol. 215: 403-410 (1990)), though other similar algorithms can also be used. BLAST and BLAST 2.0 are used, with the parameters described herein, to determine percent sequence identity. Software for performing BLAST analyses is publicly available through the National Center for Biotechnology Information.

“Functionally equivalent variants” of ENPP1 may be obtained by replacing nucleotides within the polynucleotide accounting for codon preference in the host cell that is to be used to produce the ENPP1 respectively. Such “codon optimization” can be determined via computer algorithms which incorporate codon frequency tables such as “Human high.cod” for codon preference as provided by the University of Wisconsin Package Version 9.0, Genetics Computer Group, Madison, Wis.

“About” as used herein when referring to a measurable value such as an amount, a temporal duration, and the like, is meant to encompass variations of ±10% or ±5%, in certain embodiments ±1-5%, in certain embodiments ±5%, in certain embodiments ±4%, in certain embodiments ±4%, in certain embodiments ±3%, in certain embodiments ±2%, and in certain embodiments ±1% from the specified value (0.2 mg/kg or 0.6 mg/kg or 1.8 mg/kg), as such variations are appropriate to perform the disclosed methods.

The disclosure provides a representative example of protein sequences. The protein sequences described can be converted into nucleic acid sequences by performing revere translation and codon optimization. There are several tools available in art such as Expasy (https://www.expasy.org/) and bioinformatics servers (www dot bioinformatics dot org) that enable such conversions

Ranges: throughout this disclosure, various aspects according to the invention can be presented in a range format. It should be understood that the description in range format is merely for convenience and brevity and should not be construed as an inflexible limitation on the scope according to the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### DETAILED DESCRIPTION

### 1. ENPP1 Agent

An ENPP1 agent is an ENPP1 polypeptide. ENPP1 polypeptides disclosed herein include, but are not limited to, naturally occurring polypeptides of the ENPP1 family as well as any variants thereof (including mutants, fragments, fusions, and peptidomimetic forms) that retain a biological activity, such as but not limited to ENPP1's catalytic activity.

The ENPP1 agent can be represented in the form of a formula shown below:

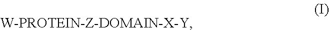

wherein in (I):
W is absent or comprises a signal sequence which allows for export of the compound into extracellular space;
PROTEIN comprises the catalytic region of ENPP1;
DOMAIN is absent or at least one selected from the group consisting of a human IgG Fc domain (Fc), human serum albumin protein (ALB), and a biologically active fragment thereof;
X and Z are independently absent or a polypeptide comprising 1-20 amino acids; and
Y is absent or a sequence selected from the “bone targeting” sequence group consisting of: Dₘ (SEQ ID NO:11), (DSS)ₙ(SEQ ID NO:12), (ESS)ₙ(SEQ ID NO:13), (RQQ)ₙ (SEQ ID NO:14), (KR)₁ (SEQ ID NO:15), Rₘ (SEQ ID NO:16), DSSSEEKFLRRIGRFG (SEQ ID NO:17), EEEEEEEPRGDT (SEQ ID NO: 18), APWHLSSQYSRT (SEQ ID NO: 19), STLPIPHEFSRE (SEQ ID NO: 20), VTKHLNQISQSY (SEQ ID NO: 116), and Eₘ (SEQ ID NO: 117),
   wherein m is independently an integer ranging from 1 to 15, and
   wherein n is independently an integer ranging from 1 to 10.

In some embodiments, W is absent. In some embodiments, W comprises a signal sequence which allows for export of the compound into extracellular space.

In some embodiments, the ENPP1 agent comprises a bone targeting domain. In some embodiments, the bone targeting domain is negatively charged sequence of amino acids. In some embodiments, the negatively charged bone targeting domain is polyaspartic acid.

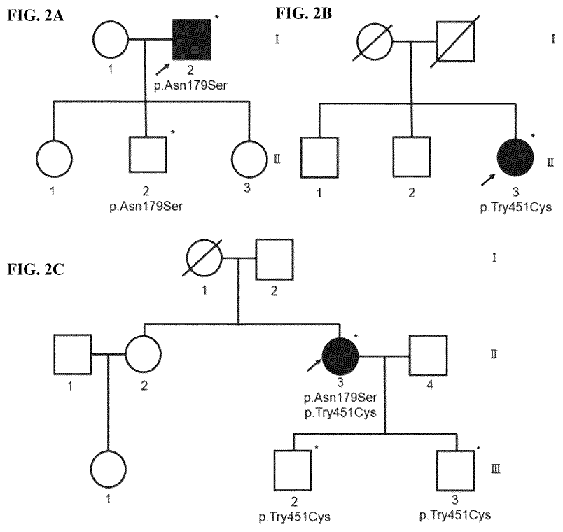

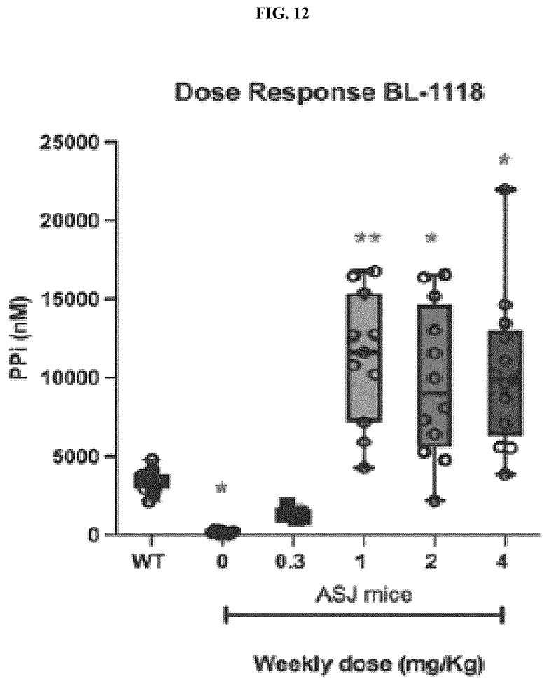

The terms “ENPP1” or “ENPP1 polypeptide” refers to ectonucleotide pyrophosphatase/phosphodiesterase 1 proteins (NPP1/ENPP1/PC-1) and ENPP1-related proteins, derived from any species. ENPP1 protein comprises a type II transmembrane glycoprotein that forms a homodimer. Each monomer of the ENPP1 protein comprises a short intracellular N-terminal domain involved in targeting to the plasma membrane, a transmembrane domain, and a large extracellular region comprising several domains. The large extracellular region comprises SMB1 and SMB2 domains, which have been reported to take part in ENPP1 dimerization (Gijsbers et al., Biochem. J. 371; 2003: 321-330). Specifically, the SMB domains contain eight cysteine residues, each arranged in four disulfide bonds, and have been shown to mediate ENPP1 homodimerization through covalent cystine inter- and intramolecular bonds. The protein cleaves a variety of substrates, including phosphodiester bonds of nucleotides and nucleotide sugars and pyrophosphate bonds of nucleotides and nucleotide sugars. ENPP1 protein functions to hydrolyze nucleoside 5′ triphosphatase to either corresponding monophosphates and also hydrolyzes diadenosine polyphosphates. ENPP1 proteins play a role in purinergic signaling which is involved in the regulation of cardiovascular, neurological, immunological, musculoskeletal, hormonal, and hematological functions. An exemplary amino acid sequence of the human ENPP1 precursor protein (NCBI accession NP_006199) is shown in FIG. 11 (SEQ ID NO: 1). The human ENPP1 precursor protein includes an endogenous ENPP1 signal peptide sequence at the ENPP1 N-terminus. Numbering of amino acids for all ENPP1-related polypeptides described herein is based on the numbering of the human ENPP1 precursor protein sequence provided in FIG. 2 unless specifically designated otherwise. In certain embodiments, the ENPP1 precursor protein further comprises an endogenous or heterologous signal peptide sequence. Upon proteolysis, the signal peptide sequence is cleaved from the ENPP1 precursor protein to provide the mature ENPP1 protein. See, e.g., Jansen et al. J Cell Sci. 2005; 118(Pt 14):3081-9. Exemplary signal peptide sequences that can be used with the polypeptides disclosed herein include, but are not limited to, ENPP1 signal peptide sequence, ENPP2 signal peptide sequence, ENPP7 signal peptide sequence, and/or ENPP5 signal peptide sequence. A non-limiting processed (mature) extracellular ENPP1 polypeptide sequence is shown in SEQ ID NO:2.

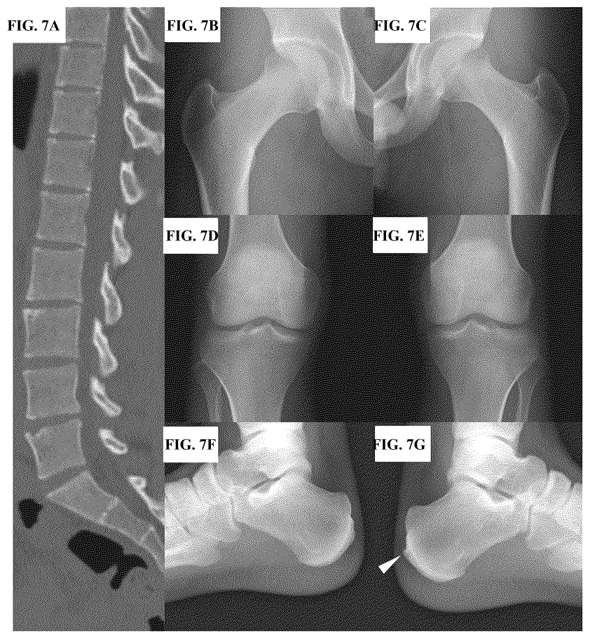

It is generally known in the art that ENPP1 is well-conserved among vertebrates, with large stretches of the extracellular domain substantially conserved. For example, FIGS. 7A-7B depict a multi-sequence alignment of a human ENPP1 extracellular domain compared to various ENPP1 orthologs. ENPP1 binding to various nucleotide triphosphates (e.g., ATP, UTP, GTP, TTP, and CTP), pNP-TMP, 3′,5′-cAMP, and 2′-3′-cGAMP is also highly conserved (see, e.g., Kato et al., Proc Natl Acad Sci USA. 2012; 109(42):16876-81 and Mackenzie et al. Bone. 2012; 51(5):961-8). Accordingly, from these alignments, it is possible to predict key amino acid positions with the extracellular domain that are important for normal ENPP1 activities as well as to predict amino acid positions that are likely to be tolerant to substitution without significantly altering normal ENPP1 activities. Therefore, an enzymatically active, human ENPP1 polypeptide useful in accordance with the presently disclosed compositions may include one or more amino acids at corresponding positions from the sequence of another vertebrate ENPP1, or may include a residue that is similar to that in the human or other vertebrate sequences. Substitutions of one or more amino acids at corresponding positions may include conservative variations or substitutions that are not likely to change the shape of the polypeptide chain or alter normal ENPP1 activities. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine. For example, ENPP1 polypeptides include polypeptides derived from the sequence of any known ENPP1 polypeptide having a sequence at least about 80% identical to the sequence of an ENPP1 polypeptide, and preferably at least 85%, 90%, 91%, 92%, 93%, 94%, 95%, 96%, 97%, 98%, 99% or greater identity.

### 2. Enzymatic Activity of ENPP1

ENPP1 proteins have been characterized in the art in terms of structural and biological characteristics. In certain embodiments, soluble ENPP1 proteins disclosed herein comprise pyrophosphatase and/or phosphodiesterase activity. For instance, in some embodiments, the ENPP1 protein binds nucleotide triphosphates (e.g., ATP, UTP, GTP, TTP, and CTP), pNP-TMP, 3′,5′-cAMP, and 2′-3′-cGAMP; and converts nucleotide triphosphates into inorganic pyrophosphate [see, e.g., Kato et al., Proc Natl Acad Sci USA. 2012; 109(42):16876-81; Li, et al., Nat Chem Biol. 2014; 10(12):1043-8; Jansen et al., Structure. 2012; 20(11):1948-59; and Onyedibe et al., Molecules. 2019; 24(22)].

“Enzymatically active” or “biologically active” ENPP1 polypeptides exhibit pyrophosphatase and/or phosphodiesterase activity (e.g., is capable of binding and/or hydrolyzing ATP into AMP and PPi and/or AP3a into ATP). For example, the pyrophosphatase/phosphodiesterase domain of an ENPP1 protein hydrolyzes extracellular nucleotide triphosphates to produce inorganic pyrophosphates (PPi) and is generally soluble. This activity can be measured using a pNP-TMP assay as previously described (Saunders et al., 2008, Mol. Cancer Ther. 7(10):3352-62; Albright et al., 2015, Nat Comm. 6:10006). In certain embodiments, the soluble ENPP1 polypeptide has a k_{cat} value for the substrate ATP greater than or equal to about 3.4 (±0.4) s⁻¹ enzyme⁻¹, wherein the k_{cat} is determined by measuring the rate of hydrolysis of ATP for the polypeptide. In certain embodiments, the soluble ENPP1 polypeptide has a K_{M} value for the substrate ATP less than or equal to about 2 μM, wherein the K_{M} is determined by measuring the rate of hydrolysis of ATP for the polypeptide. In addition to the teachings herein, these references provide ample guidance for how to generate soluble ENPP1 proteins that retain one or more biological activities (e.g., conversion of nucleotides into inorganic pyrophosphate).

### 3. Soluble ENPP1

In one embodiment, the disclosure relates to ENPP1 agents, such as but not limited to ENPP1 polypeptides. As described herein, the term soluble ENPP1 polypeptide includes any naturally occurring extracellular domain of an ENPP1 protein as well as any variants thereof (including mutants, fragments and peptidomimetic forms) that retain a biological activity (e.g., enzymatically active). Examples of soluble ENPP1 polypeptides include, for example, an ENPP1 extracellular domain (SEQ ID NO: 2). In certain embodiments, the soluble ENPP1 polypeptides further comprise a signal sequence in addition to the extracellular domain of an ENPP1 polypeptide. Exemplary signal sequences include the native signal sequence of an ENPP1 polypeptide, or a signal sequence from another protein, such as a hENPP7 signal sequence. Examples of variant soluble ENPP1 polypeptides are provided in International Patent Application Publication Nos. WO 2012/125182, WO 2014/126965, WO 2016/187408, WO 2018/027024, WO 2020206302 and WO 2020/047520. the contents of all of which are incorporated herein by reference in their entirety.

### 4. ENPP1 Fusion Proteins

In some embodiments, the ENPP1 polypeptide is a fusion protein comprising an ENPP1 polypeptide domain and one or more heterologous protein portions (i.e., polypeptide domains heterologous to ENPP1). An amino acid sequence is understood to be heterologous to ENPP1 if it is not uniquely found in the form of ENPP1 represented by SEQ ID NO: 1. In some embodiments, the heterologous protein portion comprises an Fc domain of an immunoglobulin. In some embodiments, the Fc domain of the immunoglobulin is an Fc domain of an IgG1 immunoglobulin. In certain embodiments, the soluble ENPP1 polypeptide is C-terminally fused to the Fc domain of human immunoglobulin 1 (IgG1), human immunoglobulin 2 (IgG2), human immunoglobulin 3 (IgG3), and/or human immunoglobulin 4 (IgG4). In other embodiments, the soluble ENPP1 polypeptide is N-terminally fused to the Fc domain of human immunoglobulin 1 (IgG1), human immunoglobulin 2 (IgG2), human immunoglobulin 3 (IgG3), and/or human immunoglobulin 4 (IgG4). In some embodiments, the presence of an Fc domain improves half-life, solubility, reduces immunogenicity, and increases the activity of the soluble ENPP1 polypeptide. In certain embodiments, portions of the native human IgG proteins (IgG1, IgG2, IgG3, and IgG4), may be used for the Fc portion (e.g., ENPP1-Fc). For instance, the present disclosure provides fusion proteins comprising ENPP1 fused to a polypeptide comprising a constant domain of an immunoglobulin, such as a CH1, CH2, or CH3 domain derived from human IgG1, IgG2, IgG3, and/or IgG4. The Fc fragment may comprise regions of the native IgG such as the hinge region (residues 216-230 of human IgG1, according to the Rabat numbering system), the entire second constant domain CH2 (residues 231-340), and the third constant domain CH3 (residues 341-447). As used herein, the term “ENPP1-Fc construct” refers to a soluble form of ENPP1 (e.g., the extracellular domain of an ENPP1 polypeptide) recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

An example of an amino acid sequence that may be used for the Fc portion of human IgG1 (G1Fc) is SEQ ID NO: 6 (Table 1). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 6.

In some embodiments, the heterologous protein portion comprises one or more domains selected from the group consisting of polyhistidine, FLAG tag, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ENPP1 polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation.

### 5. Linkers

In some embodiments, the ENPP1 fusion protein further comprises a linker (Z) positioned between the ENPP1 polypeptide domain and the one or more heterologous protein portions (e.g., an Fc immunoglobulin domain). In certain embodiments, the soluble ENPP1 polypeptide is directly or indirectly fused to the Fc domain. In some embodiments, the soluble ENPP1 fusion protein comprises a linker between the Fc domain and the ENPP1 polypeptide. In some embodiments, a linker can be an amino acid spacer including 1-200 amino acids. Suitable peptide spacers are known in the art, and include, for example, peptide linkers containing flexible amino acid residues such as glycine, alanine, and serine.

In some embodiments, the linker comprises a polyglycine linker or a Gly-Ser linker. In some embodiments, a spacer can contain motifs, e.g., multiple or repeating motifs, of GA (SEQ ID NO: 21), GS (SEQ ID NO: 22), GG (SEQ ID NO: 23), GGA (SEQ ID NO: 24), GGS (SEQ ID NO: 25), GGG (SEQ ID NO: 26), GGGA (SEQ ID NO: 27), GGGS (SEQ ID NO: 28), GGGG (SEQ ID NO: 29), GGGGA (SEQ ID NO: 30), GGGGS (SEQ ID NO: 31), GGGGG (SEQ ID NO: 32), GGAG (SEQ ID NO: 33), GGSG (SEQ ID NO: 34), AGGG (SEQ ID NO: 35), SGGGG (SEQ ID NO: 36), or SGGG (SEQ ID NO: 37). In some embodiments, a spacer can contain 2 to 12 amino acids including motifs of GA or GS, e.g., GA, GS, GAGA (SEQ ID NO: 38), GSGS (SEQ ID NO: 39), GAGAGA (SEQ ID NO: 40), GSGSGS (SEQ ID NO: 41), GAGAGAGA (SEQ ID NO: 42), GSGSGSGS (SEQ ID NO: 43), GAGAGAGAGA (SEQ ID NO: 44), GSGSGSGSGS (SEQ ID NO: 45), GAGAGAGAGAGA (SEQ ID NO: 46), and GSGSGSGSGSGS (SEQ ID NO: 47). In some embodiments, a spacer can contain 3 to 12 amino acids including motifs of GGA or GGS, e.g., GGA, GGS, GGAGGA (SEQ ID NO: 48), GGSGGS (SEQ ID NO: 49), GGAGGAGGA (SEQ ID NO: 50), GGSGGSGGS (SEQ ID NO: 51), GGAGGAGGAGGA (SEQ ID NO: 52), and GGSGGSGGSGGS (SEQ ID NO: 53). In yet some embodiments, a spacer can contain 4 to 12 amino acids including motifs of GGAG (SEQ ID NO: 54), GGSG (SEQ ID NO: 55), e.g., GGAG (SEQ ID NO: 56), GGSG (SEQ ID NO: 57), GGAGGGAG (SEQ ID NO: 58), GGSGGGSG (SEQ ID NO: 59), GGAGGGAGGGAG (SEQ ID NO: 60), and GGSGGGSGGGSG (SEQ ID NO: 61). In some embodiments, a spacer can contain motifs of GGGGA (SEQ ID NO: 62) or GGGGS (SEQ ID NO: 63), e.g., GGGGAGGGGAGGGGA (SEQ ID NO: 64) and GGGGSGGGGSGGGGS (SEQ ID NO: 65). In some embodiments of the invention, an amino acid spacer between a heterologous protein portion (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a soluble ENPP1 polypeptide may be GGG, GGGA (SEQ ID NO: 27), GGGG (SEQ ID NO: 29), GGGAG (SEQ ID NO: 66), GGGAGG (SEQ ID NO: 67), or GGGAGGG (SEQ ID NO: 68).

In some embodiments, a spacer can also contain amino acids other than glycine, alanine, and serine, e.g., LIN (SEQ ID NO: 69), TGGGG (SEQ ID NO: 70), AAAL (SEQ ID NO: 71), AAAK (SEQ ID NO: 72), AAAR (SEQ ID NO: 73), EGKSSGSGSESKST (SEQ ID NO: 74), GSAGSAAGSGEF (SEQ ID NO: 75), AEAAAKEAAAKA (SEQ ID NO: 76), KESGSVSSEQLAQFRSLD (SEQ ID NO: 77), GENLYFQSGG (SEQ ID NO: 78), SACYCELS (SEQ ID NO: 79), RSIAT (SEQ ID NO: 80), RPACKIPNDLKQKVMNH (SEQ ID NO: 81), GGSAGGSGSGSSGGSSGASGTGTAGGTGSGSGTGSG (SEQ ID NO: 82), AAANSSIDLISVPVDSR (SEQ ID NO: 83), GGSGGGSEGGGSEGGGSEGGGSEGGGSEGGGSGGGS (SEQ ID NO: 84), NSS (SEQ ID NO: 87), ESS (SEQ ID NO: 88), RQQ (SEQ ID NO: 89), KR (SEQ ID NO: 90), (R)ₘ; m=0-15 (SEQ ID NO: 91), DSSSEEKFLRRIGRFG (SEQ ID NO: 92), EEEEEEEPRGDT (SEQ ID NO: 93), APWHLSSQYSRT (SEQ ID NO: 94), STLPIPHEFSRE (SEQ ID NO: 95), VTKHLNQISQSY (SEQ ID NO: 96), (E)ₘ; m=1-15 (SEQ ID NO: 97), RSGSGGS (SEQ ID NO: 98), (D)ₘ; m=1-15 (SEQ ID NO: 99), LVIMSLGLGLGLGLRK (SEQ ID NO: 100), VIMSLGLGLGLGLRK (SEQ ID NO: 101), IMSLGLGLGLGLRK (SEQ ID NO: 102), MSLGLGLGLGLRK (SEQ ID NO: 103), SLGLGLGLGLRK (SEQ ID NO: 104), LGLGLGLGLRK (SEQ ID NO: 105), GLGLGLGLRK (SEQ ID NO: 106), LGLGLGLRK (SEQ ID NO: 107), GLGLGLRK (SEQ ID NO: 108), LGLGLRK (SEQ ID NO: 109), GLGLRK (SEQ ID NO: 110), LGLRK (SEQ ID NO: 111), GLRK (SEQ ID NO: 112), LRK (SEQ ID NO: 113), RK (SEQ ID NO: 114), or (K)ₘ; m=1-15 (SEQ ID NO: 115). In some embodiments, a spacer can contain motifs, e.g., multiple or repeating motifs, of EAAAK (SEQ ID NO: 85). In some embodiments, a spacer can contain motifs, e.g., multiple or repeating motifs, of praline-rich sequences such as (XP)n, in which X may be any amino acid (e.g., A, K, or E) and n is from 1-5, and PAPAP(SEQ ID NO: 86).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, different elements of the fusion proteins (e.g., immunoglobulin Fc fusion proteins) may be arranged in any manner that is consistent with desired functionality. For example, a soluble ENPP1 polypeptide domain may be placed C-terminal to a heterologous protein portion, or alternatively, a heterologous protein portion may be placed C-terminal to a soluble ENPP1 polypeptide domain. The soluble ENPP1 polypeptide domain and the heterologous protein portion may be directly or indirectly linked in a fusion protein, and additional domains or amino acid sequences may be included C- or N-terminal to either domain or between the domains. Preferred fusion proteins comprise the amino acid sequence set forth in any one of SEQ ID NOs: 3-5.

In some embodiments, soluble ENPP1 polypeptides of the present disclosure contain one or more heterologous moieties. Optionally, a soluble ENPP1 polypeptide includes one or more heterologous moieties selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, a soluble ENPP1 polypeptide disclosed herein is further modified. Such modifications include, but are not limited to, acetylation, carboxylation, glycosylation, phosphorylation, lipidation, and acylation. As a result, the soluble ENPP1 polypeptide may contain non-amino acid elements, such as polyethylene glycols, lipids, polysaccharide or monosaccharide, and phosphates. Effects of such non-amino acid elements on the functionality of a soluble ENPP1 polypeptide may be tested as described herein for other soluble ENPP1 polypeptides. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the soluble ENPP1 polypeptides.

As used herein, percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. In some embodiments, alignment is performed using the CLUSTAL OMEGA software. Those skilled in the art can determine appropriate parameters for aligning sequences, including any algorithms needed to achieve maximal alignment over the full length of the sequences being compared.

### 6. Determining Solubility

In some embodiments, the activity of soluble ENPP1 polypeptides may also be tested in a cell-based or in vivo assay. For example, the effect of a soluble ENPP1 polypeptide on the production of inorganic pyrophosphates (PPi) can be measured. Specifically, the pyrophosphatase/phosphodiesterase domain of an ENPP1 protein hydrolyzes extracellular nucleotide triphosphates to produce inorganic pyrophosphates (PPi) and is generally soluble. This activity can be measured using a pNP-TMP assay as well as an HPLC-based ATP hydrolysis assay, as previously described (Saunders et al., 2008, Mol. Cancer Ther. 7(10):3352-62; Albright et al., 2015, Nat Comm. 6:10006). The effect of soluble ENPP1 polypeptides on the expression of genes involved in ENPP1 associated diseases such as ARHR2 (e.g., transcription of fibroblast growth factor 23 in osteoblasts and osteoclasts) can be assessed. This may as needed be performed in the presence of one or more nucleotide triphosphates or other ENPP1 substrates, and cells may be transfected so as to produce a soluble ENPP1 polypeptide. Likewise, a soluble ENPP1 polypeptide may be administered to a mouse or other animal and effects on ENPP1 associated diseases may be assessed using art-recognized methods.

In some embodiments, ENPP1 polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) analyses. Methods for assessment of purity are well known in the art [see, e.g., Flatman et al., 2007, J. Chromatogr. B 848:79-87]. In some embodiments, soluble ENPP1 polypeptides to be used in accordance with the methods described herein are recombinant polypeptides.

### 7. ENPP1 Production

ENPP1 polypeptides of the disclosure can be produced by a variety of art-known techniques. For example, polypeptides of the disclosure can be synthesized using standard protein chemistry techniques such as those described in Bodansky, Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus, Yeast Pichia] as is well known in the art. The protein can be produced in either adherent or suspension cells. In some embodiments, the fusion protein is expressed in CHO cells. To establish stable cell lines the nucleic acid sequence encoding ENPP1 constructs are cloned into an appropriate vector for large scale protein production. In certain embodiments, the modified or unmodified polypeptides of the disclosure may be produced by digestion of recombinantly produced full-length ENPP1 polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ENPP1 polypeptides using chemical cleavage (e.g., cyanogen bromide, hydroxylamine, and so forth).

### 8. Expression Systems

Many expression systems are known and can be used for the production of ENPP1 fusion protein, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired protein can be produced in conventional ways, for example from a coding sequence inserted in the host chromosome or on a free plasmid.

The yeasts can be transformed with a coding sequence for the desired protein in any of the usual ways (e.g., electroporation). Methods for transformation of yeast by electroporation are disclosed in Becker & Guarente, 1990, Methods Enzymol. 194: 182. Successfully transformed cells, i.e., cells that contain a DNA construct of the present disclosure, can be identified by well-known techniques. For example, cells resulting from the introduction of an expression construct can be grown to produce an ENPP1 polypeptide. Cells can be harvested and lysed and their DNA content examined for the presence of the DNA using a method, such as that described by Southern, 1975, J. Mol. Biol, 98:503 and/or Berent et al., 1985, Biotech 3:208. Alternatively, the presence of the protein in the supernatant can be detected using antibodies.

Useful yeast plasmid vectors include pRS403-406 and pRS413-416 and are generally available front Stratagene Cloning Systems, La Jolla, CA, USA Plasmids pRS403, pRS404, pRS405 and pRS406 are Yeast Integrating plasmids (Yips) and incorporate the yeast selectable markers I-11S3, TRP1, LEU2 and 1JRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. As a result, the products of the reaction are DNA segments carrying polymeric linker sequences at their ends. These DNA segments can be cleaved with an appropriate restriction enzyme and ligated to an expression vector that has been cleaved with an enzyme that produces termini compatible with those of the DNA segment.

Clones of single, stably transfected cells are then established and screened for high expressing clones of the desired ENPP1 fusion protein. Screening of the single cell clones for ENPP1 protein expression can be accomplished in a high-throughput manner in 96 well plates using the synthetic enzymatic substrate pNP-TMP as previously described (Albright et al., 2015, Nat. Commun. 6:10006). Upon identification of high expressing clones through screening, protein production can be accomplished in shaking flasks or bio-reactors are previously described in Albright et al., 2015, Nat. Commun. 6:10006.

### 9. ENPP1 Purification

Purification of ENPP1 can be accomplished using a combination of standard purification techniques known in the art. Following purification, ENPP1-Fc can be dialyzed into PBS supplemented with Zn²⁺ and Mg²⁺ (PBSplus) concentrated to between 5 and 7 mg/ml, and frozen at −80° C. in aliquots of 200-500 pl. Aliquots can be thawed immediately prior to use and the specific activity of the solution can be adjusted to 31.25 au/ml (or about 0.7 mg/ml depending on the preparation) by dilution in PBSplus.

### 10. Route and Frequency of Administration

The polypeptide may be administered acutely or chronically to the subject. In certain embodiments, a second dosage of a soluble ENPP1 polypeptide or ENPP1 fusion polypeptide disclosed herein is administered after a suitable time interval of about after two days, after four days, after a week, or after a month to the subject or even less frequently, such as once every several months or even once a year or less. The frequency of the dose is readily apparent to the skilled artisan and depends upon any number of factors, such as, but not limited to, the type and severity of the disease being treated, and the type and age of the patient.

A dose amount or frequency may be selected so that the steady state level of plasma PPi is maintained at a constant or steady state level, and/or so as to achieve a continuous level of plasma PPi that is either close to the normal (2-3 μM) level or above (30-50% higher than) normal levels of PPi and does not return to the lower level of PPi that the subject had prior to the administration of first dosage of constructs disclosed herein.

Alternative, the ENPP1 agent may be administered at appropriate time intervals of either every 2 days, or every 4 days, every week or every month so as to achieve a constant level of enzymatic activity of ENPP1.

Alternatively, an ENPP1 agent according to the disclosure is administered at an appropriate time interval of every 2 days, or every 4 days, or every week or every month by monitoring one or more symptoms of a subject's disease or disorder.

Without wishing to be bound by theory, it is believed that maintaining a steady state concentration of plasma PPi at normal levels reduces and/or prevents progression of pathological calcification of subjects.

In certain embodiments, the polypeptide is administered locally, regionally, parenterally or systemically to the subject. In some embodiments, the polypeptide is administered subcutaneously.

As used herein, “parenteral administration” of a formulation includes any route of administration characterized by physical breaching of a tissue of a subject and administration of the ENPP1 agent through the breach in the tissue. Parenteral administration thus includes, but is not limited to, administration of an ENPP1 agent by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

The regimen of administration may affect what constitutes an effective amount. For example, several divided dosages, as well as staggered dosages may be administered in a given time period (daily) or sequentially, or the dose may be continuously infused, or may be a bolus injection. Further, selection of a recited dose of an ENPP1 agent may be indicated by the exigencies of the therapeutic or prophylactic situation.

Administration of the compositions of the present disclosure (e.g., soluble ENPP1 polypeptides and fusion proteins thereof) to a patient, such as a mammal (i.e., a human), may be carried out using known procedures, at dosages and for periods of time effective to treat a disease or disorder in the patient. An effective amount of the recited dosages of an ENPP1 agent necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. A selected dosage is determined based on the biological activity of the therapeutic compound which in turn depends on the half-life and the area under the plasma time of the therapeutic compound curve.

### 11. Prophylactic Administration

Armed with the disclosure herein, one skilled in the art would thus appreciate that the prevention of a disease or disorder in a subject encompasses administering to a subject an ENPP1 polypeptide as a preventative measure against the disease or disorder.

The relative amounts of the active ingredient (e.g., soluble ENPP1 polypeptides and fusion proteins thereof), the pharmaceutically acceptable carrier, and any additional ingredients in a formulation disclosed herein will vary, depending upon the identity, size, and condition of the subject treated and further depending upon the route by which the composition is to be administered. By way of example, the composition may comprise between about 0.1% and about 100% (w/w) active ingredient.

### 12. ENPP1 Polypeptide Sequences

TABLE 1
Sequences

| SEQ | | |
|---|---|---|
| ID | | Descrip- |
| NO | Sequence | tion |
| 1 | 1 MERDGCAGGG SRGGEGGRAP REGPAGNGRD RGRSHAAEAP GDPQAAASLL | Full, un- |
| | 51 APMDVGEEPL EKAARARTAK DPNTYKVLSL VLSVCVLTTI LGCIFGLKPS | processed |
| | 101 CAKEVKSCKG RCFERTFGNC RCDAACVELG NCCLDYQETC IEPEHIWTCN | amino |
| | 151 KFRCGEKRLT RSLCACSDDC KDKGDCCINY SSVCQGEKSW VEEPCESINE | acid |
| | 201 PQCPAGFETP PTLLFSLDGF RAEYLHTWGG LLPVISKLKK CGTYTKNMRP | sequence |
| | 251 VYPTKTFPNH YSIVTGLYPE SHGIIDNKMY DPKMNASFSL KSKEKFNPEW | of |
| | 301 YKGEPIWVTA KYQGLKSGTF FWPGSDVEIN GIFPDIYKMY NGSVPFEERI | wild-type |
| | 351 LAVLQWLQLP KDERPHFYTL YLEEPDSSGH SYGPVSSEVI KALQRVDGMV | ENPP1 |
| | 401 GMLMDGLKEL NLHRCLNLIL ISDHGMEQGS CKKYIYLNKY LGDVKNIKVI | precursor |
| | 451 YGPAARLRPS DVPDKYYSFN YEGIARNLSC REPNQHFKPY LKHFLPKRLH | protein |
| | 501 FAKSDRIEPL TFYLDPQWQL ALNPSERKYC GSGFHGSDNV FSNMQALFVG | |
| | 551 YGPGFKHGIE ADTFENIEVY NLMCDLLNLT PAPNNGTHGS LNHLLKNPVY | |
| | 601 TPKHPKEVHP LVQCPFTRNP RDNLGCSCNP SILPIEDFQT QFNLTVAEEK | |
| | 651 IIKHETLPYG RPRVLQKENT ICLLSQHQFM SGYSQDILMP LWTSYTVDRN | |
| | 701 DSFSTEDFSN CLYQDFRIPL SPVHKCSFYK NNTKVSYGFL SPPQLNKNSS | |
| | 751 GIYSEALLTT NIVPMYQSFQ VIWRYFHDTL LRKYAEERNG VNVVSGPVFD | |
| | 801 FDYDGRCDSL ENLRQKRRVI RNQEILIPTH FFIVLTSCKD TSQTPLHCEN | |
| | 851 LDTLAFILPH RTDNSESCVH GKHDSSWVEE LLMLHRARIT DVEHITGLSF | |
| | 901 YQQRKEPVSD ILKLKTHLPT FSQED | |
| 2 | 1 PSCAKEVKSC KGRCFERTFG NCRCDAACVE LGNCCLDYQE TCIEPEHIWT | The pro- |
| | 51 CNKFRCGEKR LTRSLCACSD DCKDKGDCCI NYSSVCQGEK SWVEEPCESI | cessed |
| | 101 NEPQCPAGFE TPPTLLFSLD GFRAEYLHTW GGLLPVISKL KKCGTYTKNM | (mature) |
| | 151 RPVYPTKTFP NHYSIVTGLY PESHGIIDNK MYDPKMNASF SLKSKEKFNP | extra- |
| | 201 EWYKGEPIWV TAKYQGLKSG TFFWPGSDVE INGIFPDIYK MYNGSVPFEE | cellular |
| | 251 RILAVLQWLQ LPKDERPHFY TLYLEEPDSS GHSYGPVSSE VIKALQRVDG | ENPP1 |
| | 301 MVGMLMDGLK ELNLHRCLNL ILISDHGMEQ GSCKKYIYLN KYLGDVKNIK | polypep- |
| | 351 VIYGPAARLR PSDVPDKYYS FNYEGIARNL SCREPNQHFK PYLKHFLPKR | tide |
| | 401 LHFAKSDRIE PLTFYLDPQW QLALNPSERK YCGSGFHGSD NVFSNMQALF | sequence |
| | 451 VGYGPGFKHG IEADTFENIE VYNLMCDLLN LTPAPNNGTH GSLNHLLKNP | |
| | 501 VYTPKHPKEV HPLVQCPFTR NPRDNLGCSC NPSILPIEDF QTQFNLTVAE | |
| | 551 EKIIKHETLP YGRPRVLQKE NTICLLSQHQ FMSGYSQDIL MPLWTSYTVD | |
| | 601 RNDSFSTEDF SNCLYQDFRI PLSPVHKCSF YKNNTKVSYG FLSPPQLNKN | |
| | 651 SSGIYSEALL TTNIVPMYQS FQVIWRYFHD TLLRKYAEER NGVNVVSGPV | |
| | 701 FDFDYDGRCD SLENLRQKRR VIRNQEILIP THFFIVLTSC KDTSQTPLHC | |
| | 751 ENLDTLAFIL PHRTDNSESC VHGKHDSSWV EELLMLHRAR ITDVEHITGL | |
| | 801 SFYQQRKEPV SDILKLKTHL PTFSQED | |
| 3 | FTAGLKPSCAKEVKSCKGRCFERTFGNCRCDAACVELGNCCLDYQETCIEPEHIWTCNKFRC | ENPP1- |
| | GEKRLTRSLCACSDDCKDKGDCCINYSSVCQGEKSWVEEPCESINEPQCPAGFETPPTLLFSL | linker- |
| | DGFRAEYLHTWGGLLPVISKLKKCGTYTKNMRPVYPTKTFPNHYSIVTGLYPESHGIIDNKM | hIgG1 Fc |
| | YDPKMNASFSLKSKEKFNPEWYKGEPIWVTAKYQGLKSGTFFWPGSDVEINGIFPDIYKMYN | construct |
| | GSVPFEERILAVLQWLQLPKDERPHFYTLYLEEPDSSGHSYGPVSSEVIKALQRVDGMVGML | |
| | MDGLKELNLHRCLNLILISDHGMEQGSCKKYIYLNKYLGDVKNIKVIYGPAARLRPSDVPDK | |
| | YYSFNYEGIARNLSCREPNQHFKPYLKHFLPKRLHFAKSDRIEPLTFYLDPQWQLALNPSERK | |
| | YCGSGFHGSDNVFSNMQALFVGYGPGFKHGIEADTFENIEVYNLMCDLLNLTPAPNNGTHG | |
| | SLNHLLKNPVYTPKHPKEVHPLVQCPFTRNPRDNLGCSCNPSILPIEDFQTQFNLTVAEEKIIK | |
| | HETLPYGRPRVLQKENTICLLSQHQFMSGYSQDILMPLWTSYTVDRNDSFSTEDFSNCLYQD | |
| | FRIPLSPVHKCSFYKNNTKVSYGFLSPPQLNKNSSGIYSEALLTTNIVPMYQSFQVIWRYFHDT | |
| | LLRKYAEERNGVNVVSGPVFDFDYDGRCDSLENLRQKRRVIRNQEILIPTHFFIVLTSCKDTS | |
| | QTPLHCENLDTLAFILPHRTDNSESCVHGKHDSSWVEELLMLHRARITDVEHITGLSFYQQR | |
| | KEPVSDILKLKTHLPTFSQEDLINDKTHTCPPCPAPELLGGPSVFLFPPKPKDTLMISRTPE | |
| | VTCVVVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVFSCSVMHEAL | |
| | HNHYTQKSLSLSPGK | |
| 4 | GLKPSCAKEVKSCKGRCFERTFGNCRCDAACVELGNCCLDYQETCIEPEHIWTCNKFRCGEK | ENPP1- |
| | RLTRSLCACSDDCKDKGDCCINYSSVCQGEKSWVEEPCESINEPQCPAGFETPPTLLFSLDGF | linker- |
| | RAEYLHTWGGLLPVISKLKKCGTYTKNMRPVYPTKTFPNHYSIVTGLYPESHGIIDNKMYDP | hIgG1 Fc |
| | KMNASFSLKSKEKFNPEWYKGEPIWVTAKYQGLKSGTFFWPGSDVEINGIFPDIYKMYNGSV | construct |
| | PFEERILAVLQWLQLPKDERPHFYTLYLEEPDSSGHSYGPVSSEVIKALQRVDGMVGMLMD | |
| | GLKELNLHRCLNLILISDHGMEQGSCKKYIYLNKYLGDVKNIKVIYGPAARLRPSDVPDKYY | |
| | SFNYEGIARNLSCREPNQHFKPYLKHFLPKRLHFAKSDRIEPLTFYLDPQWQLALNPSERKYC | |
| | GSGFHGSDNVFSNMQALFVGYGPGFKHGIEADTFENIEVYNLMCDLLNLTPAPNNGTHGSL | |
| | NHLLKNPVYTPKHPKEVHPLVQCPFTRNPRDNLGCSCNPSILPIEDFQTQFNLTVAEEKIIKHE | |
| | TLPYGRPRVLQKENTICLLSQHQFMSGYSQDILMPLWTSYTVDRNDSFSTEDFSNCLYQDFRI | |
| | PLSPVHKCSFYKNNTKVSYGFLSPPQLNKNSSGIYSEALLTTNIVPMYQSFQVIWRYFHDTLL | |
| | RKYAEERNGVNVVSGPVFDFDYDGRCDSLENLRQKRRVIRNQEILIPTHFFIVLTSCKDTSQT | |
| | PLHCENLDTLAFILPHRTDNSESCVHGKHDSSWVEELLMLHRARITDVEHITGLSFYQQRKEP | |
| | VSDILKLKTHLPTFSQEDLINDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTCLVKGFYPSDI | |
| | AVEWESNGQPENNYKTTPPVLDSDGSFFLYSKLTVDKSRWQQGNVFSCSVMHEALHN | |
| | HYTQKSLSLSPGK | |
| 5 | PSCAKEVKSCKGRCFERTFGNCRCDAACVELGNCCLDYQETCIEPEHIWTCNKFRCGEKRLT | ENPP1- |
| | RSLCACSDDCKDKGDCCINYSSVCQGEKSWVEEPCESINEPQCPAGFETPPTLLESLDGFRAE | linker- |
| | YLHTWGGLLPVISKLKKCGTYTKNMRPVYPTKTFPNHYSIVTGLYPESHGIIDNKMYDPKMN | hIgG1 Fc |
| | ASFSLKSKEKFNPEWYKGEPIWVTAKYQGLKSGTFFWPGSDVEINGIFPDIYKMYNGSVPFEE | construct |
| | RILAVLQWLQLPKDERPHFYTLYLEEPDSSGHSYGPVSSEVIKALQRVDGMVGMLMDGLKE | |
| | LNLHRCLNLILISDHGMEQGSCKKYIYLNKYLGDVKNIKVIYGPAARLRPSDVPDKYYSFNY | |
| | EGIARNLSCREPNQHFKPYLKHFLPKRLHFAKSDRIEPLTFYLDPQWQLALNPSERKYCGSGF | |
| | HGSDNVFSNMQALFVGYGPGFKHGIEADTFENIEVYNLMCDLLNLTPAPNNGTHGSLNHLL | |
| | KNPVYTPKHPKEVHPLVQCPFTRNPRDNLGCSCNPSILPIEDFQTQFNLTVAEEKIIKHETLPY | |
| | GRPRVLQKENTICLLSQHQFMSGYSQDILMPLWTSYTVDRNDSFSTEDFSNCLYQDFRIPLSP | |
| | VHKCSFYKNNTKVSYGFLSPPQLNKNSSGIYSEALLTTNIVPMYQSFQVIWRYFHDTLLRKY | |
| | AEERNGVNVVSGPVFDFDYDGRCDSLENLRQKRRVIRNQEILIPTHFFIVLTSCKDTSQTPLH | |
| | CENLDTLAFILPHRTDNSESCVHGKHDSSWVEELLMLHRARITDVEHITGLSFYQQRKEPVSD | |
| | ILKLKTHLPTFSQEDLINDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLNGKEYK | |
| | CKVSNKALPAPIEKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSVMHEALHNHYT | |
| | QKSLSLSPGK | |
| 6 | DKTHTCPPCPAPELLGGPSVFLFPPKPKDTLMISRTPEVTCVVVDVSHEDPEVKFNWYVDGV | Human |
| | EVHNAKTKPREEQYNSTYRVVSVLTVLHQDWLNGKEYKCKVSNKALPAPIEKTISKAKGQP | IgG1 Fc |
| | REPQVYTLPPSREEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 118 | X-PSCAKEVKSCKGRCFERTFGNCRCDAACVELGNCCLDYQETCIEPEHIWTCNKFRCGEKR | BL-1118 |
| | LTRSLCACSDDCKDKGDCCINYSSVCQGEKSWVEEPCESINEPQCPAGFETPPTLLESLD | (long |
| | GFRAEYLHTWGGLLPVISKLKKCGTYTKNMRPVYPTKTFPNHYSIVTGLYPESHGIIDNK | acting) |
| | MYDPKMNASFSLKSKEKFNPEWYKGEPIWVTAKYQGLKSGTFFWPGSDVEINGTFPDIYK | |
| | MYNGSVPFEERILAVLQWLQLPKDERPHFYTLYLEEPDSSGHSYGPVSSEVIKALQRVDG | |
| | MVGMLMDGLKELNLHRCLNLILISDHGMEQGSCKKYIYLNKYLGDVKNIKVIYGPAARLR | |
| | PSDVPDKYYSFNYEGIARNLSCREPNQHFKPYLKHFLPKRLHFAKSDRIEPLTFYLDPQW | |
| | QLALNPSERKYCGSGFHGSDNVFSNMQALFVGYGPGFKHGIEADTFENIEVYNLMCDLLN | |
| | LTPAPNNGTHGSLNHLLKNPVYTPKHPKEVHPLVQCPFTRNPRDNLGCSCNPSILPIEDF | |
| | QTQFNLTVAEEKIIKHETLPYGRPRVLQKENTICLLSQHQFMSGYSQDILMPLWTSYTVD | |
| | RNDSFSTEDFSNCLYQDFRIPLSPVHKCSFYKNNTKVSYGFLSPPQLNKNSSGIYSEALL | |
| | TTNIVPMYQSFQVIWRYFHDTLLRKYAEERNGVNVVSGPVFDFDYDGRCDSLENLRQKRR | |
| | VIRNQEILIPTHFFIVLTSCKDTSQTPLHCENLDTLAFILPHRTDNSESCVHGKHDSSWV | |
| | EELLMLHRARITDVEHITGLSFYQQRKEPVSDILKLKTHLPTFSQEDRSDKTHTCPPCPA | |
| | PELLGGPSVFLFPPKPKDTLYITREPEVTCVVVDVSHEDPEVKFNWYVDGVEVHNAKTK | |
| | P | |
| | REEQYNSTYRVVSVLTVLHQDWLNGKEYKCKVSNKALPAPIEKTISKAKGQPREPQVY | |
| | TL | |
| | PPSREEMTKNQVSLTCLVKGFYPSDIAVEWESNGQPENNYKTTPPVLDSDGSFFLYSKL | |
| | T | |
| | VDKSRWQQGNVFSCSVMHEALHNHYTQKSLSLSPGK-Y | |
| 119 | X-PSCAKEVKSCKGRCFERTFGNCRCDAACVELGNCCLDYQETCIEPEHIWTCNKFRCGEKR | BL-782 |
| | LTRSLCACSDDCKDKGDCCINYSSVCQGEKSWVEEPCESINEPQCPAGFETPPTLLESLD | |
| | GFRAEYLHTWGGLLPVISKLKKCGTYTKNMRPVYPTKTFPNHYSIVTGLYPESHGIIDNK | |
| | MYDPKMNASFSLKSKEKFNPEWYKGEPIWVTAKYQGLKSGTFFWPGSDVEINGIFPDIYK | |
| | MYNGSVPFEERILAVLQWLQLPKDERPHFYTLYLEEPDSSGHSYGPVSSEVIKALQRVDG | |
| | MVGMLMDGLKELNLHRCLNLILISDHGMEQGSCKKYIYLNKYLGDVKNIKVIYGPAARLR | |
| | PSDVPDKYYSFNYEGIARNLSCREPNQHFKPYLKHFLPKRLHFAKSDRIEPLTFYLDPQW | |
| | QLALNPSERKYCGSGFHGSDNVFSNMQALFVGYGPGFKHGIEADTFENIEVYNLMCDLLN | |
| | LTPAPNNGTHGSLNHLLKNPVYTPKHPKEVHPLVQCPFTRNPRDNLGCSCNPSILPIEDF | |
| | QTQFNLTVAEEKIIKHETLPYGRPRVLQKENTICLLSQHQFMSGYSQDILMPLWTSYTVD | |
| | RNDSFSTEDFSNCLYQDFRIPLSPVHKCSFYKNNTKVSYGFLSPPQLNKNSSGIYSEALL | |
| | TTNIVPMYQSFQVIWRYFHDTLLRKYAEERNGVNVVSGPVFDFDYDGRCDSLENLRQKRR | |
| | VIRNQEILIPTHFFIVLTSCKDTSQTPLHCENLDTLAFILPHRTDNSESCVHGKHDSSWV | |
| | EELLMLHRARITDVEHITGLSFYQQRKEPVSDILKLKTHLPTFSQEDRSDKTHTCPPCPA | |
| | PELLGGPSVFLFPPKPKDTLMISRTPEVTCVVVDVSHEDPEVKFNWYVDGVEVHNAKT | |
| | KP | |
| | REEQYNSTYRVVSVLTVLHQDWLNGKEYKCKVSNKALPAPIEKTISKAKGQPREPQVY | |
| | TL | |
| | PPSREEMTKNQVSLTCLVKGFYPSDIAVEWESNGQPENNYKTTPPVLDSDGSFFLYSKL | |
| | T | |
| | VDKSRWQQGNVFSCSVMHEALHNHYTQKSLSLSPGK-Y | |

For SEQ ID NO: 118 and 119:
X=export sequences
Y=Bone targeting tag or a stop codon (i.e. no amino acid as marked by * Non-limiting examples of bone targeting sequences include, but are not limited to, DDDDDDDD and/or ELEELEEL.
Non-bold, non-underlined font=linker region, which can be various lengths and so forth. Amino acid composition of the linker can be variable. In certain non-limiting embodiments, the linker region can include residues such as histidine to discourage non-specific protease cleavage (i.e., HS or HG instead of RS or RG).

```
Bold font = Human IgG1 (optimized in BL-1118 with the M883Y
S885T T887E mutations highlighted in bold italics)
Underlined font = extracellular domain of human ENPP1
(optimized in BL-1118 with the
I256T substitution, highlighted in bold underlined)
SEQ ID NO: 7 (Mouse ENPP1- NCBI accession NP_001295256.1)
    1 MERDGDQAGH GPRHGSAGNG RELESPAAAS LLAPMDLGEE PLEKAERARP AKDPNTYKVL
  61 SLVLSVCVLT TILGCIFGLK PSCAKEVKSC KGRCFERTFS NCRCDAACVS LGNCCLDFQE
121 TCVEPTHIWT CNKFRCGEKR LSRFVCSCAD DCKTHNDCCI NYSSVCQDKK SWVEETCESI
181 DTPECPAEFE SPPTLLFSLD GFRAEYLHTW GGLLPVISKL KNCGTYTKNM RPMYPTKTFP
241 NHYSIVTGLY PESHGIIDNK MYDPKMNASF SLKSKEKFNP LWYKGQPIWV TANHQEVKSG
301 TYFWPGSDVE IDGILPDIYK VYNGSVPFEE RILAVLEWLQ LPSHERPHFY TLYLEEPDSS
361 GHSHGPVSSE VIKALQKVDR LVGMLMDGLK DLGLDKCLNL ILISDHGMEQ GSCKKYVYLN
421 KYLGDVNNVK VVYGPAARLR PTDVPETYYS FNYEALAKNL SCREPNQHFR PYLKPFLPKR
481 LHFAKSDRIE PLTFYLDPQW QLALNPSERK YCGSGFHGSD NLFSNMQALF IGYGPAFKHG
541 AEVDSFENIE VYNLMCDLLG LIPAPNNGSH GSLNHLLKKP IYNPSHPKEE GFLSQCPIKS
601 TSNDLGCTCD PWIVPIKDFE KQLNLTTEDV DDIYHMTVPY GRPRILLKQH RVCLLQQQQF
661 LTGYSLDLLM PLWASYTFLS NDQFSRDDFS NCLYQDLRIP LSPVHKCSYY KSNSKLSYGF
721 LTPPRLNRVS NHIYSEALLT SNIVPMYQSF QVIWHYLHDT LLQRYAHERN GINVVSGPVF
781 DFDYDGRYDS LEILKQNSRV IRSQEILIPT HFFIVLTSCK QLSETPLECS ALESSAYILP
841 HRPDNIESCT HGKRESSWVE ELLTLHRARV TDVELITGLS FYQDRQESVS ELLRLKTHLP
901 IFSQED
SEQ ID NO: 8 (Cow ENPP1- NCBI accession NP_001193141.1)
    1 MERDSCAGGG SRGGEGGRGP REGLAGNGRD PGPGRAAEAS GEPQAAASLL APMDLGEEPL
  61 ERAARARPAK DPNTYKVLSL VLSVCVLTTI LGCIFGLKPS CAKEIKSCKG RCFERTFGNC
121 RCDAACVDLG NCCLDYQETC IEPERIWTCT KFRCGEKRLS RSLCSCSDDC KDKGDCCINH
181 GSVCRGEKSW AEEECDSIDE PQCPAGFETP PTLLFSLDGF RAEYLHTWGG LLPVISKLKT
241 CGTYTKNMRP VYPTKTFPNH YSIVTGLYPE SHGIIDNNIY DPQMNANFAL KNKEKFNPEW
301 YKGEPIWLTA KYQGLKTGTF FWPGSDVKIN GIFPDIYKIY NVSVPFEERI LAILKWLQLP
361 KDERPHFYTL YLEEPDSSGH SYGPVSSEVI RALQRVDNMV GMLMDGLKEL NLHRCLNLIL
421 ISDHGMEQGS CKKYVYLNKY LGDTKDYKVV YGPAARLRPS DVPDKYYSFD YEGIAKNLSC
481 QEPNQHFKPY LKHFLPKRLH FAKNDRIERL TFYLDPQWQL ALNPSERKYC GGGFHGSDNT
541 FLNMQALFIG YGPGFKHSTE VDSFENIEVY NLMCDLLNLT PAPNNGTHGS LNHLLSNPVY
601 TPKHPKEVRP LVQCPFTRAP RESLDCSCDP SILPIVDFQT QLNLTMAEEK TIKRGALPYG
661 RPRVLQNSTV CLLYQHQFVS GYSRDILMPL WTSYTIGRND SFSTEDFSNC LYQDLRIPLS
721 PVHKCSFYKN NAKLSYGLLS PPQLHKGSSQ VYSEALLTTN IVPMYQSFQV IWHYLHGTLL
781 QRYAEERNGL NVVSGPVFDS DYDGRYDSLE TLKQNSKIIR NLEVLIPTHF FLVLTSCKNT
841 SQTPLQCENL DAMAFILPHK TDNSESCAHG KHESLWVEEL LKLHTARITD VEHITGLSFY
901 QERKEPISDI LKLKTHLPTF NQED
SEQ ID NO: 9 (Rabbit ENPP1- NCBI accession NP_001162404.1)
    1 MERDGCAGGG SRGGEGGRAP REGPAGNSRD PGRSHAAEAP GNPQAAASLL APMDVGEEPL
  61 EKAARARTAK DPNTYKVLSL VLSVCVLTTI LGCIFGLKPS CAKEVKSCKG RCFERTFGNC
121 RCDAACVELG NCCLDYQETC IEPEHIWTCN KFRCGEKRLT RSLCACSDDC KDQGDCCINY
181 SSVCQGEKSW VEEPCESINE PQCPAGFETP PTLLFSLDGF RAEYLHTWGG LLPVISKLKK
241 CGTYTKNMRP VYPTKTFPNH YSIVTGLYPE SHGIIDNKMY DPKMNASFSL KSKEKENPEW
301 YKGEPIWVTA KYQGLKSGTF FWPGSDVEIN GIFPDIYKMY NGSVPFEERI LAVLQWLQLP
361 KDERPHFYTL YLEEPDSSGH SYGPVSSEVI KALQRVDNMV GMLMDGLKEL NLHRCLNLIL
421 VSDHGMEQGS CKKYIYLNKY LGDVKNIKVI YGPAARLRPS DVPDKYYSEN YEGIARNLSC
481 REPNQHFKPY LKHFLPKRLH FAKSDRIEPL TFYLDPQWQL ALNPSERKYC GSGFHGSDNI
541 FSNMQALFVG YGPGFKHGIE VDTFENIEVY NLMCDLLNLT PAPNNGTHGS LNHLLKNPVY
601 TPKHPKEVHP LIQCPFTRNP RDNLGCSCNP SILPIEDFQT QFNLTVAEEK NIKHETLPYG
661 RPRVLQKKNT ICLLSQHQFM SGYSQDILMP LWTSYTVDRN DSFSTEDFSN CLYQDFRISL
721 SPVHKCSFYK NNTKVSYGFL SPPQLNKNSR GIYSEALLTT NIVPMYQSFQ VIWRYFHDTL
781 LRKYAEERNG VNVVSGPVFD FDYDGRYDSL EILRQKRRVI RNQEILIPTH FFIVLTSCKD
841 ASQTPLHCEN LDTLAFILPH RTDNSESCLH GKHESSWVEE LLMLHRARIT DVEHITGLSF
901 YQQRKEPVSD ILKLKTHLPT FSQED
SEQ ID NO: 10 (Baboon ENPPI- NCBI accession NP_001076211.2)
    1 MERDGCAGGG SQGGGKGGRG PREGLAGNGR DPSHGQASEA PGDPQAAASL LAPMDLGEEP
  61 LEKAAGARPA KDPNTYKVLS LVLSVCVLTT ILGCIFGLKP SCAKEVKSCK GRCFERTFGN
121 CRCDVACVDL GNCCLDYQET CIEPERIWTC NKFRCGEKRL SRSLCACSDD CKERGDCCIN
181 YSAVCQGEKS WVEETCENIN EPQCPEGFEM PPTLLESLDG FRAEYLHTWG GLLPVISKLK
241 KCGTYAKNMR PVYPTKTFPN HYSIVTGLYP ESHGIIDNKM YDPKMNASFS LKSKEKFNPE
301 WYKGEPIWLT AKYQGLRSGT FFWPGSDVKI NGIFPDIYKI YNGSVPFEER ILAILKWLRL
361 PKDERPHFYT LYLEEPDSSG HSYGPVSSEV IKALQRVDNM VGMLMDGLKE LNLHQCLNLI
421 LISDHGMEQG SCKKYIYLNK YLGDTKNIKV IYGPAARLRP SDVPEKYYSF NYENIARNLS
481 CREPNQHFKP YLKHFLPKRL HFAKSDRIEP LTFYLDPQWQ LALSPSERKY CGSGFHGSDN
541 VFSNMQALFV GYGPGFQHGI EVDSFENIEV YNLMCDLLNL TPAPNNGTHG SLNHLLKNPI
601 YTPKHPKEVQ PSVQCPLAGS PRDSLGCSCN PSILPIVDFQ TQFNLTTAEE KNINRASLPY
661 GRPRLLQKKS SVCLLYQHQF VSGYSHDVLM PLWTSYTVNR NDSFSTEDFS NCLYQDLRIS
721 FSPIHNCSFY KNNAKLSYGF LSPPQLSKDS SQIYSEALLT SNIVPMYQSF QVIWRYFHDT
781 LLQRYAEERN SINVVSGPVF DSDYDGRYDS SEALKRNRRV IRNQEILIPT HFFIVITSCK
841 NTSQTPLQCD NLDPLAFILP HRSDNSESCV HEKRESSWIE ELLMMHRARI MDVEHITGLS
901 FYQERKEPVS DILKLKTHLP TVSQED
SEQ ID NO: 118 (Construct 1118 - Soluble ENPP1-Fc)
MRGPAVLLTVALATLLAPGAGAPSCAKEVKSCKGRCFERTFGNCRCDAACVELGNCCLDYQETCIEPE
HIWTCNKFRCGEKRLTRSLCACSDDCKDKGDCCINYSSVCQGEKSWVEEPCESINEPQCPAGFETPPTL
LFSLDGFRAEYLHTWGGLLPVISKLKKCGTYTKNMRPVYPTKTFPNHYSIVTGLYPESHGIIDNKMYDP
KMNASFSLKSKEKFNPEWYKGEPIWVTAKYQGLKSGTFFWPGSDVEINGTFPDIYKMYNGSVPFEERIL
AVLQWLQLPKDERPHFYTLYLEEPDSSGHSYGPVSSEVIKALQRVDGMVGMLMDGLKELNLHRCLNL
ILISDHGMEQGSCKKYIYLNKYLGDVKNIKVIYGPAARLRPSDVPDKYYSFNYEGIARNLSCREPNQHF
KPYLKHFLPKRLHFAKSDRIEPLTFYLDPQWQLALNPSERKYCGSGFHGSDNVFSNMQALFVGYGPGF
KHGIEADTFENIEVYNLMCDLLNLTPAPNNGTHGSLNHLLKNPVYTPKHPKEVHPLVQCPFTRNPRDN
LGCSCNPSILPIEDFQTQFNLTVAEEKIIKHETLPYGRPRVLQKENTICLLSQHQFMSGYSQDILMPLWTS
YTVDRNDSFSTEDFSNCLYQDFRIPLSPVHKCSFYKNNTKVSYGFLSPPQLNKNSSGIYSEALLTTNIVP
MYQSFQVIWRYFHDTLLRKYAEERNGVNVVSGPVFDFDYDGRCDSLENLRQKRRVIRNQEILIPTHFFI
VLTSCKDTSQTPLHCENLDTLAFILPHRTDNSESCVHGKHDSSWVEELLMLHRARITDVEHITGLSFYQ
QRKEPVSDILKLKTHLPTFSQEDRSDKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
SEQ ID NO: 119 (Construct 2000 - Bone Targeted ENPP1-Fc)
MRGPAVLLTVALATLLAPGAGAPSCAKEVKSCKGRCFERTFGNCRCDAACVELGNCCLDYQETCIEPE
HIWTCNKFRCGEKRLTRSLCACSDDCKDKGDCCINYSSVCQGEKSWVEEPCESINEPQCPAGFETPPTL
LFSLDGFRAEYLHTWGGLLPVISKLKKCGTYTKNMRPVYPTKTFPNHYSIVTGLYPESHGIIDNKMYDP
KMNASFSLKSKEKFNPEWYKGEPIWVTAKYQGLKSGTFFWPGSDVEINGTFPDIYKMYNGSVPFEERIL
AVLQWLQLPKDERPHFYTLYLEEPDSSGHSYGPVSSEVIKALQRVDGMVGMLMDGLKELNLHRCLNL
ILISDHGMEQGSCKKYIYLNKYLGDVKNIKVIYGPAARLRPSDVPDKYYSFNYEGIARNLSCREPNQHF
KPYLKHFLPKRLHFAKSDRIEPLTFYLDPQWQLALNPSERKYCGSGFHGSDNVFSNMQALFVGYGPGF
KHGIEADTFENIEVYNLMCDLLNLTPAPNNGTHGSLNHLLKNPVYTPKHPKEVHPLVQCPFTRNPRDN
LGCSCNPSILPIEDFQTQFNLTVAEEKIIKHETLPYGRPRVLQKENTICLLSQHQFMSGYSQDILMPLWTS
YTVDRNDSFSTEDFSNCLYQDFRIPLSPVHKCSFYKNNTKVSYGFLSPPQLNKNSSGIYSEALLTTNIVP
MYQSFQVIWRYFHDTLLRKYAEERNGVNVVSGPVFDFDYDGRCDSLENLRQKRRVIRNQEILIPTHFFI
VLTSCKDTSQTPLHCENLDTLAFILPHRTDNSESCVHGKHDSSWVEELLMLHRARITDVEHITGLSFYQ
QRKEPVSDILKLKTHLPTFSQEDRSDKTHTCPPCPAPELLGGPSVFLFPPKPKDTLYITREPEVTCVVVDV
SHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGKDDDDDDDD*
```

### EXPERIMENTAL EXAMPLES

The disclosure is further described in detail by reference to the following experimental examples. These examples are provided for purposes of illustration only, and are not intended to be limiting unless so specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

The materials and methods employed in the following Examples are now described.

### Human Subjects

Patients' data, including laboratory data, X-rays, computed tomography (CT), and bone mineral density (BMD) was retrospectively collected and the skeletal and biochemical data in patients with ENPP1 mutations was reported.

### Mutational Analysis

Mutational analysis of genes associated with hereditary hypophosphatemic rickets, osteogenesis imperfecta (OI), and osteoporosis-pseudoglioma syndrome (OPPG) (Table 2) were performed, utilizing next-generation sequencing with the MiSeq Sequencing System at the Kazusa DNA Research Institute, as reported previously (Fujiki et al., 2018). Direct sequencing of the detected ENPP1 variants was performed in the family members of the probands as previously reported (Fujiki et al., 2018) or by using the primers listed in Table 2. The pathogenicity of the detected variants was assessed by the in silico tools PolyPhen-2, SIFT, and Mutation Taster (Adzhubei et al., 2010; Kumar et al., 2009; Schwarz et al., 2010).

TABLE 2
Genes tested in the present case series (A)
and primers used for targeted sequence of the
variant in ENPP1 gene (B)

| (A) Genes | |
|---|---|
| tested in | |
| the present | |
| report | Genes |
| Genes | BMP1, COL1A1, COL1A2, CRTAP, |
| associated | FKBP10, IFITM5, P3H1, PP1B, |
| with OI and | SERPINF1, SERPINH1, SP7, |
| OPPG | TMEM38B, WNT1, CREB3L1, |
| | SPARC, TENT5A, MBTPS2, MESD |
| Genes | PHEX, FGF23, DMP1, ENPP1, |
| associated | FAM20C, FGFR1, PTH1R, |
| with | SLC34A3, SLC9A3R1, CLCN5, |
| hypopho- | OCRL, CYP27B1, CYP2R1, VDR, |
| sphatemic | HNRNPC, CYP3A4, NF1, SLC34A1 |
| rickets | |

(B)Primers used for target sequence of the
variant in ENPPI gene

| | Oligonucleotide | Orien- | Product |
|---|---|---|---|
| Variant | sequence | tation | size |
| ENPP1 | GGATCATACTCAGGA | Forward | 321 |
| c.536A>G | AGACAGC | | |
| | TGGCCAATAGCCATG | Reverse | |
| | ACTCC | | |

OI, osteogenesis imperfecta; OPPG, osteoporosis-pseudoglioma syndrome

### Cloning of Complementary DNA

In case of patient 3, Total RNA from peripheral blood mononuclear cells was used for the synthesis of ENPP1 cDNA, using reverse transcription and the polymerase chain reaction (RT-PCR). ENPP1 cDNA and pT7blue T-vector (Merck, Darmstadt, Germany) were ligated before transformation into E. coli. Amplified vectors were purified with QIAprep Spin Miniprep kit (Qiagen, Redwood City, CA, USA) and subject to sequencing in both alleles.

Measurement of intact FGF23

Intact FGF23 was measured by Determinar CL FGF23 (CL), in accordance with the manufacturer's protocol (Minaris Medical, Tokyo, Japan). CL is a sandwich chemiluminescent enzyme immunoassay (CLEIA), using anti-human FGF23 mouse monoclonal antibodies. Using this method, the reference range of FGF23 is 16.1-49.3 pg/ml with cut-off values for FGF23-related hypophosphatemia of 30 μg/ml (Ito et al., 2021; Kato et al., 2021).

### Measurement of Plasma PPi

Plasma was collected from participants to measure the plasma pyrophosphate (PPi) concentrations. After plasma isolation, samples were filtered through a 30 kDa membrane (PALL, Port Washington, NY, USA) via centrifugation to remove platelets, and frozen at −80° C. within 1 hour of blood collection, for single use. Measurement of plasma [PPi] was performed using ATP sulfurylase as previously described with minor modifications (Jansen et al., 2013; Jansen et al., 2014). Luminescence signal was read by EnSpire Multimode Plate Reader (PerkinElmer, Waltham, MA, USA) at room temperature. Final plasma [PPi] were normalized by background subtracting [PPi] from appropriate controls. Reference ranges of [PPi] in healthy children and adolescents using the ATP sulfurylase method was recently reported to be 2,360 to 4,440 (Bernhard et al., 2022). These values are similar to prior standard ranges previously reported in healthy adults (O'Neill et al., 2010).

### Enzyme Kinetic Assay for ENPP1

Human mutations were engineered into the hENPP1-Fc construct using the Quikchange II-XL Site Directed Mutagenesis (Agilent Technologies, Santa Clara, CA, USA). After sequence verification, constructs were transfected into CHO-K1 cells using Lipofectamine 2000 (Thermo Fisher Scientific, Waltham, MA, USA). Forty-eight hours after transfection, 10 μL of supernatant was mixed with 90 μL of assay buffer containing 250 mM Tris pH 8.0, 500 mM NaCl, 0.05% Triton X-100, and 1 mM Thymidine 50-monophosphate p-nitrophenyl. The velocity of the p-nitrophenyl group liberated from the chromogenic substrate was reported as change at OD 405 nM/min, in replicates of at least 5 for each construct, and normalized to % WT.

### Statistical Analysis

PPi levels were presented as the mean±standard deviation (SD). Comparison of enzyme kinetic assay for ENPP1 between WT and mutations was analyzed by ANOVA. Significance was set at P<0.05. Data analysis was performed via GraphPad Prism version 6.05 for Windows (GraphPad Software, San Diego, CA, USA).

### Example 1: Identification of ENPP1 Haploinsufficiency in Patients

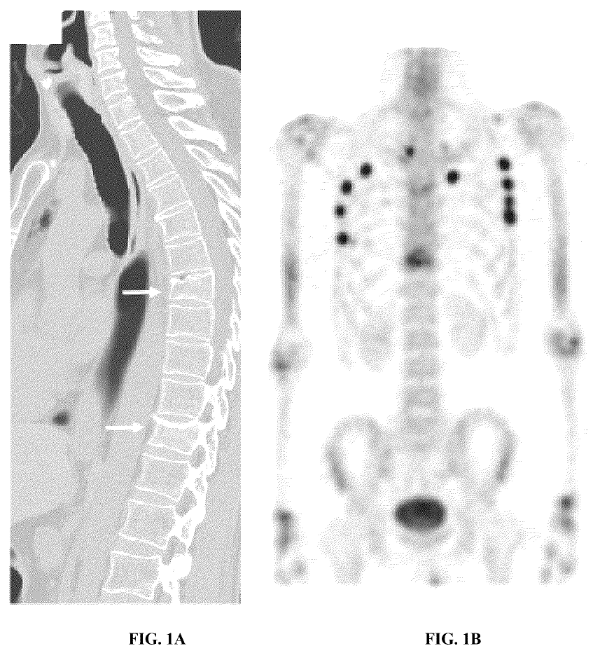

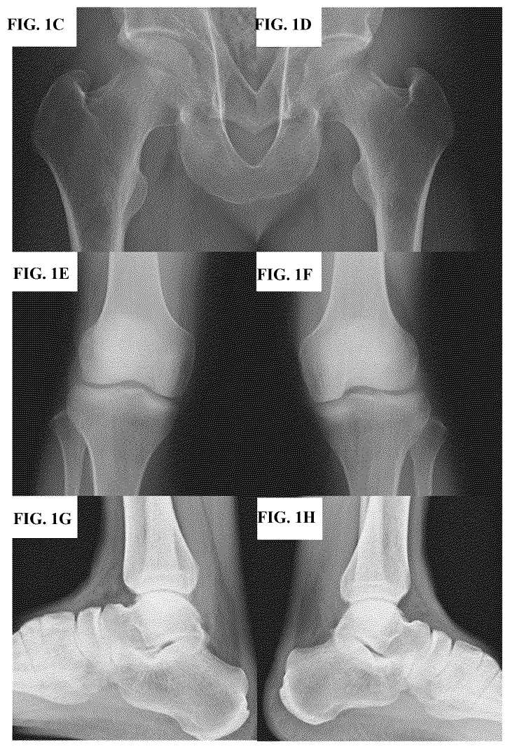

Patient 1 was a 47 year-old male with a history of fractures in the upper and lower extremities. He was a social drinker and had a 20 pack-year smoking history. He had no history of ureterolithiasis or malabsorption syndrome, no history of therapy associated with osteoporosis, and no familial history of osteoporosis. At the age of 46 he suffered back pain which was diagnosed as a spinal compression fracture, and also knee, wrist and ankle pain which began one month after the backache. He was referred to a local hospital, and dual-energy X-ray absorptiometry (DEXA) of the lumbar and proximal femurs showed a T-score of −3.8 and −2.6, respectively. CT scan of the spine demonstrated compression fracture at the 7th and 11th thoracic vertebrae (FIG. 1A).

Bone scintigraphy showed multiple uptake in the ribs, suggesting multiple fractures (FIG. 1B). No signs of ectopic ossifications were detected in the hip and knee joints, the Achilles tendons, or the paraspinal ligaments (FIGS. 1C-1H). The patient was diagnosed with early-onset osteoporosis, and laboratory examination to exclude a secondary osteoporosis and osteomalacia was performed. Although low-normal serum phosphorous along with slightly elevated serum alkaline phosphatase and bone alkaline phosphatase was detected at one local hospital, no abnormalities in serum calcium, phosphorous, or FGF23 were detected at another hospital (Supplementary Table 1).

Other endocrine function tests such as thyroid-stimulating hormone, free testosterone, and cortisol (measured in the morning) were within the normal limits (Supplementary Table 1). He was therefore referred to The University of Tokyo Hospitals for further evaluation, and blood chemistry was re-assessed (Table 3). Laboratory data showed normal levels of serum albumin, normal corrected calcium (8.8 mg/dL), low normal serum phosphorous (2.7 mg/dL, reference interval 2.7-4.6 mg/dL), normal FGF23 (28 pg/mL, reference interval 16.1-49.3 pg/mL), and low 25 hydroxy vitamin D (7.2 ng/mL).

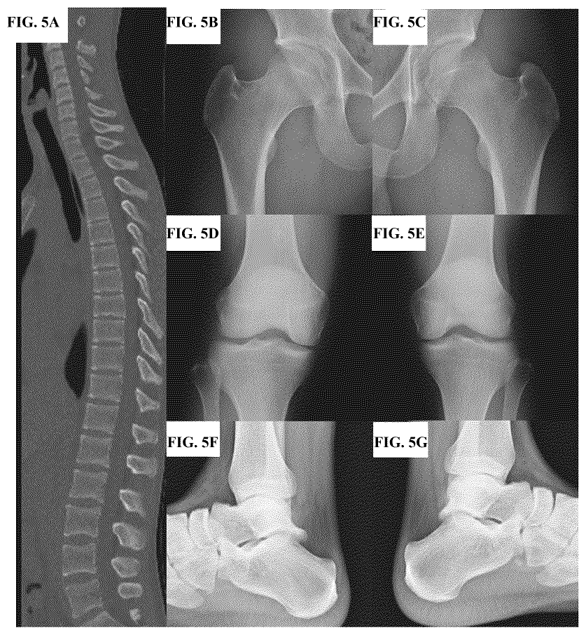

The diagnosis of a mild phenotype of OI or OPPG was considered, and mutational analysis in genes associated with OI or OPPG was performed (Table 2) without the identification of a pathogenic variant. Based on the serum analytes measured at the initial hospital (Supplementary Table 1) and the presence of low-normal serum phosphorous at our hospital, the diagnosis of a mild hypophosphatemic rickets was next considered, and mutational analysis of relevant genes performed, revealing a missense variant in ENPP1 (c.536A>G, p.Asn179Ser [N179S]) (Table 3). Direct sequencing of the detected variant in ENPP1 was next performed in the 18-year-old patient's son, revealing an identical heterozygous ENPP1 variant (FIG. 2A). Although the son exhibited low-normal BMD—low Z-scores in the lumbar (−1.6) and proximal femur (−0.8)—he had no history of spontaneous fractures or ectopic ossification in the spine, hip or knee joints, or in the Achilles tendons (FIGS. 5A-5G and Table 3).

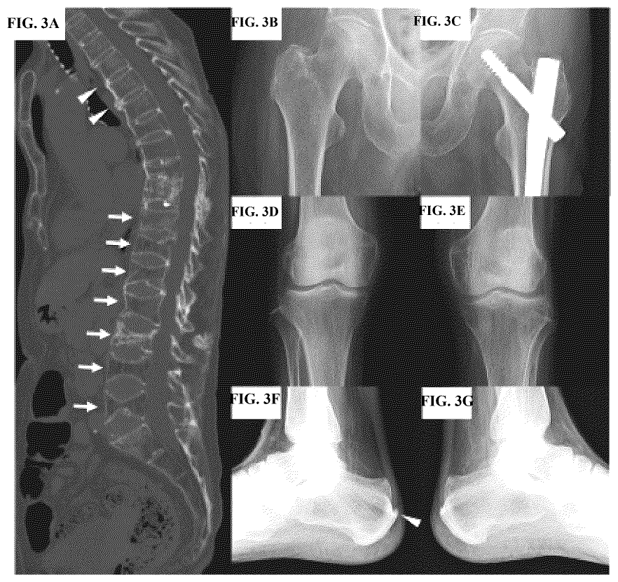

Patient 2 was a 77 year-old female was diagnosed with diffuse idiopathic skeletal hyperostosis (DISH) and referred to the University of Tokyo hospital when she suffered compression fractures in the spine. On evaluation, ossifications in paraspinal ligaments and multiple spinal compression fractures were observed by CT (FIG. 3A). In addition, slight ectopic ossifications were detected in the Achilles tendon (FIG. 3F), but the hip and knee joints were intact (FIGS. 3B, 3C, 3D, and 3E). The ossifications detected by spinal CT were not histologically evaluated. The patient's biochemical profile demonstrated low-normal serum phosphorous (3.1 mg/dL, reference interval 2.7-4.6 mg/dL) and high-normal FGF23 (43.3 pg/mL, reference interval 16.1-49.3 pg/mL). Due to the presence of ectopic ossifications in the paraspinal ligaments and the Achilles tendons, the diagnosis of a hereditary FGF23-related hypophosphatemia (specifically, ARHR or XLH) was considered. Testing of genes associated with hereditary hypophosphatemic rickets (Table 2) revealed the presence of a heterozygous missense variant in ENPP1 (c.1352A>G, p.Tyr451Cys [Y45IC]) (Table 3).

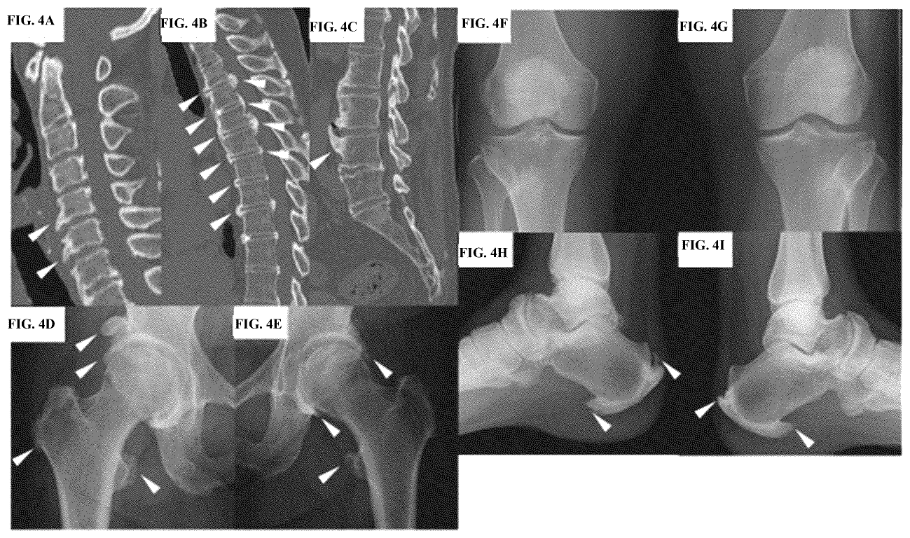

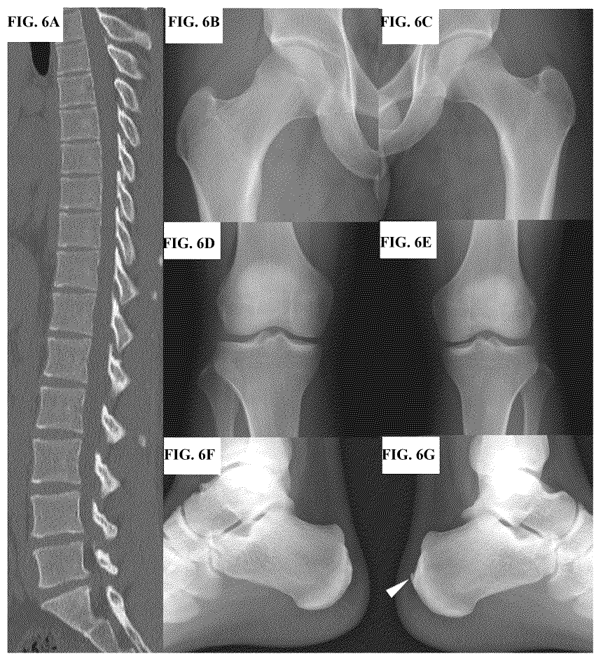

Patient 3 was a 54-year-old female visited a nearby hospital with complaint of pain in the hip, knee joints, and back. She subsequently exhibited ossifications of the anterior/posterior longitudinal ligament (OALL/OPLL), which were diagnosed as DISH, osteophytes around the bilateral hip joint, and enthesopathy in the bilateral Achilles tendons (FIGS. 4A-4I). Laboratory data showed low-normal serum phosphate (2.9 mg/dL) with high-normal serum FGF23 (38.4 pg/mL, reference range 16.1-49.3 pg/mL). The biochemical profile combined with the findings of the ectopic paraspinal ossifications lead to a conclusion of an FGF23-related hypophosphatemia, and genetic analyses (Table 2) revealed the same ENPP1 variants (c.536A>G and c.1352A>G) as those in patients 1 and 2 (Table 3). Sanger sequencing was next performed in her sons (aged 19 and 23 years), revealing heterozygous ENPP1 variants c.1352A>G in both (FIG. 2C). Significantly, both of these young adults exhibited calcific Achilles tendon enthesopathies without the presence of ectopic ossification in the spine or hip (FIGS. 6A-6G and 7A-7G).

SUPPLEMENTARY TABLE 1
Biochemical and hormonal data of case 1 before referral

| | RI, adults | Case 1 |
|---|---|---|
| Serum calcium (mg/dL) | 8.8-10.1 | 9.4 |
| Serum phosphate (mg/dL) | 2.7-4.6 | 2.8 |
| eGFR (mL/min/1.73 m²) | | |
| 25(OH)D (ng/mL) | | <4.0 |
| 1,25(OH)₂D (pg/mL) | 20-60 | 39.7 |
| ALP (U/L) | 38-113 | 152 |
| BAP (μg/L) | 3.7-20.9 | 25.6 |
| TRACP-5b (mU/dL) | 170-590 | 594 |
| iPTH (pg/mL) | 25.8-75.7 | 36 |
| FGF23 (pg/mL) | 16.1-49.3 | 28 |
| TSH (μIU/mL) | 0.38-4.31 | 2.46 |
| Free testosterone (pg/mL) | 4.7-21.6 | 5.6 |
| Cortisol (μg/dL) | 4.5-21.1 | 6.0 |

Abbreviations: RI, reference interval; eGFR, estimated glomerular filtration rate; 25(OH)D, 25-hydroxy vitamin D; 1,25(OH)₂D, 1,25-dihydroxy vitamin; ALP, alkaline phosphatase; BAP, bone alkaline phosphatase; TRACP-5b, Tartrate-resistant Acid Phosphatase 5b; iPTH, intact parathyroid hormone; FGF23, fibroblast growth factor 23; TSH, thyroid stimulating hormone.

TABLE 3
Demographical, clinical, and biochemical data in adults with monoallelic ENPP1
variant or compound heterozygous ENPP1 variant, c.536A > G or c.1352A > G.

| | | | Son of | | | Son of | Son of |
|---|---|---|---|---|---|---|---|
| | RI, adults | Case 1 | Case 1 | Case 2 | Case 3 | Case 3 (III-2) | Case 3 (III-3) |
| ENPP1 variant | | c.536A > G, | c.536A > G, | c.1352A > G | c.536 A > G/ | c.1352A > G | c.1352A > G |
| | | heterozygous | heterozygous | heterozygous | c.1352 A > G | heterozygous | heterozygous |
| Protein change | | p.Asn179Ser | p.Asa179Ser | p.Tyr451Cys | p.Asn179Ser/ | p.Tyr451Cys | p.Tyr431Cys |
| | | | | | p.Tyr451Cys | | |
| Demographical data | | | | | | | |
| Age (years)/Sex | | 48/M | 18/M | 78/F | 54/F | 23/M | 19/M |
| Height (cm/SD) | | 166/−0.9 | 177/1.2 | 146/−0.9 | 155/−0.4 | 167/−0.4 | 164/−2.0 |
| Clinical data | | | | | | | |
| Ectopic ossifications | | | | | | | |
| Paraspinal ligament | | No | No | Yes | Yes | No | No |
| Hip joints | | No | No | No | Yes | No | No |
| Knee joints | | No | No | No | No | No | No |
| Achilles tendon | | No | No | Yes | Yes | Yes | Yes |
| BMD (T-score/Z-score) | | | | | | | |
| Proximal Femur | | −2.6/−2.3 | NA/−0.8 | −3.5/−1.3 | 0.2/0.7 | NE | NE |
| Lumbar | | −3.8/−2.6 | NA/−1.6 | −3.3/−0.8 | 3.6/2.9 | NE | NE |
| Biochemical data | | | | | | | |
| Serum calcium (mg/L) | 8.8-10.1 | 8.8 | 9.9 | 9.6 | 9.3 | 9.6 | 10.0 |
| Serum phosphate (mg/L) | 2.7-4.6 | 2.7 | 3.7 | 3.1 | 2.9 | 3.4 | 3.2 |
| Plasma PPᵢ (nM) | 2360-4440 | 16463 ± 36 | NE | 1748 ± 45 | 1866 ± 82 | 1585 ± 56 | 1153 ± 31 |
| eGFR (mL/min/1.73 m²) | | 76.8 | 99 | 96.4 | 84.2 | 84.4 | 129.1 |
| 25(OH)D (ng/mL) | | 7.2 | 18.1 | 9.5 | 9.2 | 15.0 | 10.1 |
| 1.25(OH)₂D (pg/mL) | 20-60 | 40 | 31 | 26 | 48.1 | 68.3 | 65.9 |
| BAP (μg/L) | 3.7-20.9 | 13.5 | 13.9 | NE | 19.1 | 12.5 | 26.5 |
| TRACP-5b (mU/dL) | 170-590 (M) | 450 | 308 | 245 | 289 | 228 | 430 |
| | 120-420 (F) | | | | | | |
| iPTH (pg/mL) | 25.8-75.7 | 35 | 35 | 29 | 106 | 37.1 | 21.4 |
| FGF23 (pg/mL) | 16.1-49.3 | 28.0 | 34.0 | 43.3 | 38.4 | 39.0 | 43.2 |
| TPR (%) | >80 | 93 | 93 | NE | 96 | 97 | 97 |
| TmP/GFR (mg/dL) | 2.3-4.3 | 2.5 | 3.6 | NE | 2.8 | 3.3 | 3.1 |

Abbreviations: RI, reference interval; M, male; F, female; SD, standard deviation; BMD, bone mineral density; NA, not applicable; NE, not examined; PPᵢ, pyrophosphate; eGFR, estimated glomerular filtration rate; 25(OH)D, 25-hydroxy vitamin D; 1.25(OH)₂D, 1.25-dihydroxy vitamin; BAP, bone alkaline phosphatase; TRACP-5b, Tartrate-resistant Acid Phosphatase 5b; iPTH, intact parathyroid hormone; FGF23, fibroblast growth factor 23; TPR, tubular phosphate reabsorption.

### Example 2: Characterization of Effects of ENPP1 Haploinsufficiency in Patients

### Cloning of Complementary DNA for ENPP1 in Patient 3

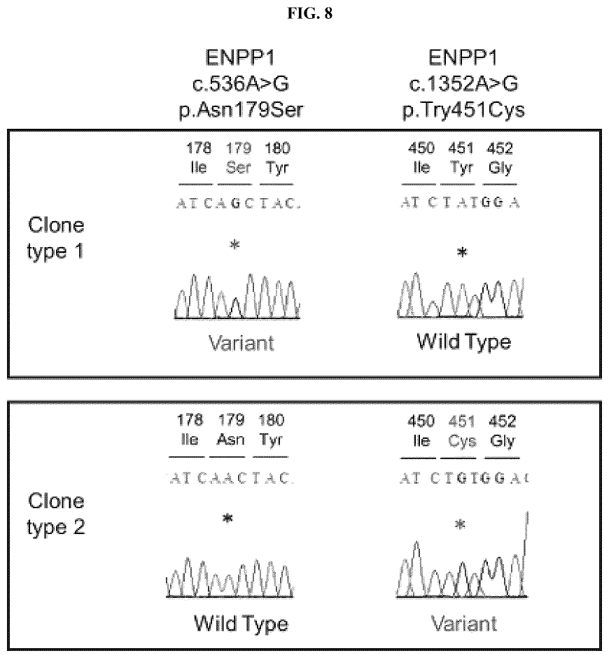

Complementary DNA for ENPP1 was cloned and sequenced to evaluate whether two ENPP1 variants were positioned in the same allele or in the opposite allele. Sequence data of each allele revealed that case 3 had compound heterozygosity for ENPP1 variants (FIG. 8).

### In Silico Prediction of Pathogenicity for the Detected ENPP1 Variants

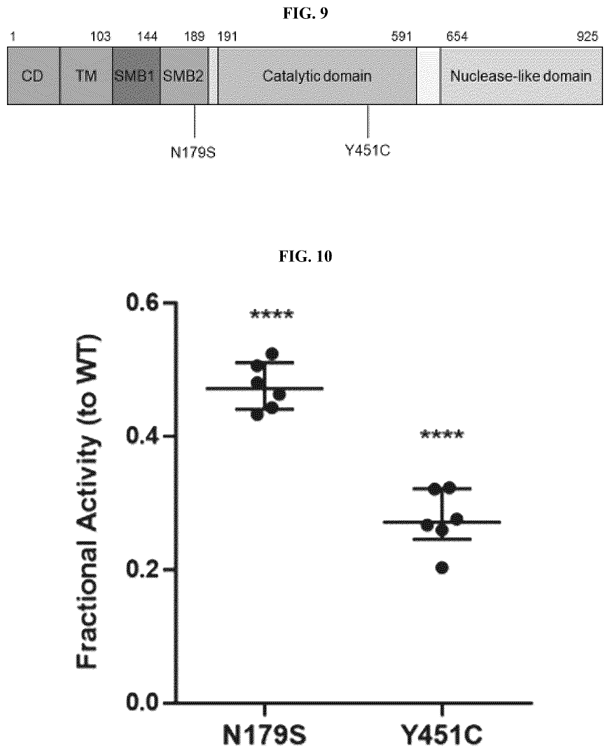

Allele frequencies of N179S and Y451C, which were detected in present cases, were reported to be 0.00010 and 0.00016 in the Genome Aggregation Database (GnomAD), respectively. However, in a whole-genome reference panel from 3552 general Japanese individuals constructed by the Tohoku Medical Megabank Organization (ToMMo), the allele frequencies of N179S and Y451C were reported to be 0.0071 and 0.0055, respectively (Table 4). Asn 179 is located in somatomedin B domain 2 (SMB2), which is crucial for protein dimerization and stability, while Tyr 451 is in the catalytic domain (FIG. 9), and both Asn 179 and Tyr 451 are highly conserved across all species. A combination of in silico tools described in the methods labelled the N179S and Y451C mutations as pathogenic (Table 4).

TABLE 4
Allele frequency and in silico predictions of ENPP1
variant, c.536A > G or c.1352A > G

| | | | In silico predictions | | |
|---|---|---|---|---|---|
| | Allele frequency | | | | Mutation |
| Variant | GnomAD | ToMMo | PolyPhen-2 | SIFT | Taster |
| c.536 | 0.00010 | 0.0071 | Probably | Damaging | Disease |
| A > G | | | damaging | | causing |
| c.1352 | 0.00016 | 0.0055 | Possibly | Damaging | Polymorphism |
| A > G | | | damaging | | |

### Measurement of Plasma PPi

Plasma PPi was measured in three probands (cases 1-3) and their family members. Patients with heterozygous ENPP1 variants exhibited plasma PPi levels between 1,000 to 2,000 nM (Table 3), and plasma PPi in the patient with biallelic ENPP1 variants exhibited similar concentrations of 1,866 nM.

### Enzyme Kinetic Assay for ENPP1

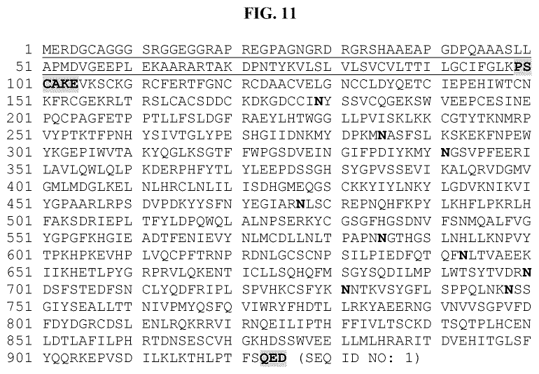

To further evaluate the effect of the mutations on ENPP1 catalytic activity, the enzymatic rate of all variants was evaluated in side by side in vitro assays compared to the WT ENPP1 isoform. The N179S and Y451C variants reduced the catalytic rate of ENPP1 55% and 70%, respectively, when compared to WT ENPP1 (FIG. 10). The N179S and Y451C variants were therefore classified as “likely pathogenic”, according to ACMG guideline (N179S: PM1+PP3+PS3, Y451C: PM1+PS3).

### Selected Results

The results indicate the presence of clinical manifestations of two cases of monoallelic ENPP1 deficiency and a case of compound biallelic heterozygous ENPP1 deficiency in primary subjects (patients 1-3), and an additional three cases of ENPP1 haploinsufficiency in the children of these subjects.

Patient 1 presented with early-onset osteoporosis, whereas patient 2 presented with hyperostosis of the spine and a presumptive diagnosis of DISH and was additionally found to be osteoporotic (as evidenced by compression fractures in spine, FIGS. 1A-1B and 3A-3G). Patient 3 possessed compound heterozygous ENPP1 mutations comprised of the individual mutations present in patients 1 and 2 such that the patient was ENPP1 homozygous deficient, and the patient exhibited prominent ossifications in the paraspinal ligaments as well as calcifications around the hip joints and bilateral calcific enthesopathies of the Achilles tendons (FIGS. 4A-4I).

Moreover, all of children who possessed monoallelic ENPP1 variants inherited from the primary patients exhibited musculoskeletal disease at a surprisingly young age, such as low bone mass in the 18-year-old son of patient 1 (Z score of −1.6 in the lumbar spine) and calcific enthesopathies in the Achilles tendons of the 19 and 23 year old children of patient 3. The segregation of genotype with phenotype further support the notion that ENPP1 deficiency plays a central role in the pathogenesis of the skeletal disorders described in the study.

Previously, evaluations of a family possessing homozygous ENPP1 mutations inducing GACI reported that the haploinsufficient ENPP1 carriers were asymptomatic, but possessed serum biochemistries of hypocalcemia and hypophosphatemia, suggesting a possible role for ENPP1 in calcium and phosphate homeostasis (Kotwal et al., 2020). In contrast, Oheim et al. reported ENPP1 haploinsufficiency in adult men with early-onset osteoporosis, a phenotype also present in an ENPP1 homozygous deficient mouse called ENPP1asj/asj (Oheim et al., 2020) supporting the notion that ENPP1 regulates mammalian bone mass. Indeed, the skeletal phenotype of patient 1 and her 18 year old son supports the association of ENPP1 haploinsufficiency with early onset osteoporosis.

The study extends the association of ENPP1 haploinsufficiency into patients with DISH. DISH is a systemic condition characterized by the ossification of ligaments and entheses, especially around the thoracic spine. Patients with DISH sometimes suffer pain and a reduced range of motion, and an increased risk of spinal fractures (Mader et al., 2013). Although the pathogenic mechanism is unknown, DISH is associated with older age (age over 50), male sex, obesity, hypertension, and diabetes mellitus (Kuperus et al., 2020)

In case of patient 2, she was aged over 50, but other risk factors for DISH were absent and instead the patient was found to be ENPP1 haploinsufficient. Given that clinical and preclinical studies in humans and mice have associated homozygous ENPP1 deficiency with spinal ligament ossification (Okawa et al., 1998; Nakamura et al., 1999; Saito et al., 2011; Hirao et al., 2016) the impaired ENPP1 activity leading to lowered plasma PPi is a risk factor for the progressive paraspinal ossifications and calcific enthesopathies present in this patient.

Both of the ENPP1 variants described (N179S and Y451C) are in highly conserved sequences located in regions of ENPP1 important for the dimerization and stability (Asn 179), and catalytic activity (Tyr 451). Furthermore, N179S and Y451C were found to be deleterious by multiple in silico tools (Table 4) and have not been reported as pathogenic to date.

In addition, N179S and Y451C reduced enzymatic activity by 55% and 70% compared to WT levels, respectively, which is similar to the residual enzymatic activity present in other pathogenic variants of ENPP1 (Kotwal et al., 2020; Oheim et al., 2020; Rutsch et al., 2003; Stella et al., 2016; Thumbigere-Math et al., 2018). Taken together, these findings support the finding that the ENPP1 variant(s) N179S and/or Y451C is/are responsible for the skeletal phenotypes and ectopic ossifications present in the three probands.

While the mechanism by which ENPP1 regulates musculoskeletal mineralization is yet to be fully understood, i.e., whether through catalytic effects or catalysis-independent protein signaling, the patients described in patients 1 and 2 both exhibited low plasma [PPi](1,646 nM and 1,748 nM, respectively, with reference range of 2,360-4,440 nM), consistent with a role for ENPP1 catalytic activity in the aberrant phenotypes observed. Both ENPP1 and ABCC6 deficiency leads to low plasma PPi levels (Lorenz-Depiereux et al., 2010; Levy-litan et al., 2010; Rutsch et al., 2001; Nitschke et al., 2012; Le Saux et al., 2000). Plasma PPi levels in ENPP1 haploinsufficient patients are intermediate between the PPi levels of those without ENPP1 deficiency and homozygous ENPP1 deficient patients (Kotwal et al., 2020; Oheim et al., 2020). PPi levels in our patients also fell within the 1,000 to 2,000 nM range, but unexpectedly the PPi concentration of patient 3 with compound heterozygous ENPP1 deficiency was similar to the haploinsufficient ENPP1 patients, suggesting the involvement of a compensatory mechanism (Kotwal et al., 2020).

Additionally, secondary hyperparathyroidism was observed in patient 3, a finding also observed in other patients with homozygous ENPP1 deficiency (Kotwal et al., 2020, and Capelli et al., 2015) and in murine models of ENPP1 deficiency. PTH is also elevated in other disorders induced by elevated FGF23, such as Hyp mice and humans with XLH and ARHR who develop secondary hyperparathyroidism due to impaired activation of vitamin D by the action of FGF23 (Carpenter JBMR, 2011). A similar mechanism may account for the secondary hyperparathyroidism in case 3, who was noted to be vitamin D deficient, as well as other cases of homozygous ENPP1 deficiency (Kotwal et al., 2020). As the patient described in patient 3 did not present with osteomalacia, the treatment with natural vitamin D instead of activated vitamin D would be, therefore, suitable to improve and prevent worsening of secondary hyperparathyroidism.

Although the patients in the study presented with normal intact FGF23, the levels were interpreted as elevated in the context of their low-normal serum phosphorous levels, raising possibility for a genetically induced phosphate wasting disorder. In this regard, the majority of patients described in the original reports of ARHR2 have high-normal or slightly elevated intact FGF23 in the context of low serum phosphorous. In Levy-Litan's description of 3 patients with ricket, 2 of the 3 had high-normal intact FGF23 (50 pg and 47 pg, with normal reference rage of 10-50 pg, (Levy-litan et al., 2010), and 5 of the 7 ARHR2 patients reported by Lorenz-Depiereux had intact FGF23 at either the upper range of normal or slightly elevated upon repeated measurement. These ranges were likewise interpreted as high in the context of the low phosphate values and the severe rickets phenotype exhibited by the patients.

Without wishing to be limited by any theory, the present studies indicate ENPP1 haploinsufficiency induces greater FGF23 elevations than homozygous ENPP1 deficiency, the normal and high-normal levels of intact FGF23 in the context of normal or low-normal serum phosphorous (2.7-3.1 mg/dL with a reference range of 2.7-4.6 mg/dL) raised the suspicion for a genetically induced hypophosphatemia, and genetic testing was initiated. The study indicates that increased circulating FGF23 and hypophosphatemia occurs in patients with calcifying enthesopathies in rare metabolic disorders such as ARHR, XLH, and hypophosphatemic tumoral calcinosis (Okawa et al., 1998; Nakamura et al., 1999; Saito et al., 2011; Hirao et al., 2016; Maulding et al., 2021; Rutsch et al., 2001; Cheng et al., 2005; Albright et al., 2015), and in the general medical population in patients with rapidly progressive forms of OPLL (Albright et al., 2015; Ferreira et al., 2021). In contrast, calcifying enthesopathies are not associated with FGF23-independent forms of hypophosphatemic rickets, such as SLC34A3 deficiency (Kotwal et al., 2020). FGF23 thus appears to be intimately related to the development of enthesopathy.

Prior transcriptome analysis performed in the whole bones of ENPP1 deficient mice reported that inactivating mutations of Enpp1 increased Fgf23 transcription and decreased Wnt10b and Wnt16 transcription (Maulding et al., 2021), suggesting that ENPP1 deficiency induces aberrant skeletal mineralization partly through impaired Wnt signaling. Patient 1 did not exhibit overt hypophosphatemic rickets with elevated FGF23 levels, and the bone resorption marker tartrate-resistant acid phosphatase 5b (TRACP-5b) was in the normal range suggesting the development of osteoporosis without high bone turnover—a finding consistent with an anabolic defect induced by defective Wnt signaling. The human observations are therefore consistent with the osteoporotic mechanisms suggested by transcriptome analysis in murine models of ENPP1 deficiency (Maulding et al., 2021)

Finally, it is important to note the differences in the allele frequency of the variants described in the study. The frequency of the allele detected in patient 2 (Y451C) is about 30 times higher in Japanese (ToMMo: 0.0055) than in all other races (GnomAD: 0.00016). Given the observation that monoallelic ENPP1 Y451C mutations are associated with OPLL, the differences in this variant frequency may explain the 10-40 times higher prevalence of OPLL in Japan (1.8-4.1%) than in the United States (0.12%) and Germany (0.1%) (Stapleton et al., 2011).

### Example 3: Generation of ENPP1 Fusion Proteins and Treatment of DISH Model Mice

### ENPP1 Generation

One example of an ENPP1 fusion protein is ENPP1-Fc. However, the exemplification of ENPP1-Fc can be applied to other ENNPP1 fusion proteins (such as ENPP1-Albumin) as set forth herein.

ENPP1-Fc is a recombinant fusion protein that contains the extracellular domains of human ENPP1 (soluble ENPP1) coupled with an Fc fragment of IgG1 (rhENPP1-Fc). The recombinant extracellular domains of ENPP1-Fc contain its catalytic activity and are identical to the native ENPP1 enzyme. ENPP1-Fc is a recombinant human protein produced in CHO cells via a fed batch cell culture process that is free of animal-derived components. T he molecular weight of the ENPP1-Fc dimer is approximately 290 kDa; ENPP1-Fc is highly glycosylated and has a pI of approximately 6.0. Like endogenous ENPP1, the primary substrate for ENPP1-Fc is ATP, which is cleaved to AMP and PPi.

In a specific embodiment, soluble ENPP1 protein was fused to a human Fc domain with a linker via a linker (comprising a leucine, isoleucine, and asparagine). Three ENPP1-Fc constructs are shown in Table 1 as SEQ ID NOs: 3, 4, and 5 as purified from CHO cells.

Purification of ENPP1-Fc could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. Following purification of the protein, the catalytic activity of the ENPP1-Fc protein could be evaluated using pNP-TMP as a chromogenic substrate.

Treatment of DISH Model Mice with the ENPP1 Agent

The most well-established murine model for spinal enthesopathy (DISH) is an Enpp1 deficient mouse referred to as ttw (‘tip toe walking mice’). ttw mice exhibit a severe myelopathy and extensive paraspinal ligament calcification and osteophyte formation. To evaluate the relationship between PPi the severity of enthesopathy, Achilles' tendons in 23-week-old Enpp1^{asj} mice, that were maintained on a regular chow diet, are examined.

The animals were dosed with either vehicle (PBS) or human ENPP1-Fc capable of normalizing plasma [PPi] for over a week after a subcutaneous sub-milligram dose. The protocols for administration of ENPP1-Fe, radiographic analysis and histological experiments are outlined in Ferreira et al., Musculoskeletal Comorbidities and Quality of Life in ENPP1-Deficient Adults and the Response of Enthesopathy to Enzyme Replacement Therapy in Murine Models. J Bone Miner Res, 2021). Optionally the experiments can be repeated using an ENPP1 agent (such as but not limited to ENPP1-Fc) that contains a bone targeting domain to better delivery and higher efficacy.

The ENPP1 agent (such as but not limited to ENPP1-Fc) is administered to Enpp1^{asj} mice at 0.3 mg/kg per week between weeks 2-23, and plasma PPi in dosed Enpp1^{asj} mice was measured. PPi is noted to be significantly increased, but not completely normalized, relative to WT pairs (1358 vs 2235 nM, respectively, Table 6).

TABLE 6
Plasma Analytes in 23-Week-Old Enpp1^{WT}
and Dosed and Undosed Enpp1^{asj/asj} Mice

| | | Enpp1^{wt} + | ENPP1^{asj} + | Enpp1^{asj} + |
|---|---|---|---|---|
| Parameter | Unit | PBS | PBS | BL-1118 |
| Calcium | mg/dL | 6.6 ± 0.5 | 7.0 ± 0.8 | 6.9 ± 0.4 |
| Phosphate | mg/dL | 6.4 ± 0.9 | 5.7 ± 0.5 | 5.5 ± 0.8 |
| PTH | pg/ml | 137 ± 80 | 229 ± 89 | 524 ± 248*(⬆) |
| FGF23 | pg/ml | 172 ± 28 | 439 ± 215*(⬆) | 614 + 167***(⬆) |
| PPi | nM | 2235 ± 621 | 51 ± 22**** (⬇) | 1358 ± 268**(⬇) |

The enthesopathy in the Achilles' tendons is analyzed both histologically and using custom MATLAB software to quantitate the red pixels in photomicrographs of alizarin-red stained sections. The Achilles tendons of vehicle treated Enpp1^{asj} mice is expected to reveal substantial calcifications throughout the length of the tendon, whereas tendon calcification is expected to be suppressed in Enpp1^{asj} mice treated with ENPP1-Fc.

Without being bound by theory, enthesopathy can be dependent on plasma PPi, and entheses can be prevented by elevating plasma PPi with an enzyme biologic such as an ENPP1 agent, such as ENPP1-Fc. The findings indicate that complete suppression of enthesopathy may be attainable upon dose escalation.

### Effect of PPi on DISH Model Mice

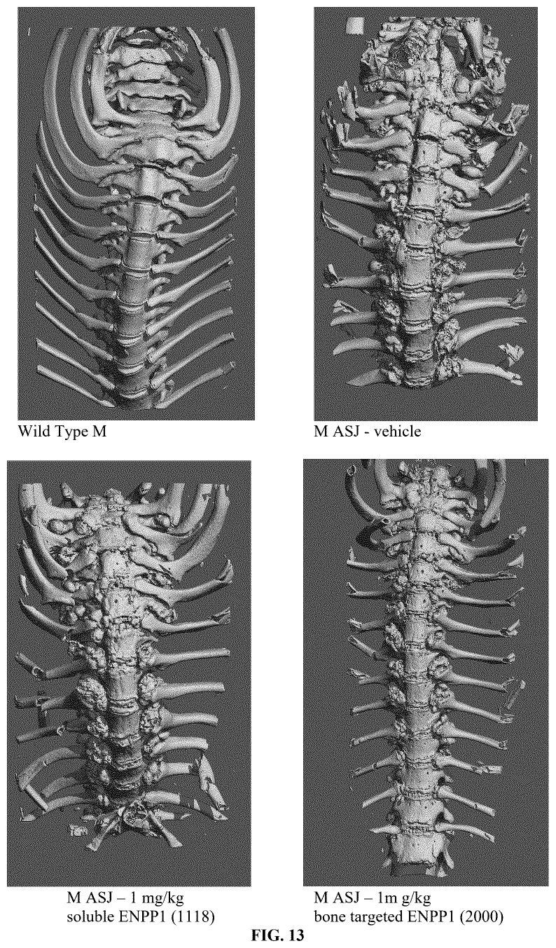

To evaluate the effect of plasma PPi on osteophytes formation, spinal fusion, and ossification in murine models of DISH, Enpp1^{asj} mice is dosed between weeks 3-17 with vehicle or 1 mg/kg ENPP1-Fc and their spines are analyzed by micro-CT. FIG. 12: The mice dosed with ENPP1-Fc exhibits higher plasma PPi levels (˜10 μm), several fold above WT levels.

The presence of elevated plasma PPi and reduction of paraspinal ossifications in the murine model of DISH treated with an ENPP1 agent such as ENPP1-Fc would indicate that such ENPP1 administration to human subjects with DISH would also attenuate paraspinal osteophytes, ankylosis, and spinal fusion.

### Example 4: Treatment Protocol

An ENPP1 agent (such as, but not limited to, ENPP1, ENPP1-X, ENPP1-Fc, and/or ENPP1-Fc-X, wherein X is a bone targeting tag as described elsewhere herein) is administered at one of the following selected doses: 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10.0 mg/kg, and/or fractions or multiples thereof. Administration is subcutaneous (SC) at least once or twice bimonthly, at least once or twice monthly, three times monthly, at least once or twice weekly.

The first dose of the ENPP1 agent may be administered on Day 1. On Days 8 and thereafter, the ENPP1 agent is administered to a subject at a selected dose of the ENPP1 agent mg/kg doses twice weekly. The dose may be administered at approximately the same time on each dosing day. The site of injection is alternated, with no site within 2 inches of any prior site of injection within the prior 2 weeks.

A selected dose of the ENPP1 agent is one of 0.2 mg/kg, 0.3 mg/kg, 0.4 mg/kg, 0.5 mg/kg, 0.6 mg/kg, or 1.8 mg/kg SC. Another selected dose of the ENPP1 agent by SC is one of 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10.0 mg/kg, and/or fractions or multiples thereof. The first dose of the ENPP1 agent may be administered on Day 1. After the first dose, a subject may be observed for 7 days to monitor safety and to collect PK samples. On Days 8 and thereafter, a subject receives a selected dose twice weekly. Administration of the ENPP1 agent at a selected dose is continued as considered appropriate by the medical professional.

A subject may receive 8 doses of the ENPP1 agent over the course of a 29 day period of time, for example, resulting in an exposure of 1.6 mg, 4.8 mg, and 14.4 mg per 29 days, respectively, for dose amounts of 0.2 mg/kg, 0.6 mg/kg, and 1.8 mg/kg. Or a subject may receive more or less than 8 doses, as considered appropriate by a medical profession.

Like the endogenous ENPP1 enzyme, the ENPP1 agent cleaves ATP to generate AMP and PPi, thereby increasing plasma PPi levels and into AMP which CD73 coverts rapidly to adenosine. Replacement of the endogenous human enzyme is intended to correct the inherent deficiency and allow for improved health and mitigation of clinical complications associated with ENPP1 Baseline patient, clinician, and caregiver outcomes.

### Example 5: Treatment of a Patient Having an ENPP1 Haploinsufficiency

The ENPP1 agent is administered to a patient identified as having an ENPP1 haploinsufficiency by subcutaneous injection on Day 1 and twice weekly starting on Day 8 using a select dose as follows.

TABLE 7

| | |
|---|---|
| 1 | 0.2 mg/kg |
| 2 | 0.6 mg/kg |
| 3 | 1.8 mg/kg |

The ENPP1 agent is administered at a selected dose of one of 0.2 mg/kg, 0.6 mg/kg, or 1.8 mg/kg SC at least twice weekly for a period of time determined by the medical professional. The patient's response to enzyme replacement is monitored as appropriate, as determined by the medical professional, e.g., by following a reduction in one or more symptoms of ENPP1 deficiency, and/or using guidance provided herein.

### Example 6: Treatment of Patient Diagnosed with DISH

DISH commonly involves the calcification of tendons and ligaments around the spine. Once the tendons and ligaments harden, parts of these tissues can turn into bone. This usually occurs where the tissue connects with the bone. As a result, bone spurs develop, which is an outgrowth of bone that develop along the edges of a bone. DISH commonly affects the upper part of the back and neck, known as the thoracic and cervical spine. However, DISH can also affect the shoulders, elbows, hands, knees, hips, heels, and/or ankles.

In certain embodiments, a subject who has been diagnosed with DISH is treated with the ENPP1 agent administered at a selected dose of one of 0.2 mg/kg, 0.6 mg/kg, or 1.8 mg/kg SC, IV, and/or IP at least twice weekly for a period of time determined by the medical professional.

In certain embodiments, a subject who has been diagnosed with DISH is treated with the ENPP1 agent administered at a selected dose of one of 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10.0 mg/kg, and/or fractions or multiples thereof, SC, IV, and/or IP at least twice weekly for a period of time determined by the medical professional.

DISH Patient response to enzyme replacement is monitored as appropriate, as determined by the medical professional, e.g., by following a reduction in one or more symptoms of DISH, using guidance provided herein.

Alternatively, a subject who is suspected of being at risk for DISH is treated with the ENPP1 agent by administration of a selected dose of the ENPP1 agent. In certain embodiments, common risk factors for developing DISH include but not limited to large waist circumference, BMI/Obesity, hyperinsulinemia, diabetes mellitus, hyperuricemia, dyslipidemia, hypertension, coronary artery disease, and/or gout. DISH can be asymptomatic and in those cases, diagnosis is usually made on the basis of the radiographic images. In certain embodiments, DISH is associated with older age (age over 50), male sex, obesity, hypertension, and/or diabetes mellitus.

Alternatively, subjects with radiographic evidence of DISH who lack clinical manifestations of the disease may be treated with the ENPP1 agent to prevent or minimize increased spinal fractures, reduced mobility, myelopathy, and/or pain, which are known phenotypes associated with DISH.

The following examples, in the context of the entire specification, provide guidance to determine treatment protocols and efficacy.

### Example 7: Biomarkers Associated with Bone Health

In addition to low plasma PPi, ENPP1 deficient patients are characterized biochemically by low serum phosphate, high urine phosphate, low renal TmP/GFR, normal calcium (Ca), low-normal urine Ca, normal 25-hydroxy Vitamin D (25 OH D), low-normal 1,25(OH)2D, high BAP, high intact FGF23, and normal and/or elevated PTH (IOF 2019).

Biomarkers that may be used in certain embodiments as additional determinants of bone health of a treated patient are set forth in Table 8.

TABLE 8
Clinical Intermediates and Biomarkers

| | Laboratory | Sample Type |
|---|---|---|
| Primary | Pyrophosphate (PPi) | Plasma or serum |
| Pharmacodynamic | Inorganic phosphate | Plasma or serum |
| Markers | FGF23 (intact) | Plasma or serum |
| | TmP/GFR | serum creatinine, serum |
| | | phosphate, urine |
| | | phosphate |
| | ALP, BALP, CTx, P1NP | serum, plasma, urine |

### Example 8: Efficacy of Treatment with the ENPP1 Agent

Treatment efficacy may be assessed by measuring plasma PPi as well as measuring other plasma analytes, such as FGF23, Pi, FGF23, Pi, TmP/GFR, serum alkaline phosphatase (ALP), bone-specific ALP (BALP), carboxy terminal cross-linked telopeptide of type I collagen (CTx), and/or procollagen type 1 N-terminal propeptide (P1NP). These analyte measurements may be used as a PD markers associated with ENPP1 Deficiency to determine the efficacy for the ENPP1 agent. Changes in these analytes may be described as changes from baseline and in a time-dependent manner over the course of treatment. Dose linearity of PK and PD parameters also may be assessed.

Changes from baseline in plasma PPi levels, FGF23 levels and Urinary phosphorus excretion per creatinine clearance may be analyzed using a t test of paired differences.

### Example 9: Drug Concentration Measurements

In addition, blood samples may be obtained from a patient for measurement of the ENPP1 agent concentration in plasma and subsequent determination of PK parameters following the first dose (i.e. single dose) and at/after multiple doses (i.e. steady-state).

### Example 10: Immunogenicity (Anti-Drug Antibodies)

If desired, immunogenicity to the ENPP1 agent may be measured using anti-drug antibodies (ADA). Immunogenicity testing can utilize a multi-tiered approach; if ADA are detected in the initial screen, a confirmatory test may be run to determine specificity. Samples may also be used to assess and further establish assays for specificity confirmation (i.e. titer) and neutralizing antibodies.

### Example 11: Pharmacokinetic, Pharmacodynamic, and Exploratory Biomarker Analyses

Pharmacokinetic analysis may be performed on the PK population, and PK parameters of the ENPP1 agent may be summarized by treatment with descriptive statistics. Dose linearity of PK and PD parameters may also be assessed. PK/PD analyses, immunogenicity analyses; and exploratory biomarker analyses may be determined.

### Example 12: Additional Determinators of Efficacy

Although restoring a normal level of PPi is the primary indicator of efficacy of treatment using the ENPP1 agent, other physical measurements also may be used, if desired to assist in determining treatment efficacy. These include one or more of the following.

### 1. Radiography and Imaging

a. X-Rays for Skeletal Severity. Standard X-rays may be obtained to detect rachitic skeletal deformities. Obtain X-rays may be obtained, for example, on the wrists and knees.
b. DEXA Scan. DEXA scans may be used to evaluate changes in bone density.
c. Positron Emission Tomography. Computed Tomography. Baseline Na18F-PET/HRpQCT (or HR-CT) may be a full body scan done within 1 month of first dose of the ENPP1 agent to measure calcification of arteries and organs and skeletal abnormalities at baseline and for future interventional assessments. The Na¹⁸F-PET measures bone turnover as well as microcalcification of the arteries. High-resolution quantitative computed tomography (HRQCT) or HR-CT can determine bone microstructure at the non-dominant distal radius and tibia. Standard bone geometric parameters are calculated.
d. Doppler Echocardiogram. A baseline echocardiogram may be obtained within 3 days prior to a first dose of the ENPP1 agent. Doppler echo may be used to measure heart function [LVEF, blood flow] calcification of heart and valves, and arterial stiffness.
e. Optical Coherence Tomography. Optical coherence tomography may be used to visualize neointimal proliferation.
f. Peripheral Arterial Tonometry. Peripheral arterial tonometry (PAT) may be used to assess digital pulse wave amplitude (PWA), which corresponds to digital volume variation.
g. Renal Ultrasound. Renal ultrasound may be used, for example, within 1 week of starting ENPP1 agent, to measure renal calcification.
h. Bone Histomorphology and Bone Biopsy. Bone biopsy may be performed as a baseline measurement. Tetracycline loading for 10 days prior to bone biopsy is preferred.

### 2. Walking Ability

Walk tests may be used as a submaximal exercise measurement to measure functional capacity in ambulatory patients combining cardiopulmonary, neuromuscular, and musculoskeletal functions. The 6 Minute Walk Test (6MWT) was originally developed by the American Thoracic Society (ATS 2002) for use with adults, and is now commonly used in both adult and pediatric populations (Mylius et al., 2016), and with children with neuromuscular diseases such as spinal muscular atrophy (Montes et al., 2018), Duchenne muscular dystrophy (McDonald et al., 2013), and infantile-onset Pompe disease (van der Meijden et al., 2018). The 2 Minute Walk Test (2MWT) is included in the NIH Toolbox and is increasingly being used to measure the same properties.

The 6MWT and the 2MWT may be administered to the patient before and during treatment at the discretion of the healthcare provider. If a subject is unable to complete at least the 2MWT at baseline, additional assessments during treatment may be left to a healthcare provider's discretion. Resting heart rate is obtained prior to the test and post-test. Distance walked in the first 2 minutes of the 6MWT and the full 6 minutes may be recorded. The distances walked in 2 minutes and 6 minutes may be compared to age- and sex-matched normative data (percent predicted values).

### 3. Dynamometry

Strength may be assessed using dynamometry before and/or during treatment at the discretion of the healthcare provider. Hand-held dynamometry is a direct measurement of strength commonly used in both children and adults. Muscle groups that may be assessed include: shoulder abduction, shoulder flexion, elbow flexion, elbow extension, hip abduction, hip flexion, hip extension, and knee extension. Each muscle group may be measured 2 times bilaterally.
a. Grip Strength. Grip strength may be measured using a grip strength dynamometer before and/or during treatment at the discretion of the healthcare provider. Equipment and assessor instructions may be standardized across sites. Grip may be assessed bilaterally with 1 practice and 1 maximal force measures taken for each hand and results may be compared to age and gender matched normative data (when available).
b. Range of Motion. Range of Motion may be assessed using a goniometer, an instrument that tests the angle of joints and measures the degree of movement at a joint. The stationary arm of the goniometer is aligned with the specified bony landmark on the stationary body segment, and the moving arm of the goniometer is aligned with the specified bony landmark of the limb that is moving. The fulcrum of the goniometer is specified for each motion measured using axis of motion and bony landmarks. Range of motion may be assessed for one or more of the following: shoulder abduction, shoulder flexion, elbow flexion, elbow extension, hip abduction, hip flexion, hip extension, and knee extension.

### 4. Hearing Testing

Moderate hearing loss has been associated with ARHR2 (Brachet et al 2014, Steichen-Gersdorf et al 2015). Baseline hearing may be determined by one or more of: Physical exam and otoscopy, Immittance audiometry (commonly called tympanometry), Pure Tone Audiometry (PTA) with frequencies up to 8 kHz if possible. (If there is a PTA threshold of >15 dB, the subject should also undergo bone conduction testing.), High Frequency Audiometry (HFA), with frequencies up to 16 kHz.

### 5. Clinician Global Impression Scales

The Clinical Global Impression (CGI-S) scales were developed for use in National Institute of Mental Health-sponsored clinical studies to provide a brief, stand-alone assessment of the clinician's view of the patient's global functioning prior to and after initiating a study medication (Guy 1976). The CGI provides an overall clinician-determined summary measure that considers all available information, including knowledge of the patient's history, psychosocial circumstances, symptoms, behavior, and the impact of the symptoms on the patient's ability to function. The CGI-S may be administered before and/or during treatment at the discretion of the healthcare provider and provides a global assessment of change using a seven-point scale ranging from −3 (severe worsening) to +3 (significant improvement).

### 6. Gross Motor Function Classification System—Expanded and Revised

The Gross Motor Classification System—Expanded and Revised (GMFCS—E and R) may be administered before and/or during treatment at the discretion of the healthcare provider. The GMFCS—E and R classifies patient-initiated movement with an emphasis on mobility on a scale from 1 to 5.

### 7. Patient Reported Outcomes Measurement Information Systems

The Patient Reported Outcomes Measurement Information Systems (PROMIS) consists of a variety of questionnaires developed by the National Institutes of Health (NIH) to evaluate physical, mental, and social well-being from the patient perspective (www dot healthmeasures dot net). These questionnaires have been used in clinical studies in people with chronic health conditions such as X-linked hypophosphatemia, arthritis, multiple sclerosis, and neurofibromatosis. Each questionnaire contains 8 to 10 items which are rated by the participant on a 5-point Likert scale ranging from 1 (never) to 5 (always). Scores are summed for each questionnaire, with high scores indicating more of the domain being measured (e.g. more fatigue, more physical function). Raw scores are converted to T-Scores based on a mean of 50 and a standard deviation of 10, allowing comparison of the study sample to the general population. PROMIS Scales may include the Pain Interference (short form 8a), Pain Intensity (version 3a), Physical Function—Upper Extremity (custom short form), Physical Function—Mobility (short form 13a FACIT Fatigue), Fatigue (short form), and Cognitive Impact (short form 8a) and may be administered before and/or during treatment at the discretion of the healthcare provider. These assessments may be completed by the subject without assistance.

### 8. Caregiver Global Impression Scales

The Caregiver Global Impression of Status may be administered to the patient's caregiver before and/or during treatment at the discretion of the healthcare provider. The Caregiver Global Impression of Change provides a global assessment of change using a seven-point scale ranging from −3 (severe worsening) to +3 (significant improvement).

### 9. Western Ontario and McMaster University Osteoarthritis Index

The WOMAC is a patient-reported outcome used to assess activities of daily living, functional mobility, gait, general health, pain, and quality of life in patients with hip or knee pain (www dot sralab dot org). The assessment consists of 24-items and takes approximately 12 minutes to administer. The WOMAC may be administered before and/or during treatment at the discretion of the healthcare provider. The assessment may be completed by the subject without assistance.

### Example 13: Comparison of Soluble (Construct 1118, SEQ ID NO: 118) and Bone Targeted (Construct 2000, SEQ ID NO: 119) ENPP1-Fc on Cervical Spinal and Hearing Phenotype

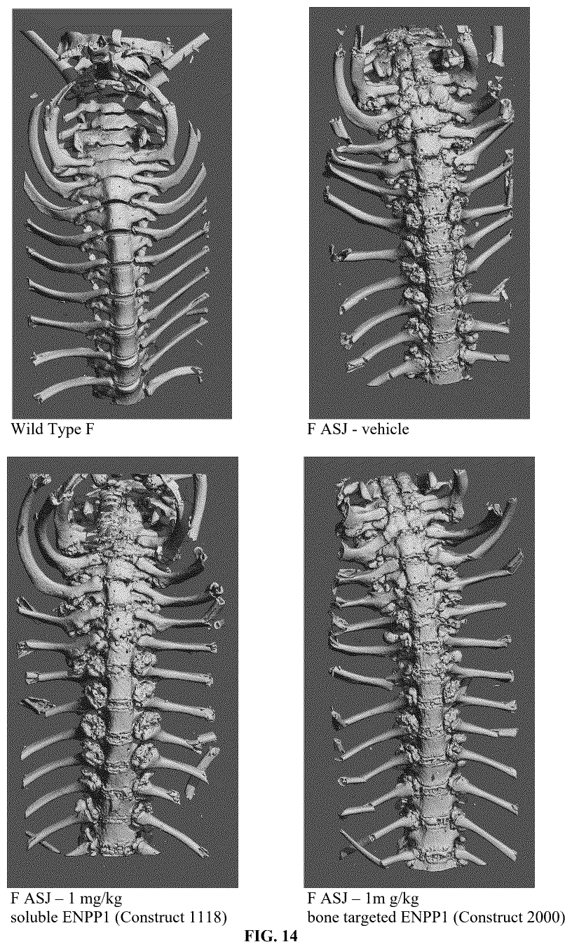

FIG. 13 shows response of paraspinal osteophytes and ankylosis in 17-week old WT and 17-week old Enpp1^{asj} male mice dosed with weekly indicated subcutaneous doses of vehicle or ENPP1 Constructs #1118 and #2000. Construct #1118 was dosed once a week beginning week 3, while Construct #2000 was dosed once a week beginning week 5. Micro-CT images demonstrate attenuation of paraspinal osteophytes and ankylosis preferentially in Construct #2000 dosed Enpp1^{asj} male mice.

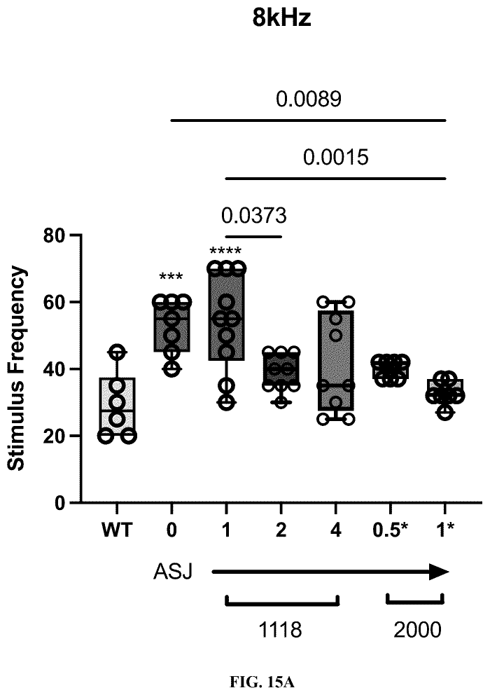

FIG. 14 shows response of paraspinal osteophytes and ankylosis in 17-week old WT and 17-week old Enpp1^{asj} female mice dosed with weekly indicated subcutaneous doses of vehicle or ENPP1 constructs #1118 and #2000. Construct #1118 was dosed once a week beginning week 3, while Construct #2000 was dosed once a week beginning week 5. Micro-CT images demonstrate attenuation of paraspinal osteophytes and ankylosis preferentially in Construct #2000 dosed Enpp1^{asj} female mice.

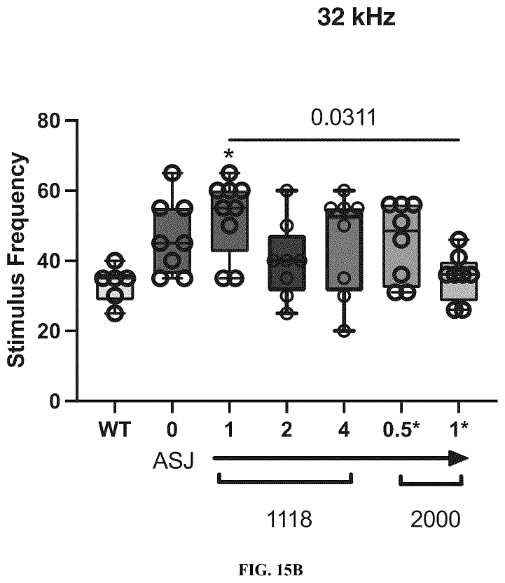

FIGS. 15A-15B shows auditory brain stem response in 17-week old WT and 17-week old Enpp1^{asj} mice dosed with weekly indicated subcutaneous doses of vehicle or ENPP1 constructs #1118 and #2000. Construct #1118 was dosed once a week beginning week 3, while Construct #2000 was dosed once a week beginning week 5. Stimulus frequency measurements demonstrate the prevention of hearing loss in the low frequency (8 kHz) range by weekly doses of 2 mg/Kg ENPP1 construct #1118, and at weekly doses of 0.5 and 1 mg/Kg of ENPP1 Construct #2000. Improvement in hearing in ENPP1 deficient animals is also noted in high frequency range (32 kHz) preferentially in ENPP1 Construct #2000 dosed at weekly doses of 1 mg/Kg.

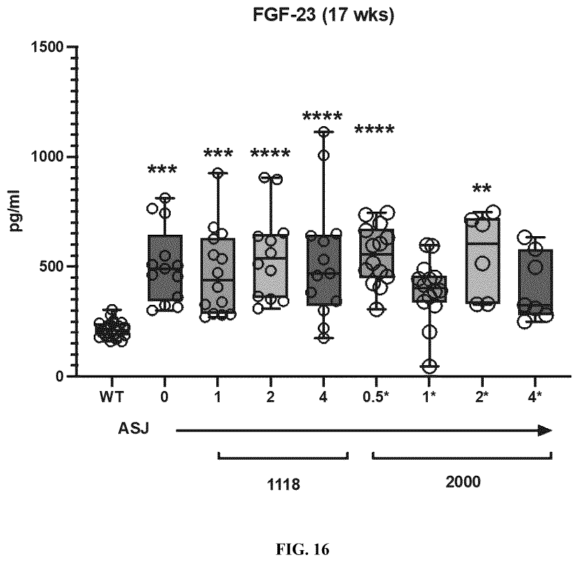

FIG. 16 shows the intact FGF23 levels of WT and 17-week old ENPP1^{asj} mice with the weekly indicated subcutaneous doses of vehicle or ENPP1 Constructs #1118 and #2000. Construct #1118 was dosed once a week beginning week 3, while Construct #2000 was dosed once a week beginning week 5. The data demonstrates suppression of intact FGF23 preferentially in ENPP1 Construct #2000 when dosed at 1 mg/Kg and 4 mg/Kg per week. Statistical significance was assessed by an ANOVA Kruskal-Wallis test followed by Dunn's post hoc analysis to evaluate differences with WT (one-way ANOVA). Statistical significance is denoted by p values with the notation: *p<0.05, **p<0.01, ***p<0.001, ****p<0.0001.

### REFERENCES

1. Lorenz-Depiereux B, Schnabel D, Tiosano D, et al. Loss-of-Function ENPP1 Mutations Cause Both Generalized Arterial Calcification of Infancy and Autosomal-Recessive Hypophosphatemic Rickets. Am J Hum Genet. 2010; 86:267-272.
2. Levy-litan V, Hershkovitz E, Avizov L, et al. Autosomal-Recessive Hypophosphatemic Rickets Is Associated with an Inactivation Mutation in the ENPP1 Gene. Am J Hum Genet. 2010; 86:273-278.
3. Goding J W, Grobben B, Slegers H. Physiological and pathophysiological functions of the ecto-nucleotide pyrophosphatase/phosphodiesterase family. Biochim Biophys Acta—Mol Basis Dis. 2003; 1638:1-19.
4. Okawa A, Nakamura I, Goto S, et al. Mutation in Npps in a mouse model of ossification of the posterior longitudinal ligament of the spine. Nat Genet. 1998; 19:271-273.
5. Nakamura I, Ikegawa S, Okawa A, et al. Association of the human NPPS gene with ossification of the posterior longitudinal ligament of the spine (OPLL). Hum Genet. 1999; 104:492-497.
6. Saito T, Shimizu Y, Hori M, et al. Case Report A patient with hypophosphatemic rickets and ossification of posterior longitudinal ligament caused by a novel homozygous mutation in ENPP1 gene. Bone. 2011; 49:913-916.
7. Hirao Y, Chikuda H, Oshima Y, et al. Extensive ossification of the paraspinal ligaments in a patient with vitamin D-resistant rickets: Case report with literature review. Int J Surg Case Rep. 2016; 27:125-128.
8. Maulding N D, Kavanagh D, Zimmerman K, et al. Genetic pathways disrupted by ENPP1 deficiency provide insight into mechanisms of osteoporosis, osteomalacia, and paradoxical mineralization. Bone. 2021; 142:115656.
9. Rutsch F, Vaingankar S, Johnson K, et al. PC-1 nucleoside triphosphate pyrophosphohydrolase deficiency in idiopathic infantile arterial calcification. Am J Pathol. 2001; 158:543-554.
10. Cheng K S, Chen M R, Ruf N, et al. Generalized arterial calcification of infancy: Different clinical courses in two affected siblings. Am J Med Genet. 2005; 136 A:210-213.
11. Albright R A, Stabach P, Cao W, et al. ENPP1-Fc prevents mortality and vascular calcifications in rodent model of generalized arterial calcification of infancy. Nat Commun. 2015; 6:1-11.
12. Ferreira C R, Kavanagh D, Oheim R, et al. Response of the ENPP1-Deficient Skeletal Phenotype to Oral Phosphate Supplementation and/or Enzyme Replacement Therapy: Comparative Studies in Humans and Mice. J Bone Miner Res. 2021; 36:942-955.
13. Ferreira C R, Ansh J, Nester C, et al. Musculoskeletal comorbidities and quality of life in ENPP1-deficient adults and the response of enthesopathy to enzyme replacement therapy in murine models. J Bone Miner Res. 2021. doi: 10.1002/jbmr.4487.
14. Kotwal A, Ferrer A, Kumar R, et al. Clinical and Biochemical Phenotypes in a Family With ENPP1 Mutations. J Bone Miner Res. 2020; 35:662-670.
15. Oheim R, Zimmerman K, Maulding N D, et al. Human Heterozygous ENPP1 Deficiency Is Associated With Early Onset Osteoporosis, a Phenotype Recapitulated in a Mouse Model of Enpp1 Deficiency. J Bone Miner Res. 2020; 35:528-539.
16. Fujiki R, Ikeda M, Yoshida A, et al. Assessing the Accuracy of Variant Detection in Cost-Effective Gene Panel Testing by Next-Generation Sequencing. J Mol Diagnostics. 2018; 20:572-582.
17. Adzhubei I A, Schmidt S, Peshkin L, et al. A method and server for predicting damaging missense mutations. Nat Methods. 2010; 7:248-249.
18. Kumar P, Henikoff S, Ng P C. Predicting the effects of coding non-synonymous variants on protein function using the SIFT algorithm. Nat Protoc. 2009; 4:1073-1082.
19. Schwarz J M, Rödelsperger C, Schuelke M, et al. MutationTaster evaluates disease-causing potential of sequence alterations. Nat Methods. 2010; 7:575-576.
20. Ito N, Kubota T, Kitanaka S, et al. Clinical performance of a novel chemiluminescent enzyme immunoassay for FGF23. J Bone Miner Metab. 2021. doi:10.1007/s00774-021-01250-1
21. Kato H, Hidaka N, Koga M, et al. Performance evaluation of the new chemiluminescent intact FGF23 assay relative to the existing assay system. J Bone Miner Metab. 2021. doi:10.1007/s00774-021-01258-7
22. Jansen R S, Küçükosmanoǧlu A, De Haas M, et al. ABCC6 prevents ectopic mineralization seen in pseudoxanthoma elasticum by inducing cellular nucleotide release.

### Proc Natl Acad Sci USA. 2013; 110:20206-20211.

23. Jansen R S, Duijst S, Mahakena S, et al. ABCC6-mediated ATP secretion by the liver is the main source of the mineralization inhibitor inorganic pyrophosphate in the systemic circulation—Brief report. Arterioscler Thromb Vasc Biol. 2014; 34:1985-1989.
24. Lek M, Karczewski K J, Minikel E V., et al. Analysis of protein-coding genetic variation in 60,706 humans. Nature. 2016; 536:285-291.
25. Kuriyama S, Yaegashi N, Nagami F, et al. The Tohoku Medical Megabank Project: Design and mission. J Epidemiol. 2016; 26:493-511.
26. Richards S, Aziz N, Bale S, et al. Standards and guidelines for the interpretation of sequence variants: a joint consensus recommendation of the American College of Medical Genetics and Genomics and the Association for Molecular Pathology. Genet Med. 2015; 17:405-424.
27. Rutsch F, Ruf N, Vaingankar S, et al. Mutations in ENPP1 are associated with “idiopathic” infantile arterial calcification. Nat Genet. 2003; 34:379-381.
28. Stella J, Buers I, van de Wetering K, et al. Effects of Different Variants in the ENPP1 Gene on the Functional Properties of Ectonucleotide Pyrophosphatase/Phosphodiesterase Family Member 1. Hum Mutat. 2016; 37:1190-1201.
29. Thumbigere-Math V, Algadi A, Chalmers N I, et al. Hypercementosis Associated with ENPP1 Mutations and GACI. J Dent Res. 2018; 97:432-441.
30. Stapleton C J, Pham M H, Attenello F J, et al. Ossification of the posterior longitudinal ligament: Genetics and pathophysiology. Neurosurg Focus. 2011; 30:9-12.
31. Resnick D, Shaul S R, Robins J M. Diffuse idiopathic skeletal hyperostosis (DISH): Forestier's disease with extraspinal manifestations. Radiology. 1975; 115:513-524.
32. Mader R, Verlaan J J, Buskila D. Diffuse idiopathic skeletal hyperostosis: Clinical features and pathogenic mechanisms. Nat Rev Rheumatol. 2013; 9:741-750.
33. Kuperus J S, Mohamed Hoesein F A A, de Jong P A, et al. Diffuse idiopathic skeletal hyperostosis: Etiology and clinical relevance. Best Pract Res Clin Rheumatol. 2020; 34:101527.
34. Rashdan N A, Rutsch F, Kempf H, et al. New perspectives on rare connective tissue calcifying diseases. Curr Opin Pharmacol. 2016; 28:14-23.
35. Whyte M P. Hypophosphatasia: Enzyme Replacement Therapy Brings New Opportunities and New Challenges. J Bone Miner Res. 2017; 32:667-675.
36. Nitschke Y, Baujat G, Botschen U, et al. Generalized arterial calcification of infancy and pseudoxanthoma elasticum can be caused by mutations in either ENPP1 or ABCC6. Am J Hum Genet. 2012; 90:25-39.
37. Le Saux O, Urban Z, Tschuch C, et al. Mutations in a gene encoding an ABC transporter cause pseudoxanthoma elasticum. Nat Genet. 2000; 25:223-227.
38. Kawaguchi Y, Kitajima I, Nakano M, et al. Increase of the Serum FGF-23 in Ossification of the Posterior Longitudinal Ligament. Glob Spine J. 2019; 9:492-498.
39. Kato H, Koga M, Kinoshita Y, et al. Incidence of Complications in 25 Adult Patients with X-linked Hypophosphatemia. J Clin Endocrinol Metab. 2021; 106:E3682-E3692.
40. Bernhard, E., et al., A Reference Range for Plasma Levels of Inorganic Pyrophosphate in Children Using the ATP Sulfurylase Method. J Clin Endocrinol Metab, 2022. 107(1): p. 109-118.
41. O'Neill, W. C., M. K. Sigrist, and C. W. McIntyre, Plasma pyrophosphate and vascular calcification in chronic kidney disease. Nephrol Dial Transplant, 2010. 25(1): p. 187-91.
42. Capelli, S., et al., Clinical and molecular heterogeneity in a large series of patients with hypophosphatemic rickets. Bone, 2015. 79: p. 143-9.
43. Chalmers, J., Enthesopathy as the presenting feature of X-linked hypophosphatemia.

A case report. Acta Orthop Scand, 1993. 64(2): p. 221-3.
44. Hardy, D. C., et al., X-linked hypophosphatemia in adults: prevalence of skeletal radiographic and scintigraphic features. Radiology, 1989. 171(2): p. 403-14.
45. Liang, G., et al., Survey of the enthesopathy of X-linked hypophosphatemia and its characterization in Hyp mice. Calcif Tissue Int, 2009. 85(3): p. 235-46.
46. Ramonda, R., et al., [The enthesopathy of vitamin D-resistant osteomalacia in adults]. Reumatismo, 2005. 57(1): p. 52-6.
47. Reid, I. R., et al., X-linked hypophosphatemia: a clinical, biochemical, and histopathologic assessment of morbidity in adults. Medicine (Baltimore), 1989. 68(6): p. 336-52.
48. Ferreira, C. R., et al., Musculoskeletal Comorbidities and Quality of Life in ENPP1-Deficient Adults and the Response of Enthesopathy to Enzyme Replacement Therapy in Murine Models. J Bone Miner Res, 2021.
49. Karaplis, A. C., et al., Mineralizing enthesopathy is a common feature of renal phosphate-wasting disorders attributed to FGF23 and is exacerbated by standard therapy in hyp mice. Endocrinology, 2012. 153(12): p. 5906-17.
50. Gafni, R., et al., SUN-364: Enthesophytes are a common feature of FGF23-mediated hypophosphatemia due to tumor-induced osteomalacia. J Endocrine Soc, 2020. 4-(Supplement 1).
51. Kawaguchi, Y., et al., Serum biomarkers in patients with ossification of the posterior longitudinal ligament (OPLL): Inflammation in OPLL. PLoS One, 2017. 12(5): p. e0174881.
52. Kawaguchi, Y., et al., Increase of the Serum FGF-23 in Ossification of the Posterior Longitudinal Ligament. Global Spine J, 2019. 9(5): p. 492-498.
53. Chen, A., et al., Description of 5 Novel SLC34A3/NPT2c Mutations Causing Hereditary Hypophosphatemic Rickets With Hypercalciuria. Kidney Int Rep, 2019. 4(8): p. 1179-1186.

### Additional References for Examples 3-12

ATS. American Thoracic Society Statement: Guidelines for the six-minute walk test. AmJRespir, CritCare Med. 2002 2002; 166(1):111-7.
Brachet C, Mansbach A L, Clerckx A, Deltenre P, Heinrichs C. Hearing loss is part of the clinical picture of ENPP1 loss of function mutation. Horm Res Paediatr. 2014; 81(1):63-6.
CTFG. Recommendations related to contraception and pregnancy testing in clinical trials. 2014 [19 May 2020]; Available from: http://www.hma.eu/fileadmin/dateien/Human_Medicines/01-About HMA/Working Groups/CTFG/2014 09 HMA CTFG Contraception.pdf.
Ferreira C, Ziegler S, Gahl W. Generalized Arterial Calcification of Infancy. In: Adam M P, Ardinger H H, Pagon R A, Wallace S E, Bean L J H, Stephens K, et al., editors. GeneReviews((R)). Seattle (WA)2014.
Guy W. The Clinical Global Impression Scale. In: Rush Jr A J, First M B, Blacker D, editors. Handbook of Psychiatric Measures, 2008. Washington, DC: American Psychiatric Publishing, Inc; 1976. p. 90-2.
IOF. Autosomal Recessive Hypophosphatemic Rickets Type 2 (ARHR2). 2019 [19 May 2020]; Available from: https://www.iofbonehealth.org/osteoporosis-musculoskeletal-disorders/skeletal-rare-disorders/autosomal-recessive-hypophosphatemi-0.
Mackenzie N C, Huesa C, Rutsch F, MacRae VE. New insights into NPP1 function: lessons from clinical and animal studies. Bone. 2012 November; 51(5):961-8.
McDonald C M, Henricson E K, Abresch R T, Florence J M, Eagle M, Gappmaier E, et al. The 6-minute walk test and other endpoints in Duchenne muscular dystrophy: longitudinal natural history observations over 48 weeks from a multicenter study. Muscle Nerve. 2013 September; 48(3):343-56.
Montes J, McDermott M P, Mirek E, Mazzone E S, Main M, Glanzman A M, et al. Ambulatory function in spinal muscular atrophy: Age-related patterns of progression. PLoS One. 2018; 13(6):e0199657.
Moran J J. Idiopathic arterial calcification of infancy: a clinicopathologic study. Pathol Annu. 1975; 10:393-417.
Mylius C F, Paap D, Takken T. Reference value for the 6-minute walk test in children and adolescents: a systematic review. Expert Rev Respir Med. 2016 December; 10(12):1335-52.
NCI. National Cancer Institute Division of Cancer Treatment and Diagnosis (DCTD), National Cancer Institute (NCI), National Institutes of Health (NIH), Department of Health and Human Services (DHHS). Common Terminology Criteria for Adverse Events V5.0 (CTCAE). Published: Nov. 27, 2017. Available at https://ctep.cancer.gov/protocoldevelopment/electronic_applications/docs/ctcae_v5_quick_reference 8.5×11.pdf. 2017.
Orriss I R, Arnett T R, Russell R G. Pyrophosphate: a key inhibitor of mineralisation. Curr Opin Pharmacol. 2016 June; 28:57-68.
Rutsch F, Boyer P, Nitschke Y, Ruf N, Lorenz-Depierieux B, Wittkampf T, et al. Hypophosphatemia, hyperphosphaturia, and bisphosphonate treatment are associated with survival beyond infancy in generalized arterial calcification of infancy. Circ Cardiovasc Genet. 2008 December; 1(2):133-40.

### ENUMERATED EMBODIMENTS

The following exemplary embodiments are provided, the numbering of which is not to be construed as designating levels of importance:

Embodiment 1: A method of treating, ameliorating, preventing further development and/or progression of, and/or preventing diffuse idiopathic skeletal hyperostosis (DISH), Ankylosing Spondylitis, and/or Spondylarthritis in a patient in need thereof, the method comprising administering to the patient a therapeutically effective amount of a compound of formula (I), or a salt or solvate thereof:

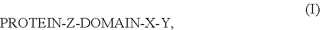

wherein in (I):
PROTEIN comprises the catalytic region of ENPP1;
DOMAIN is absent or at least one selected from the group consisting of a human IgG Fc domain (Fc), human serum albumin protein (ALB), and a fragment thereof;
X and Z are independently absent or a polypeptide comprising 1-20 amino acids, and
Y is a negatively charged bone-targeting sequence;
thereby treating, ameliorating, preventing further development and/or progression of, and/or preventing DISH, Ankylosing Spondylitis, and/or Spondylarthritis in the patient.

Embodiment 2: The method of Embodiment 1, wherein the patient has ENPP1 haploinsufficiency.

Embodiment 3: The method of Embodiment 1, wherein the patient does not have ENPP1 haploinsufficiency.

Embodiment 4: The method of any one of Embodiments 1-3, wherein the patient is not ENPP1 deficient.

Embodiment 5: The method of any one of Embodiments 1-3, wherein the patient is ENPP1 deficient.

Embodiment 6: The method of any one of Embodiments 1-5, wherein the patient is administered the compound by at least one route selected from the group consisting of oral, aerosol, inhalational, rectal, vaginal, transdermal, subcutaneous, intranasal, buccal, sublingual, parenteral, intrathecal, intragastrical, ophthalmic, pulmonary, and topical.

Embodiment 7: The method of any one of Embodiments 1-6, wherein the compound is intravenously or subcutaneously administered to the patient.

Embodiment 8: The method of any one of Embodiments 1-7, wherein administering the compound to the patient increases, or prevents further decrease of, the patient's extracellular pyrophosphate concentrations.

Embodiment 9: The method of any one of Embodiments 1-8, wherein administering the compound to the patient decreases, or prevents further increase of, one or more of calcification of Achilles tendon, spinal calcification, hip joint calcification, and bilateral calcification in the patient.

Embodiment 10: The method of any one of Embodiments 1-9, wherein the DOMAIN comprises Albumin.

Embodiment 11: The method of any one of Embodiments 1-10, wherein the DOMAIN comprises an IgG Fc domain.

Embodiment 12: The method of any one of Embodiments 1-11, wherein the PROTEIN lacks the ENPP1 transmembrane domain.

Embodiment 13: The method of any one of Embodiments 1-12, wherein the compound is administered to the patient as a pharmaceutical composition further comprising at least one pharmaceutically acceptable carrier.

Embodiment 14: The method of any one of Embodiments 1-13, wherein the patient is a mammal.

Embodiment 15: The method of Embodiment 14, wherein the mammal is a human.

Embodiment 16: The method of any one of Embodiments 1-15, wherein the PROTEIN comprises amino acid residues 99 (PSCAKE) to 925 (QED) of SEQ ID NO: 1.

Embodiment 17: The method of any one of Embodiments 1-16, wherein the PROTEIN comprises amino acid residues 1 to 833 of SEQ ID NO: 3.

Embodiment 18: The method of any one of Embodiments 1-16, wherein the PROTEIN comprises the amino acid sequence depicted in SEQ ID NO: 2.

Embodiment 19: The method of any one of Embodiments 1-16, wherein the PROTEIN comprises the amino acid sequence depicted in SEQ ID NO: 3 or 4 or 5.

Embodiment 20: The method of any one of Embodiments 1-19, wherein the DOMAIN increases the circulating half-life of the compound relative to the circulating half-life of the compound lacking the DOMAIN.

Embodiment 21: The method of any one of Embodiments 1-20, wherein the patient has also been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

Embodiment 22: The method of any one of Embodiments 1-20, wherein the patient has not been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

Embodiment 23: The method of any one of Embodiments 1-22, wherein the patient has a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

Embodiment 22: The method of any one of Embodiments 1-22, wherein the patient does not have a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

### INCORPORATION BY REFERENCE

All publications and patents mentioned herein are hereby incorporated by reference in their entirety as if each individual publication or patent was specifically and individually indicated to be incorporated by reference.

### Other Embodiments

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method of treating, ameliorating, or preventing further development or progression of diffuse idiopathic skeletal hyperostosis (DISH) or Spondylarthritis in a patient in need thereof, the method comprising administering to the patient a therapeutically effective amount of a compound of formula (I), or a salt or solvate thereof: 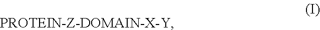
wherein in (I):
PROTEIN comprises the catalytic region of ENPP1;
DOMAIN is absent or at least one selected from the group consisting of a human IgG Fc domain (Fc), human serum albumin protein (ALB), and a fragment thereof;
X and Z are independently absent or a polypeptide comprising 1-20 amino acids, and
Y is a negatively charged bone-targeting sequence;
thereby treating, ameliorating, or preventing further development and/or progression of DISH and/or Spondylarthritis in the patient.

2. The method of claim 1, wherein the patient has ENPP1 haploinsufficiency.

3. The method of claim 1, wherein the patient does not have ENPP1 haploinsufficiency.

4. The method of claim 1, wherein the patient is not ENPP1 deficient.

5. The method of claim 1, wherein the patient is ENPP1 deficient.

6. The method of claim 1, wherein the patient is administered the compound by at least one route selected from the group consisting of oral, aerosol, inhalational, rectal, vaginal, transdermal, subcutaneous, intranasal, buccal, sublingual, parenteral, intrathecal, intragastrical, ophthalmic, pulmonary, and topical.

7. The method of claim 1, wherein the compound is intravenously or subcutaneously administered to the patient.

8. The method of claim 1, wherein administering the compound to the patient increases, or prevents further decrease of, the patient's extracellular pyrophosphate concentrations.

9. The method of claim 1, wherein administering the compound to the patient decreases, or prevents further increase of, one or more of calcification of Achilles tendon, spinal calcification, hip joint calcification, and bilateral calcification in the patient.

10. The method of claim 1, wherein the DOMAIN comprises Albumin.

11. The method of claim 1, wherein the DOMAIN comprises an IgG Fc domain.

12. The method of claim 1, wherein the PROTEIN lacks the ENPP1 transmembrane domain.

13. The method of claim 1, wherein the compound is administered to the patient as a pharmaceutical composition further comprising at least one pharmaceutically acceptable carrier.

14. The method of claim 1, wherein the patient is a mammal.

15. The method of claim 14, wherein the mammal is a human.

16. The method of claim 1, wherein the PROTEIN comprises amino acid residues 99 (PSCAKE) to 925 (QED) of SEQ ID NO: 1.

17. The method of claim 1, wherein the PROTEIN comprises amino acid residues 1 to 833 of SEQ ID NO: 3.

18. The method of claim 1, wherein the PROTEIN comprises the amino acid sequence depicted in SEQ ID NO: 2.

19. The method of claim 1, wherein the PROTEIN comprises the amino acid sequence depicted in SEQ ID NO: 3 or 4 or 5.

20. The method of claim 1, wherein the DOMAIN increases the circulating half-life of the compound relative to the circulating half-life of the compound lacking the DOMAIN.

21. The method of claim 1, wherein the patient has also been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.

22. The method of claim 1, wherein the patient has not been diagnosed with a disease or condition selected from the group consisting of Early onset osteoporosis, Osteopenia, Age related osteopenia, OPLL, Hereditary Hypophosphatemic Rickets, X-linked hypophosphatemia, Autosomal Recessive Hypophosphatemia Rickets type 2, Autosomal Dominant Hypophosphatemic Rickets, and Hypophosphatemic rickets.
